(19) United States
(12) Patent Application Publication
(10) Pub. No.: US 2009/0176815 A1
(43) Pub. Date: Jul. 9, 2009

(54) Novel Tricyclic Spiropiperidine Compounds, Their Synthesis and Their Uses as Modulators of Chemokine Receptor Activity

(76) Inventors: Tomas Eriksson, Lund (SE); Martin Hemmerling, Lund (SE); Bo-Goran Josefsson, Lund (SE); Svetlana Ivanova, Lund (SE); Marguerite Mensonides-Harsema, Lund (SE); John Mo, Lund (SE); John Pavey, Loughborough (GB); Austen Pimm, Loughborough (GB); James Reuberson, Slough (GB); Hakan Schulz, Lund (SE); Per Strandberg, Lund (SE)

Correspondence Address: FISH & RICHARDSON P.C., P.O BOX 1022, MINNEAPOLIS, MN, 55440-1022, US

(21) Appl. No.: 12/355,478

(22) Filed: Jan. 16, 2009

Related U.S. Application Data
(63) Continuation of application No. PCT/SE2007/000694, filed on Jul. 17, 2007.
(60) Provisional application No. 60/831,776, filed on Jul. 19, 2006.

(51) Int. Cl. A61K 31/438; C07D 491/107; A61P 11/00

(52) U.S. Cl. 514/278; 546/17

(57) ABSTRACT

Compounds of formula (I) wherein R¹, R³, R⁴, R⁵, R⁶, R⁷, and R¹⁰ are as defined in the specification, are described. The present invention also relates to pharmaceutical composition comprising said compounds and to the use of said compounds in therapy. The present invention further relates to processes for the preparation of said compounds and to new intermediates useful in the preparation thereof. Beside, the invention relates to salts and polymorphic forms of the new compounds as well as the preparation thereof.

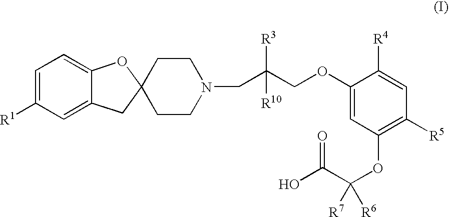

The present invention relates to new compounds, to pharmaceutical composition containing said compounds and to the use of said compounds in therapy. The present invention also relates to processes for the preparation of said compounds and to new intermediates useful in the preparation thereof. Furthermore, the invention relates to salts and polymorphic forms of the new compounds as well as the preparation thereof.

The essential function of the lungs requires a fragile structure with enormous exposure to the environment, including pollutants, microbes, allergens, and carcinogens. Host factors, resulting from interactions of lifestyle choices and genetic composition, influence the response to this exposure. Damage or infection to the lungs can give rise to a wide range of diseases of the respiratory system (or respiratory diseases). A number of these diseases are of great public health importance. Respiratory diseases include Acute Lung Injury, Acute Respiratory Distress Syndrome (ARDS), occupational lung disease, lung cancer, tuberculosis, fibrosis, pneumoconiosis, pneumonia, emphysema, Chronic Obstructive Pulmonary Disease (COPD) and asthma.

Among the most common respiratory diseases is asthma. Asthma is generally defined as an inflammatory disorder of the airways with clinical symptoms arising from intermittent airflow obstruction. It is characterised clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

COPD is a term which refers to a large group of lung diseases which can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells. The two most important conditions covered by COPD are chronic bronchitis and emphysema.

Chronic bronchitis is a long-standing inflammation of the bronchi which causes increased production of mucous and other changes. The patients' symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become is enlarged. These enlarged areas trap stale air and do not effectively exchange it with fresh air. This results in difficult breathing and may result in insufficient oxygen being delivered to the blood. The predominant symptom in patients with emphysema is shortness of breath.

WO01/098273 describes compounds having activity as pharmaceuticals, in particular as modulators of chemokine receptor (especially CCR1 chemokine receptor), salts thereof and pharmaceutical formulations, and their potential use in treating various diseases. A desirable property for a drug acting at the CCR1 receptor is that it has high potency e.g. as determined by its ability to inhibit the activity of the CCR1 receptor. It is also desirable for compounds to exhibit low activity against the human ether-a-go-go-related-gene (hERG)-encoded potassium channel. In this regard, low activity against hERG binding in vitro is indicative of low activity in vivo.

The present inventors have identified new compounds which modulate CCR1 receptor activity and which have particularly beneficial selectivity properties.

The CCR1 chemokine receptor CCR1 (chemokine receptor 1) is highly expressed in tissues affected in different autoimmune, inflammatory, proliferative, hyperproliferative and immunologically mediated diseases e.g. asthma and chronic obstructive pulmonary disease. Moreover, inflammatory cells (e.g. neutrophils and monocytes/macrophages) contribute to the pathogenesis of respiratory diseases such as COPD by secretion of proteolytic enzymes, oxidants and pharmacologic mediators. These cells are dependent on the function of CCR1 for recruitment and activation in lung tissues.

A range of tricyclic spiro piperidines or spiropyrrolidines which modulate the activation of chemokine receptors are described for example in WO2004/005295, WO2005/037814, WO2005/049620, WO2005/061499 and WO2005/054249.

A desirable property for a drug acting at the CCR1 receptor is that it has high potency e.g. as determined by its ability to inhibit the activity of the CCR1 receptor. It is also desirable for such drugs to possess good selectivity and pharmacokinetic properties in order to further enhance drug efficacy. As an example, it can be advantageous for such drugs to exhibit low activity against the human ether-a-go-go-related gene (hERG)-encoded potassium channel. In this regard, low activity against hERG binding in vitro is indicative of low activity in vivo.

### DESCRIPTION OF THE DRAWINGS

FIG. 1. The X-ray powder diffractogram S-enantiomer of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form A.

FIG. 2. The X-ray powder diffractogram R-enantiomer of 2-{2-Chloro-5-{[(2R)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid.

FIG. 3. The X-ray powder diffractogram S-enantiomer of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form B.

FIG. 4. The X-ray powder diffractogram S-enantiomer of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form C.

FIG. 5. The X-ray powder diffractogram S-enantiomer of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form D.

FIG. 6. The X-ray powder diffractogram S-enantiomer of 2-{2-Chloro-5-{[(2S)-3-(5-chloro-1′H, 3H-spiro[1-benzofuran-2,4′-piperidin]-1′-yl)-2-hydroxypropyl]oxy}-4-[(methylamino)carbonyl]phenoxy}-2-methylpropanoic acid Form F.

FIG. 7. The X-ray powder diffractogram S-enantiomer of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form G.

FIG. 8. The X-ray powder diffractogram S-enantiomer of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid hydrochloride.

FIG. 9. The X-ray powder diffractogram S-enantiomer of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid sodium hydroxide.

In accordance with the present invention, there is provided a compound of formula

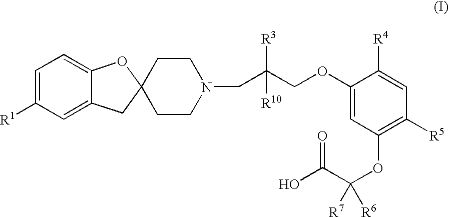

wherein:
R¹ is halogen;
R³ is hydrogen or hydroxyl;
R¹⁰ is hydrogen or C₁₋₃alkyl;
R⁴ is —CONR⁸R⁹, —N(H)C(O)R¹¹ or —N(H)C(O)NR⁸R⁹, where R⁸ and R⁹ are independently selected from hydrogen, C₁₋₆ alkyl or C₃₋₇cycloalkyl, or R⁸ and R⁹ together with the nitrogen atom to which they are attached, form a 4-7 membered heterocyclic ring which is optionally substituted with one or more hydroxy groups;
R¹¹ is C₁₋₆alkyl, C₂₋₆alkenyl, C₃₋₆cycloalkyl, adamantyl, C₅₋₆cycloalkenyl, phenyl or a saturated or unsaturated 5-10 membered heterocyclic ring system comprising at least one heteroatom selected from nitrogen, oxygen, and sulphur, each of which may be optionally substituted by one or more substituents independently selected from nitro, hydroxyl, oxo, halo, carboxyl, C₁₋₆ alkyl, C₁₋₆alkoxy, C₁₋₆alkylthio, C₁₋₆alkycarbonyl, C₁₋₆alkoxycarbonyl, phenyl or —NHC(O)R²;
R² is C₁₋₆alkyl, amino or phenyl;
R⁵ is hydrogen or halo;
R⁶ and R⁷ are independently selected from hydrogen or C₁₋₆alkyl, or R⁶ and R⁷ together with the carbon atom to which they are attached form a 3-7 membered saturated cycloalkyl group, or a pharmaceutically acceptable salt thereof.

Compounds of the present invention show good CCR1 and CCR3 inhibitory activity. In addition they have particularly low affinity for the human ether-a-go-go-related gene (hERG)-encoded potassium channel and therefore are advantageous with regard to safety windows.

In one embodiment R¹ is chlorine or fluorine. In another embodiment R¹ is chlorine.

In yet another embodiment R³ is hydroxyl.

In a further embodiment R¹⁰ is hydrogen or methyl. For instance R¹⁰ is hydrogen.

In one embodiment R⁴ is —CONR⁸R⁹ or —N(H)C(O)NR⁸R⁹, where R⁸ and R⁹ are as defined above. Suitable groups R⁸ and R⁹ are selected from hydrogen or C₁₋₆alkyl, such as methyl. In one embodiment, R⁸ is hydrogen and R⁹ is methyl. In another embodiment R⁸ and R⁹ are both methyl.

In another embodiment R⁸ and R⁹ together with the nitrogen atom to which they are attached, form a 4-7 membered heterocyclic ring which is optionally substituted with one or more hydroxy groups. Examples of heterocyclic groups for R⁸ and R⁹ and the nitrogen atom to which they are attached include azetininyl, pyrrolidinyl or piperadinyl, and pyrrolidinyl.

In an alternative embodiment R⁴ is a group —N(H)C(O)R¹¹ where R¹¹ is as defined above. In one embodiment R¹¹ is selected from hydrogen, C₁₋₆alkyl or C₃₋₇cycloalkyl. For example, R¹¹ is hydrogen or C₁₋₆ alkyl such as methyl. In another embodiment, R¹¹ is C₁₋₆alkyl such as methyl.

In yet a further embodiment R⁵ is hydrogen and chlorine. For instance, R⁵ is chlorine. In another embodiment R⁵ is hydrogen.

In one embodiment R⁶ and R⁷ are independently selected from hydrogen or C₁₋₆alkyl, such as methyl. For instance, R⁶ and R⁷ are both methyl, or R⁶ and R⁷ are both hydrogen.

In another embodiment R⁶ and R⁷ together with the carbon atom to which they are attached form a 3-7 membered saturated cycloalkyl group, such as cyclopropyl or cyclohexyl.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, neo-pentyl, n-hexyl or i-hexyl. The term C₁₋₄ alkyl having 1 to 4 carbon atoms and may be but are not limited to methyl, ethyl, n-propyl, i-propyl or tert-butyl.

In this specification, unless stated otherwise, the term “alkenyl” includes both straight and branched chain alkenyl groups. The term “C₂₋₆alkenyl” having 2 to 6 carbon atoms and one or two double bonds, may be, but is not limited to vinyl, allyl, propenyl, butenyl, crotyl, pentenyl, or hexenyl, and a butenyl group may for example be buten-2-yl, buten-3-yl or buten-4-yl.

The term “alkoxy”, unless stated otherwise, refers to radicals of the general formula —O—R, wherein R is selected from a hydrocarbon radical. The term “alkoxy” may include, but is not limited to methoxy, ethoxy, propoxy, isopropoxy, butoxy, t-butoxy, isobutoxy, cyclopropylmethoxy, allyloxy or propargyloxy.

In this specification, unless stated otherwise, the term “cycloalkyl” refers to an optionally substituted, partially or completely saturated monocyclic, bicyclic or bridged hydrocarbon ring system. The term “C₁₋₆cycloalkyl” may be, but is not limited to cyclopropyl, is cyclobutyl, cyclopentyl or cyclohexyl.

In this specification, unless stated otherwise, the term “cycloalkenyl” refers to an optionally substituted, partially unsaturated monocyclic, bicyclic or bridged hydrocarbon ring system. The term “C₅₋₆cycloalkenyl” may be, but is not limited to cyclopentenyl or cyclohexenyl.

In this specification, unless stated otherwise, the term “3-7 membered saturated cycloalkyl group”, refers to a ringsystem having, in addition to carbon atoms, zero to three heteroatoms, including the oxidized form of nitrogen and sulfur and any quaternized form of a basic nitrogen, including, but not limited to cyclopropane, oxirane, cyclobutane, azetidine, cyclopentane, cyclohexane, benzyl, furane, thiophene, pyrrolidine, morpholine, piperidine, piperazine, pyrazine or azepane.

The term “a saturated or unsaturated 5-10 membered heterocyclic ring system comprising at least one ring heteroatom selected from nitrogen, oxygen, and sulphur” and the term “a 4-7 membered heterocyclic ring” refer to a hydrocarbon moiety comprising one to three fused rings, optionally having 6, 10 or 14 π atoms shared in a cyclic array and having, in addition to carbon atoms, zero to five heteroatoms. Fused ringsystems may include, but are not limited to, 8-azabicyclo[3.2.1]octane, 3-azabicyclo[3.2.1]octane, 2-azabicyclo[2.2.2]octane, indole, indoline, benzofuran, benzothiophene, naphtalene, chroman, quinazoline, phenoxazine, azulene, adamantane, anthracene or phenoxazine.

In this specification, unless stated otherwise, the term “amine” or “amino” refers to radicals of the general formula —NRR′, wherein R and R′ are independently selected from hydrogen or a hydrocarbon radical.

In this specification, unless stated otherwise, the terms “halo” and “halogen” may be fluorine, iodine, chlorine or bromine.

It will be appreciated that throughout the specification, the number and nature of substituents on rings in the compounds of the invention will be selected so as to avoid sterically undesirable combinations.

Compounds of the present invention have been named with the aid of computer software (ACDLabs 8.0/Name(IUPAC)).

Particular examples of compounds of formula (I) are compounds of formula (IA)

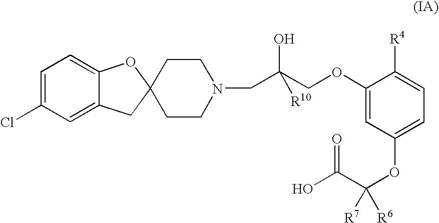

where R⁴, R⁶, R⁷ and R¹⁰ are as defined above, or a pharmaceutically acceptable salt thereof.

Other examples of compounds of formula (I) are compounds of formula (IB)

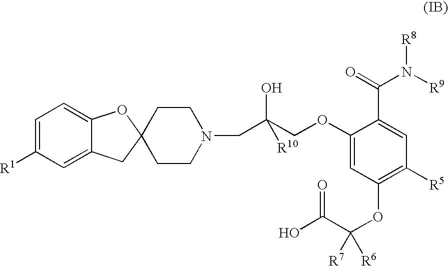

where R¹, R⁵, R⁶, R⁷, R⁸, R⁹ and R¹⁰ are as defined above, or a pharmaceutically acceptable salt thereof.

Compounds of formula (I) may include an asymmetric centre and be chiral in nature. Where the compound is chiral, it may be in the form of a single stereoisomer, such as a enantiomer, or it may be in the form of mixtures of these stereoisomers in any proportions, including racemic mixtures. Therefore, all enantiomers, diastereomers, racemates and mixtures thereof are included within the scope of the invention. The various optical isomers may be isolated by separation of a racemic mixture of the compounds using conventional techniques, for example, fractional crystallisation, or HPLC. Alternatively the optical isomers may be obtained by asymmetric synthesis, or by synthesis from optically active starting materials.

For example, stereoisomeric forms of the compound of formula (I) occur where R³ is hydroxy. Such compounds are suitably in the form of S isomers of formula (IC)

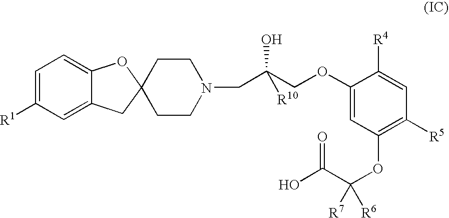

where R¹, R⁴, R⁵, R⁶, R⁷ and R¹⁰ are as defined above or a pharmaceutically acceptable salt thereof.

In another embodiment, the compounds of the invention are selected from
(4-(acetylamino)-2-chloro-5-{[(2S)-3-(5-chloro-1′H,3H-spiro[1-benzofuran-2,4′-piperidin]-1′-yl)-2-hydroxypropyl]oxy}phenoxy)acetic acid;
(4-(acetylamino)-3-{[(2S)-3-(5-chloro-1′H,3H-spiro[1-benzofuran-2,4′-piperidin]-1′-yl)-2-hydroxypropyl]oxy}phenoxy)acetic acid;
(4-(acetylamino)-2-chloro-5-{[(2S)-3-(5-fluoro-1′H,3H-spiro[1-benzofuran-2,4′-piperidin]-1′-yl)-2-hydroxypropyl]oxy}phenoxy)acetic acid;
{2-Chloro-5-{[(2S)-3-(5-chloro-1′H,3H-spiro[1-benzofuran-2,4′-piperidin]-1-yl)-2-hydroxypropyl]oxy}-4-[(methylamino)carbonyl]phenoxy}acetic acid;
2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid;
{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}acetic acid;
2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methylpropanoic acid;
(2-Chloro-5-{[(2S)-3-(5-chloro-1′H,3H-spiro[1-benzofuran-2,4′-piperidin]-1′-yl)-2-hydroxypropyl]oxy}-4-f{[(3S)-3-hydroxypyrrolidin-1-yl]carbonyl}phenoxy)acetic acid;
2-{2-Chloro-5-{[(2R)-3-(5-chloro-1′H,3H-spiro[1-benzofuran-2,4′-piperidin]-1-yl)-2-hydroxypropyl]oxy}-4-[(methylamino)carbonyl]phenoxy}-2-methylpropanoic acid;
2-{2-Chloro-5-{3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoic acid; and
2-[5-{[(2S)-3-(7-tert-Butyl-5-chloro-1′H,3H-spiro[1-benzofuran-2,4′-piperidin]-1′-yl)-2-hydroxypropyl]oxy}-2-chloro-4-(methylcarbamoyl)phenoxy]-2-methylpropanoic acid,
   or a pharmaceutically acceptable salt thereof.

It will be appreciated also that the compounds of formula (I) and salts thereof may exist as zwitterions (internal salts). Accordingly, the representation of formula (I) and the examples of the present invention also covers these zwitterionic forms.

The compounds of formula (I) and pharmaceutically acceptable salts thereof may exist in solvated, for example hydrated, as well as unsolvated forms, and the present invention encompasses all such forms.

Compounds of formula (I) above may be converted to a pharmaceutically acceptable salt thereof, preferably an acid addition salt such as a hydrochloride, hydrobromide, phosphate, sulfphate, acetate, ascorbate, benzoate, fumarate, hemifumarate, furoate, succinate, maleate, tartrate, citrate, oxalate, xinafoate, methanesulphonate, p-toluenesulphonate, benzenesulphonate, ethanesulphonate, 2-naphthalenesulfonate, mesytilenesulfonate, nitric acid, 1,5-naphthalene-disulphonate, p-xylenesulphonate, aspartate or glutamate.

They may also include basic addition salts such as an alkali metal salt for example sodium or potassium salts, an alkaline earth metal salt for example calcium or magnesium salts, a transition metal salt such as a zinc salt, an organic amine salt for example a salt of triethylamine, diethylamine, morpholine, N-methylpiperidine, N-ethylpiperidine, piperazine, procaine, dibenzylamine, N,N-dibenzylethylamine, choline or 2-aminoethanol or amino acids for example lysine or arginine.

A pharmaceutically acceptable salt also includes internal salt (zwitterionic) forms. One embodiment relates to a compound of the invention compound which is in zwitterionic forms.

In another embodiment, the compounds of the invention are selected from
(4-(acetylamino)-2-chloro-5-{[(2S)-3-(5-fluoro-1′H,3H-spiro[1-benzofuran-2,4′-piperidin]-1′-yl)-2-hydroxypropyl]oxy}phenoxy)acetic acid, hydrochloride;
2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid sodium hydroxide;
2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methylpropanoic acid hydrochloride;
2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoic acid trifluoracetate;
2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoic acid p-toluensulfonate;
2-{2-Chloro-5-{3-(5-chloro-1′H,3H-spiro[1-benzofuran-2,4′-piperidin-1′-yl)-2-hydroxypropyl]oxy}-4-[(dimethylamino)carbonyl]phenoxy}-2-methyl-propanoic acid trifluoracetate; and
2-[5-{[(2S)-3-(7-tert-Butyl-5-chloro-1′H,3H-spiro[1-benzofuran-2,4′-piperidin]-1′-yl)-2-hydroxypropyl]oxy}-2-chloro-4-(methylcarbamoyl)phenoxy]-2-methylpropanoic acid trifluoroacetate.

One embodiment of the invention relates to 2-{2-Chloro-5-f{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid.

In the formulation of drug compositions, it is important for the drug substance to be in a form in which it can be conveniently handled and processed. This is of importance, not only from the point of view of obtaining a commercially-viable manufacturing process, but also from the point of view of subsequent manufacture of pharmaceutical formulations comprising the active compound.

Further, in the manufacture of drug compositions, it is important that a reliable, reproducible and constant plasma concentration profile of drug is provided following administration to a patient.

Furthermore, some crystalline forms may be more suitable for certain ways of administration e.g. inhalation, than others. Also the dosing profile of some crystalline forms may differ from others.

Chemical stability, solid state stability, and “shelf life” of the active ingredients are also very important factors. The drug substance, and compositions containing it, should preferably be capable of being effectively stored over appreciable periods of time, without exhibiting a significant change in the active component's physico-chemical characteristics (e.g. its chemical composition, density, hygroscopicity and solubility). Moreover, it is also important to be able to provide drug in a form, which is as chemically pure as possible.

The skilled person will appreciate that, typically, if a drug can be readily obtained in a stable form, such as a stable crystalline form, advantages may be provided, in terms of ease of handling, ease of preparation of suitable pharmaceutical compositions, and a more reliable solubility profile.

In an embodiment of the invention, the compound of formula (I) or salt thereof is in a substantially pure crystalline form e.g. at least 40% crystalline, at least 50% crystalline, at least 60% crystalline, at least 70% crystalline or at least 80% crystalline. Crystallinity can be estimated by conventional X-ray diffractometry techniques.

In another embodiment of the invention, the compound of formula (I) or salt thereof is from 40%, 50%, 60%, 70%, 80% or 90% to 95%, 96%, 97%, 98%, 99% or 100% crystalline.

It should be noted that where X-ray powder diffraction peaks are expressed herein (in is degrees 2θ), the margin of error is consistent with the United States Pharmacopeia general chapter on X-ray diffraction (USP941)—see the United States Pharmacopeia Convention. X-Ray Diffraction, General Test <941>. United States Pharmacopeia, 25th ed. Rockville, Md.: United States Pharmacopeial Convention; 2002:2088-2089).

One embodiment of the invention relates to the compound 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid,

which exhibits at least the following characteristic X-ray powder diffraction peaks (expressed in degrees 2θ) (Form A):
(1) 5.1, 10.2 and 12.9, or
(2) 5.1, 8.9 and 13.2, or
(3) 8.9, 10.2, 12.9, 15.1, 17.0 and 21.2 or
(4) 5.1, 8.9, 10.2, 14.6, 15.4, 21.2 and 25.8 or
(5) 5.1, 8.9, 10.2, 12.6, 14.6, 15.1 and 17.0 or
(6) 5.1, 10.2, 12.6, 13.2, 14.6, 15.1, 17.0, 17.9, 21.2 and 21.8 or
(7) 5.1, 8.9, 10.2, 12.6, 13.2, 14.6, 14.9, 16.4, 19.2, 21.8 and 27.1 or
(8) 5.1, 8.9, 10.2, 12.6, 12.9, 13.2, 14.6, 14.9, 15.1, 15.4, 16.4, 17.9, 19.2, 20.0, 21.8 and 25.8.

Another embodiment of the invention relates to the compound 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid,

which exhibits at least the following characteristic X-ray powder diffraction peaks (expressed in degrees 2θ) (Form C):
(1) 4.5, 8.9 and 12.8, or
(2) 4.5, 8.6 and 10.6, or
(3) 4.5, 8.9, 10.6, 12.8, 14.8 and 17.6 or
(4) 8.6, 8.9, 12.8, 13.9, 15.7, 16.6 and 18.8 or
(5) 4.5, 8.6, 8.9, 10.6, 13.9, 15.7, 16.0, 16.6 and 17.9 or
(6) 4.5, 8.9, 10.6, 12.8, 13.9, 14.8, 15.7, 17.6, 18.8 and 20.0 or
(7) 4.5, 8.6, 8.9, 10.6, 12.8, 13.9, 15.7, 16.0, 16.6, 17.9, 18.8, 20.0, 20.9 and 21.2.

A further embodiment of the invention relates to the compound 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid,

which exhibits at least the following characteristic X-ray powder diffraction peaks (expressed in degrees 2θ) (Form F):
(1) 7.5, 9.2 and 10.7, or
(2) 7.5, 8.9 and 11.1, or
(3) 7.5, 8.9, 9.2, 11.1, 12.2 and 16.3 or
(4) 8.9, 9.2, 10.7, 11.1, 11.7, 12.2 and 15.1 or
(5) 7.5, 8.9, 9.2, 10.7, 11.7, 12.2, 13.8, 15.1, 16.7 and 18.5 or
(6) 7.5, 8.9, 9.2, 11.1, 11.9, 13.8, 15.1, 16.3, 17.8, 18.3, 18.7 and 20.9 or
(7) 7.5, 8.9, 9.2, 10.7, 11.1, 11.7, 12.2, 13.8, 15.1, 18.3, 18.7, 19.7, 21.4, 22.3 and 24.0 or
(8) 7.5, 9.2, 10.7, 11.7, 11.9, 12.2, 13.8, 15.1, 16.3, 16.7, 17.8, 18.3, 19.2, 19.7, 20.9, 21.4 and 22.3.

One embodiment of the invention relates to the compound 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid,

which exhibits at least the following characteristic X-ray powder diffraction peaks (expressed in degrees 2θ) (Form G):
(1) 4.8, 12.2 and 15.4, or
(2) 4.8, 9.7 and 13.7, or
(3) 9.7, 13.7, 14.5, 15.6, 17.1 and 20.3 or
(4) 4.8, 13.7, 14.5, 15.4, 16.3, 17.1 and 20.3 or
(5) 4.8, 9.7, 13.7, 14.5, 15.6, 16.3 and 19.7 or
(6) 9.7, 12.2, 13.7, 14.5, 15.6, 16.3, 19.4, 20.3, 21.4 and 23.1 or
(7) 9.7, 13.7, 14.5, 15.6, 16.3, 19.7, 20.3, 20.8, 21.4, 23.1 and 25.5 or
(8) 4.8, 9.7, 12.2, 13.7, 15.4, 16.3, 17.1, 19.4, 19.7, 20.3, 20.8, 21.4, 23.1 and 25.5.

Another embodiment of the invention relates to the compound 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid hydrochloride,

which exhibits at least the following characteristic X-ray powder diffraction peaks (expressed in degrees 2θ):
(1) 7.6, 7.9, 20.6, 21.3, 22.9 and 23.8 or
(2) 9.7, 13.7, 14.5, 16.2, 16.4, 19.6, 20.6, 21.3, 22.4, 22.9 and 23.8 or
(3) 5.5, 7.6, 7.9, 13.4, 14.5, 15.2, 15.9, 16.2, 16.4, 19.6, 20.6, 21.3, 22.4, 22.9 and 23.8.

A further embodiment of the invention relates to the compound 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid sodium hydroxide,

which exhibits at least the following characteristic X-ray powder diffraction peaks (expressed in degrees 2θ):
(1) 7.6, 8.6 and 18.4 or
(2) 5.6, 7.6, 8.6, 13.1, 17.0 and 18.4.

Another embodiment relates to the substantially pure compound 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid having an X-ray powder diffraction pattern substantially the same as that shown in FIG. 1 to 9.

### Process

Compounds of the present invention can be prepared by routes, which are analogous to those described in WO2004/005295.

The present invention further provides a process for the preparation of a compound of formula (I) or a pharmaceutically acceptable salt thereof, as defined above which comprises;

(a) where R³ is a hydroxyl group, reacting a compound of formula (II)

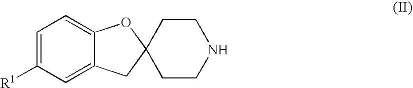

where R¹ is as defined in compounds of formula (I), with a compound of formula (III)

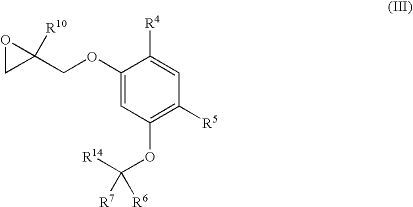

where R⁴, R⁵, R⁶, R⁷ and R¹⁰ are as defined in compounds of formula (I), or a protected derivative thereof, and R¹⁴ is carboxy or a protected derivative thereof; or
(b) where R³ is a hydroxyl group, reacting a compound of formula (IV)

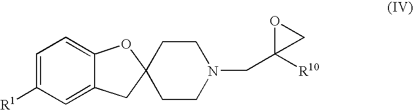

where R¹ and R¹⁰ are as defined in compounds of formula (I), with a compound of formula (V)

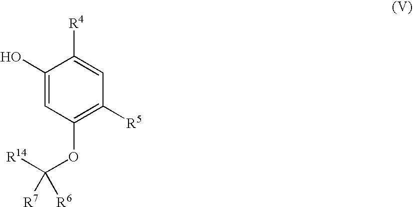

where R⁴, R⁵, R⁶ and R⁷ are as defined in compounds of formula (I), in the presence of a suitable base, and R¹⁴ is carboxy or a protected derivative thereof: or
(c) reacting a compound of formula (II) as defined above, with a compound of formula (VI)

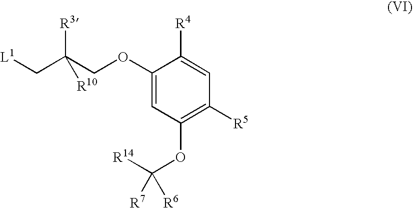

wherein L¹ is a leaving group (such as a hydroxyl group, p-toluenesulphonyloxy (tosylate) or methylsulphonyloxy (mesylate)) R⁴, R⁵, R⁶, R⁷ and R¹⁰ are as defined in compounds of formula (I), and R¹⁴ is carboxy or a protected derivative thereof, R^{3′} is R³ as defined in compounds of formula (I) or —O—P where P is a suitable protecting group,
(d) reacting a compound of formula (VII)

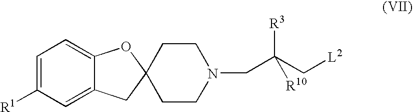

where R¹, R³ and R¹⁰ are as defined in compounds of formula (I), L² is a suitable leaving group, such as halogen, in particular chlorine, with a compound of formula (V) as defined above; in the presence of a suitable base,
(e) when R⁴ represents a group —N(H)C(O)R¹¹, reacting a compound of formula (IX)

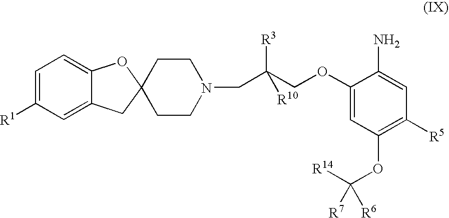

where R¹, R³, R⁶, R⁷ and R¹⁰ are as defined in compounds of formula (I) and R¹⁴ is carboxy or a protected derivative thereof,
with a compound of formula (X)

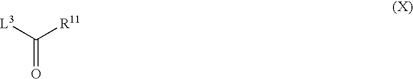

where R¹¹ is as defined in compounds of formula (I), and L³ is a leaving group (such as hydroxyl or halogen, for example chlorine);
(f) when R⁴ represents a group —CONR⁸R⁹, reacting a compound of formula (XI)

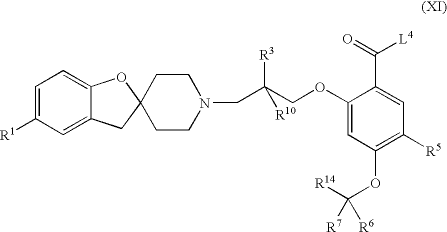

where R¹, R³, R⁵, R⁶, R⁷ and R¹⁰ are as defined in compounds of formula (I), R¹⁴ is carboxy or a protected derivative thereof and L⁴ is a leaving group (such as hydroxy or halogen, for example chlorine) with a compound of formula (XII)

HNR⁸R⁹ (XII)

where R⁸ and R⁹ are as defined in compounds of formula (I);
(g) reacting a compound of formula (XIII)

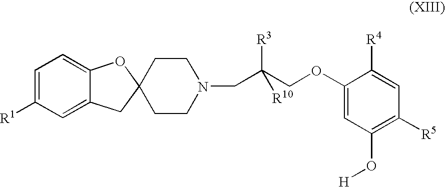

where R¹, R³, R⁴, R⁵ and R¹⁰ are as defined in compounds of formula (I), with a compound of formula (XIV)

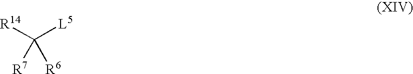

where R⁶ and R⁷ are as defined in compounds of formula (I), L⁵ is a leaving group such as halogen, in particular bromine, and R¹⁴ is carboxy or a protected derivative thereof in the presence of a suitable base;
and thereafter, if desired or necessary, carrying out one or more of the following steps
(i) converting a compound of formula (I) obtained to a different compound of formula (I);
(ii) removing any protecting groups; and
(iii) forming a pharmaceutically acceptable salt of the compound of formula (I).

As will be apparent to a person skilled in the art, when an S-enantiomer (i.e. compounds with the S configuration at the stereocentre with R³ as hydroxy attached) is synthesised, the intermediates III, IV, VI, VII, IX, XI and XIII can have the relevant S configuration to ensure the configuration is maintained in the final product.

Processes (a) to (f) may conveniently be carried out in a solvent, e.g. an organic solvent such as an alcohol (e.g. methanol or ethanol), a hydrocarbon (e.g. toluene), THF or cyanides (e.g. acetonitrile or butyronitrile) at a temperature of, for example, 15° C. or above such as a temperature in the range from 20 to 120° C.

Process (b) typically requires the use of a base such as sodium hydride. Other suitable bases may be used, for example lithium diisopropylamine or lithium hexamethyldisilazide.

In process (d), the choice of a suitable leaving group L² would be routine for a person skilled in the art. A suitable leaving group may, for example, be formed by the reaction of the compound of formula (XV)

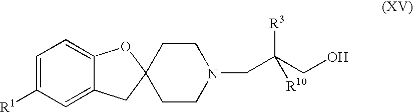

where R¹, R³ and R¹⁰ are as defined in compounds of formula (I), with DEAD (diethyl azodicarboxylate) and Ph₃P. However, the use of other leaving groups (e.g. Cl, Br, tosylate (4-toluenesulfonate), mesylate (methanesulfonate) are possible.

Process (d) or process (g) may typically require the use of a base such as potassium carbonate or cesium carbonate, or any other appropriate base such as tertiary amines N-ethyldiisopropylamine or 1,4-diazabicyclo[2.2.2]octane (DABCO).

It will be appreciated by those skilled in the art that in the processes of the present invention certain functional groups such as carboxyl, hydroxyl, or amino groups in the starting reagents or intermediate compounds may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3rd edition, T. W. Greene and P. G. M. Wuts, Wiley-Interscience (1999).

For example, in the processes of the invention, R¹⁴ may be COOP′, where P′ is a suitable protecting group (e.g. methyl or ethyl). After the reaction, the ester can be hydrolysed to afford the required acid functionality (or salt thereof). However, a person skilled in the art would recognise that a carboxy group may be protected by other functional groups (other than esters) which upon their removal, affords the required acid functionality (or salt thereof).

Compounds of formulae (II), (IV), (VII), (X), (XII), (XIII) and (XIV) are either commercially available, or known in the literature (in particular from WO2004/005295) or may be prepared easily using known techniques.

Intermediates of formula (III), (V), (VI), (IX) and (XI) and salts thereof are novel and comprise an independent aspect of the invention. They can generally be prepared using conventional methods.

One embodiment relates to a compound of formula (III)

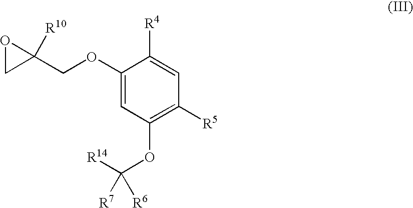

where R⁴, R⁵, R⁶, R⁷ and R¹⁰ are as defined in compounds of formula (I), or a protected derivative thereof, and R¹⁴ is carboxy or a protected derivative thereof, or a salt thereof.

Another embodiment relates to a compound of formula (V)

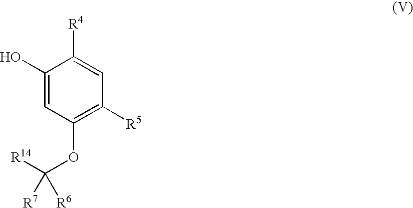

where R⁴, R⁵, R⁶ and R⁷ are as defined in compounds of formula (I), in the presence of a suitable base, and R¹⁴ is carboxy or a protected derivative thereof.

A further embodiment relates to a compound of formula (VI)

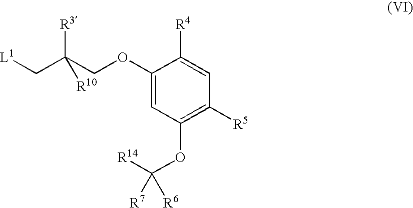

wherein L¹ is a leaving group, R⁴, R⁵, R⁶, R⁷ and R¹⁰ are as defined in compounds of formula (I), R¹⁴ is carboxy or a protected derivative thereof, R^{3′} is R³ are as defined in compounds of formula (I) or —O—P where P is a protecting group, or a salt thereof.

Yet another embodiment relates to a compound of formula (IX)

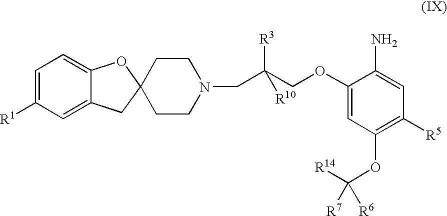

where R¹, R³, R⁵, R⁶, R⁷ and R¹⁰ are as defined in compounds of formula (I) and R¹⁴ is carboxy or a protected derivative thereof, or a salt thereof.

Yet a further embodiment relates to a compound of formula (XI)

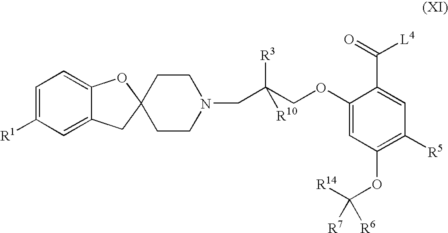

where R¹, R³, R⁵, R⁶, R⁷ and R¹⁰ are as defined in compounds of formula (I), R¹⁴ is carboxy or a protected derivative thereof and L⁴ is a leaving group, or a salt thereof.

In particular however, compounds of formula (III) are suitably prepared using routes, which involve nucleophilic aromatic substitution reactions (SnAr).

For example, compounds of formula (III) may be prepared from compounds of formula (XVI)

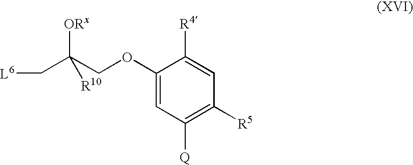

where R⁵ and R¹⁰ are as defined in compounds of formula (I), R^{4′} is R⁴ as defined in compounds of formula (I) or a nitro group or amino group, R^{x} is hydroxy or a hydroxy protecting group and Q is OH, OP (where P is an alcohol-protecting group) or OC(R⁶)(R⁷)(R¹⁴) where R⁶, R⁷ and R¹⁴ are as defined in compounds of formula (I). Suitable examples of leaving groups L⁶ include sulfonate, tosylate, nosylate and mesylate as well as halo such as bromide. Suitable hydroxy protecting groups R^{x} include acetyl.

The activated diols of formula (XVI) can be transformed to the epoxides upon treatment with a base using standard techniques. Suitable alkali metal bases include, but are not limited to, potassium carbonate, sodium hydroxide, potassium hydroxide, sodium hydride, sodium methoxide and sodium ethoxide.

Where Q is a group OH or OP it may be subsequently converted to a group OC(R⁶)(R⁷)(R¹⁴) where R⁶, R⁷ and R¹⁴ are as defined in compounds of formula (I), by reaction with a compound of formula (XIV) in the presence of a base as described above in relation to process (g). Where R⁴ is nitro, it may be reduced to amino and subsequently acetylated to form a group R⁴ using conventional chemical methods. Similarly, where R^{4′} is amino, it may be converted to a group R⁴ by acetylation using conventional chemical methods.

Compounds of formula (XVI) are suitably prepared by activation of a compound of formula (XVII)

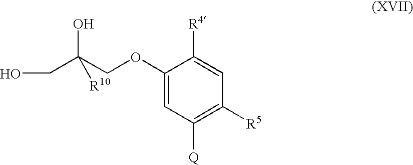

where R^{4′} and Q are as defined in compounds of formula (XVI) and R⁵ and R¹⁰ are as defined in compounds of formula (I), for example by reaction with a compound of formula R^{x}L⁶ such as HBr or acetyl bromide in acetic acid, and base (typically alkali metal bases, including, but not limited to, potassium carbonate, sodium hydroxide, potassium hydroxide, sodium hydride, sodium methoxide and sodium ethoxide).

Compounds of formula (XVII) may be prepared by deprotection of a compound of formula (XVIII)

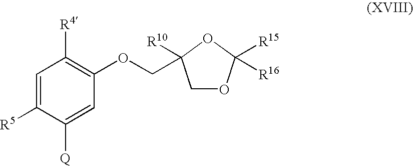

where R^{4′} and Q are as defined in compounds of formula (XVI), R⁵ and R¹⁰ are as defined in compounds of formula (I), and R¹⁵ and R¹⁶ together with the carbon atom to which both are attached form a 1,2 diol protecting group.

The 1,2 diol protecting group formed by R¹⁵ and R¹⁶ can be chosen such that its removal can provide the corresponding 1,2 diol. 1,2 diol-protecting groups and methods for their removal are well known in the art. For example, methods to effect deprotection of 1,2 diol-protecting groups are outlined in ‘Protective Groups in Organic Synthesis’, 3rd edition, T. W. Greene and P. G. M. Wutz, Wiley-Interscience (1999). For example removal of the diol protecting group may be effected using acid catalysed hydrolysis using acids such as HCl, acetic acid, para-toluene sulfonic acid or ion exchange resins such as Dowex 50, to give the 1,2 diol of formula (XVII).

R¹⁵ and R¹⁶ may, for example, each independently represent hydrogen or C₁₋₆alkyl (e.g. methyl or ethyl), or R¹⁵ and R¹⁶, together with the carbon atom to which they are both attached may form a C₄₋₇cycloalkyl ring, more preferably a cyclopentyl or cyclohexyl ring. Alternatively, R¹⁵ may be hydrogen or methyl with R¹⁶ being phenyl. Alternatively still, R¹⁵ may be hydrogen or methyl with R¹⁶ being 4-methoxyphenyl.

In one embodiment, R¹⁵ and R¹⁶ are both methyl.

Compounds of formula (XVIII) where R⁴ is nitro, amino, N(H)C(O)R¹¹ or N(H)C(O)NR⁸R⁹ where R⁸, R⁹ and R¹¹ are as defined in compounds of formula (I) are suitably prepared by reacting a compound of formula (XIX)

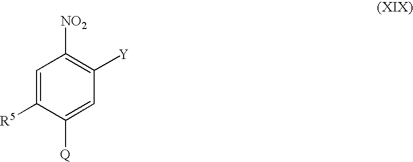

where R⁵ is as defined in compounds of formula (I), Q is as defined in compounds of formula (XVI) and Y is chlorine or fluorine, with a compound of formula (XX)

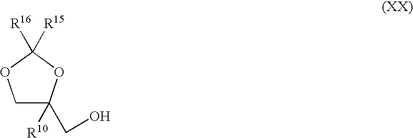

where R¹⁰ is as defined in compounds of formula (I), R¹⁵ and R¹⁶ are as defined in compounds of formula XVIII. Thereafter the nitro group may be reduced to amino and acetylated to a group N(H)C(O)R¹¹ or N(H)C(O)NR⁸R⁹ as required using conventional chemical methods.

Alternatively, compounds of formula (XVIII) may be prepared by reacting a compound of formula (XXI)

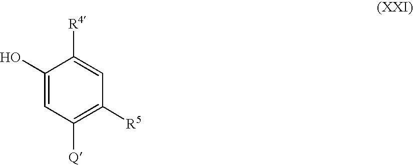

where R⁵ is as defined in compounds of formula (I), R⁴ is as defined in compounds of formula (XVI) and Q′ is OP where P is an alcohol-protecting group or OC(R⁶)(R⁷)(R¹⁴) and where R⁴, R⁵, R⁶, R⁷ and R¹⁴ are as defined in compounds of formula (I), with a compound of formula (XXII)

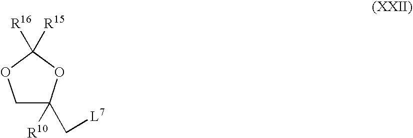

wherein R¹⁰ are as defined in compounds of formula (I), R¹⁵, R¹⁶ together with the carbon atom to which both are attached form a 1,2 diol protecting group and L⁷ is a suitable leaving group. Examples of suitable groups L⁷ include p-toluenesulphonyloxy (tosylate) or methylsulphonyloxy (mesylate). The reaction is suitably carried out in a suitable solvent (such as, but not limited to, DMF or acetonitrile) in the presence of a suitable base (such as, but not limited to, cesium carbonate or a tertiary amine like N-ethyldiisopropylamine) at a temperature of, for example, 15° C. or above such as a temperature in the range from 20 to 120° C.

Compounds of formula (XXI) or (V) where R⁴ is a group CONR⁸R⁹ and R⁵ is halo such as chloro are suitably prepared by reacting a compound of formula (XXV),

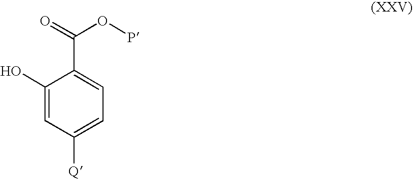

in which P′ is a suitable carboxylic acid protecting group, like, but not limited to, a methyl or ethyl ester, and Q′ is OP where P is an alcohol-protecting group or OC(R⁶)(R⁷)(R¹⁴) and where R⁶, R⁷ and R¹⁴ are as defined in compounds of formula (I), with a halogenating agent, and thereafter, transformation of the group COOP′ to a group C(O)NR⁸R⁹ where R⁸ and R⁹ are as defined in compounds of formula (I).

Suitable halogenating agents include chlorinating agents, like, but not limited to, Cl₂ or SO₂Cl₂ either neat or in a suitable solvent like DCM or DMF. The halogenation reaction is suitably effected at a temperature of, for example, 15° C. or above such as a temperature in the range from 0 to 120° C. This may be followed by transformation of COOP′ to R⁴, where R⁴ is as defined in compounds of formula (I), using standard techniques like, but not limited to, reacting to amine in a suitable solvent at a temperature of, for example, 15° C. or above such as a temperature in the range from 20 to 120° C.

Compounds of formulae (V), (VI), (IX) and (XI) may be prepared by reacting a compound of formula (XIV) as defined above with compounds (XVII), (XVIII), (XXIX) and (XXX) respectively.

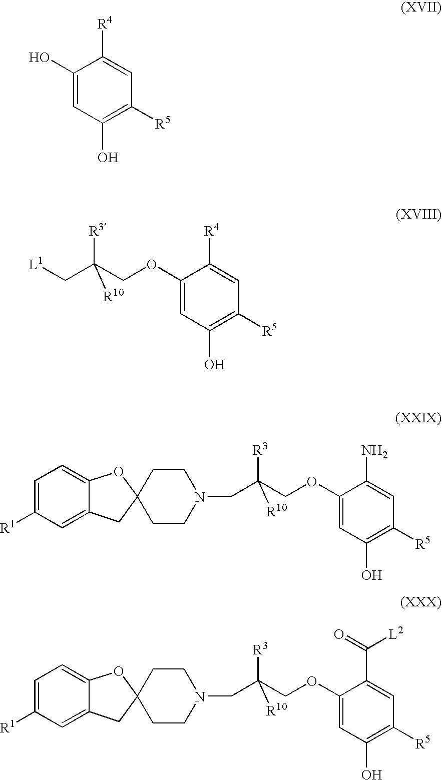

where R¹, R³, R⁴, R⁵, R¹⁰, L¹ and L² are as defined above.

Compounds (XVII), (XVIII), (SIX) and (XX) are also known for example from WO2004/005295 or they can be prepared using methods analogous to those described in that application, the content of which is incorporated herein by reference.

### Alternative Process 1

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid may also be prepared using an alternative process as shown below in schemes 1 to 3.

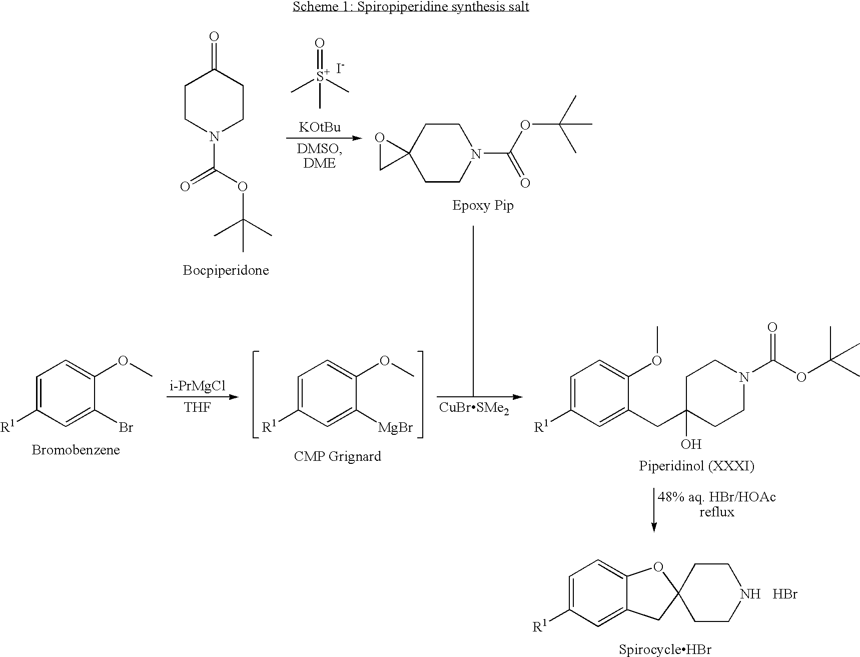

One embodiment of the invention relates to the preparation of spiropiperidine comprising the following steps;

h) reacting bocpiperidone with trimethylsulfoxonium iodide to form an epoxy piperidine in the presence of a base,
i) reacting 2-Bromo-4-chloroanisole with isopropylmagnesium chloride to form the aryl Grignard reagent, which is then reacted with the epoxy piperidine to form a piperidinol in the presence of a catalyst, and
j) reacting piperidinol with hydrobromic acid to obtain spiropiperidine.

A person skilled in the art would recognise that solvents, bases, Grignard reagents and catalysts may be used in the process according to scheme 1.

Suitable bases that may be used for the preparation of the Epoxy pip are, but not limited to, LiOR^{x}, NaOR^{x}, KOR^{x}, where R^{x} is C₁₋₆ alkyl such as for example tert-butoxide. Suitable solvents that may be used for the preparation of the Epoxy pip are, but not limited to, dimethylsulphoxide, THF, diethyl ether, tert-butyl methyl ether, dimethoxyethane, dimethylacetamide, NMP or toluene.

Suitable Grignard reagents that may be used in the process for making the aryl Grignard reagent in Scheme 1 include but are not limited to, compounds of formula R^{y}MgR^{v} or R^{v}₂Mg, wherein R^{y} represents Cl, Br or I and R^{v} represents C₁₋₆ alkyl, C₃₋₇ cycloalkyl or optionally substituted phenyl such as for example isopropylamagnesium chloride. Suitable catalysts that may be used in the process for making the piperidinol include but are not limited to copper (I) chloride, copper (I) bromide, copper (I) bromide dimethyl sulphide complex, copper (I) iodide or copper (I) cyanide.

Suitable solvents that may be used in the process for making the piperidinol include but are not limited to THF, 2-methyltetrahydrofuran, diethyl ether, tert-butyl methyl ether, dimethoxyethane, toluene or hexanes.

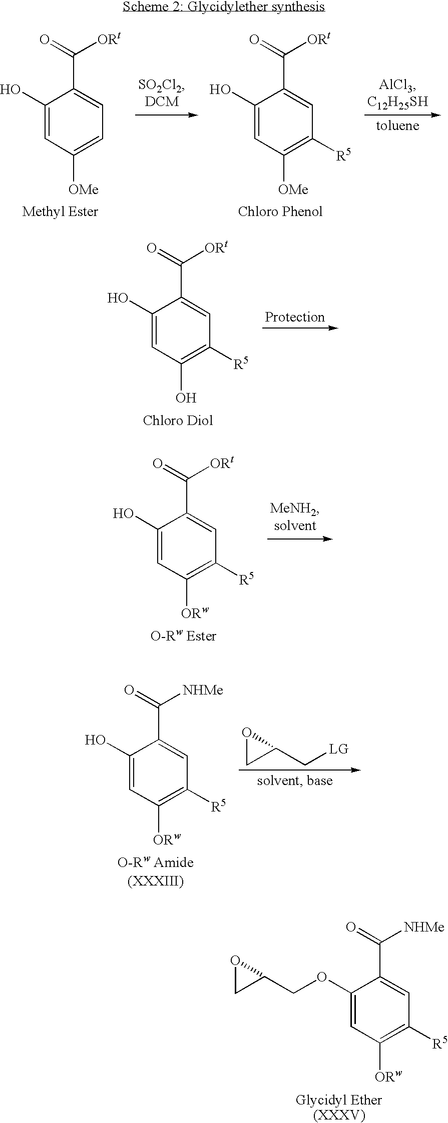

where R^{t} may be any substituent providing an ester function such as for example C₁₋₆ alkyl such as methyl, ethyl, etc., R^{w} is any suitable protection group such as for example PMB and R⁵ is as defined in compounds of formula (I).

A person skilled in the art would recognise that solvent, bases and catalyst may be used in the process according to scheme 2.

Suitable protecting groups and conditions for their application are described in ‘Protective Groups in Organic Chemistry’, edited by J. W. F. McOmie, Plenum Press (1973) and ‘Protective Groups in Organic Synthesis’, 3rd edition, T. W. Greene and P. G. M. Wuts, Wiley-Interscience (1999).

Suitable bases that may be used in the process for making the O-w ester where R^{w} is PMB include but are not limited to cesium carbonate, potassium carbonate, 1,8-Diazabicyclo[5.4.0]undec-7-ene, triethylamine, ethyldiisopropylamine or sodium hydride. Suitable solvents that may be used in the process for making the O—R^{w} ester where R^{w} is PMB include but are not limited to dichloromethane, toluene, N,N-dimethylformamide, N-methylpyrrolidone, tert-butyl methyl ether, methanol, ethanol, isopropanol and acetonitrile. Suitable solvents that may be used in the process for making the compound of formula XXXIII include but are not limited to THF, water, methanol, ethanol, isopropanol, or mixtures thereof such as a water/THF mixture.

Suitable bases that may be used for the preparation of the compound of formula XXXV are, but not limited to cesium carbonate, potassium carbonate, sodium hydride or potassium tert-butoxide.

Suitable solvents that may be used in the process for making the compound of formula XXXV include but are not limited to butyronitrile, acetonitrile, toluene, tetrahydrofuran, DMF or NMP.

Another embodiment of the invention relates to the preparation of the compound of formula XXXV comprising the following steps;

k) reacting O—R^{w} ester with methylamine to obtain the compound of formula XXXIII,

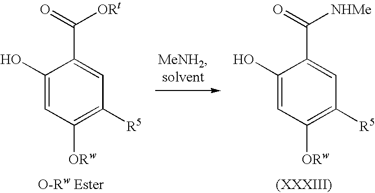

where R⁵, R^{t} and R^{w} are defined as above,
1) reacting the compound of formula XXXIII with an epoxide to form the compound of formula XXXV,

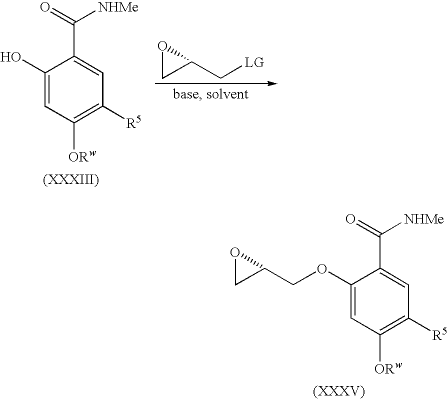

where R⁵ and R^{w} is defined as above and LG is halogen, SO₂R^{u} where R^{u}=C₁₋₆alkyl such as methyl, ethyl, etc., or optionally substituted aryl such as phenyl, tosyl or 3-nitrophenyl. Suitable epoxides may be glycidyl nosylate, optically pure epichlorohydrin, glycidyl tosylate, glycidyl benzenesulphonate or glycidyl mesylate.

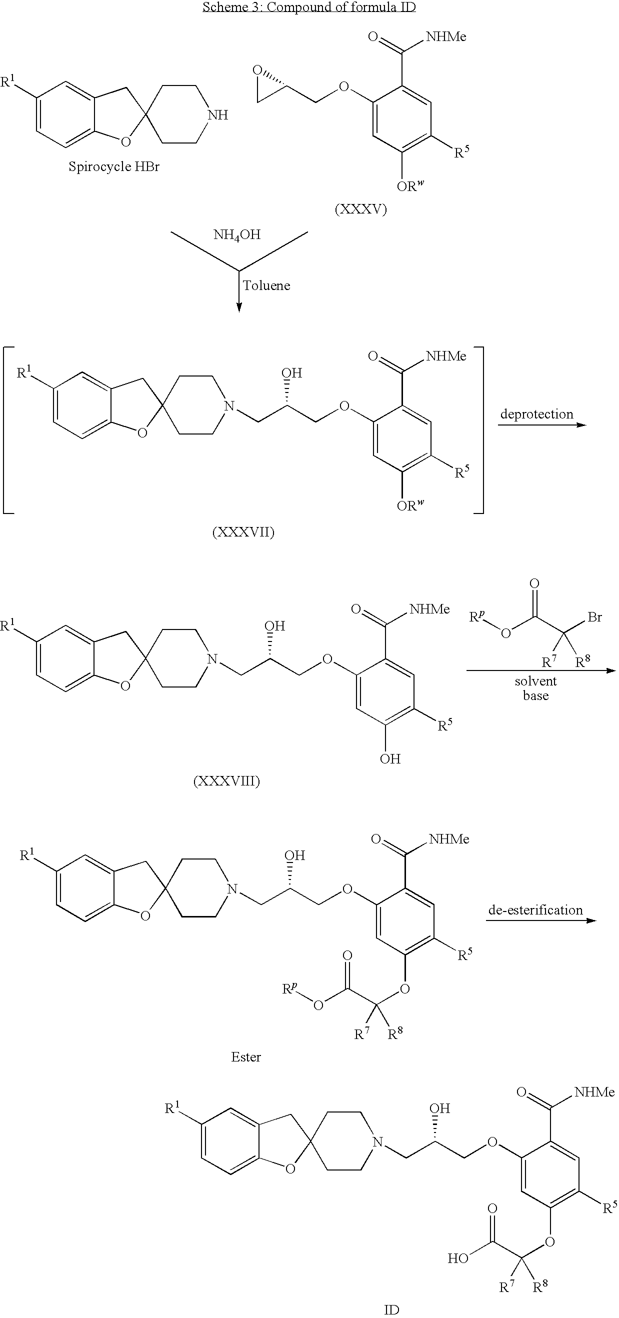

where R¹ to R⁸, R^{t}, R^{w} are defined as above and R^{p} may be hydrogen or any substituent providing an ester function such as for example C₁₋₆ alkyl such as methyl, ethyl, etc.

Another embodiment of the invention relates to the preparation of the compounds of formula ID comprising the following steps;

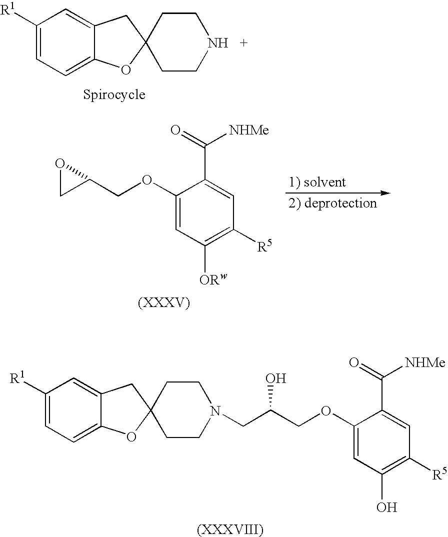

m) treatment of a solution of the spiropiperidine HBr salt with aqueous ammonium is hydroxide to liberate the free base and then reacting this with the compound of formula XXXV in a suitable solvent followed by deprotection to obtain the compound of formula XXXVIII, optionally as a salt,
n) reacting the compound of formula XXXVIII with α-bromo carboxylic ester in a suitable solvent in the presence of a base at an elevated temperature, preferably at a temperature of 55-70° C., and subsequently de-esterification with a solution of a base followed by isolation by filtration after pH adjustment.

In the case where R^{p} is hydrogen, the compound of formula XXXVIII would be reacted with an α-bromocarboxylic acid in a suitable solvent in the presence of a base at an elevated temperature. De-esterification would not be needed and the compound of formula ID would be isolated after pH adjustment.

A person skilled in the art would recognise that solvent, bases and reagents may be used in the process according to scheme 3.

Suitable bases that may be used in the process for making the compound of formula XXXVII include but are not limited to ammonium hydroxide, sodium hydroxide, potassium hydroxide, sodium carbonate or potassium carbonate.

Suitable solvents that may be used in the process for making the compound of formula XXXVII include but are not limited to ethyl acetate, isopropyl acetate, toluene, THF, ethanol, methanol or isopropanol.

The deprotection of protecting groups is described in ‘Protective Groups in Organic Chemistry’, edited by J. W. F. McOmie, Plenum Press (1973) and ‘Protective Groups in Organic Synthesis’, 3rd edition, T. W. Greene and P. G. M. Wuts, Wiley-Interscience (1999).

Suitable acids that may be used in the process for making the compound of formula XXXVIII where R^{w} is PMB include but are not limited to trifluoroacetic acid, formic acid, acetic acid or hydrochloric acid.

Suitable solvents that may be used in the process for making the compound of formula XXXVIII where R^{w} is PMB include but are not limited to DCM, toluene, tert-butyl methyl ether or THF.

Suitable bases that may be used in the process for making the ester include but are not limited to cesium carbonate, potassium carbonate or sodium hydride.

Suitable solvents that may be used in the process for making the ester include but are not limited to DMF, NMP, ethanol, methanol or isopropanol.

Suitable bases that may be used in the process for making the compound of formula ID include but are not limited to lithium hydroxide, sodium hydroxide or potassium hydroxide. Alternatively, some ester groups, for example where R^{p} is tert-butyl, can be de-esterified with acid and suitable acids which may be used for making the compound of formula ID in such cases are TFA, formic acid, acetic acid or hydrochloric acid. Suitable solvents that may be used in the process for making the compound of formula ID include but are not limited to water, methanol, ethanol, isopropanol or mixtures thereof such as for example a water/ethanol mixture.

Another embodiment of the invention relates to the preparation of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid comprising the following steps;

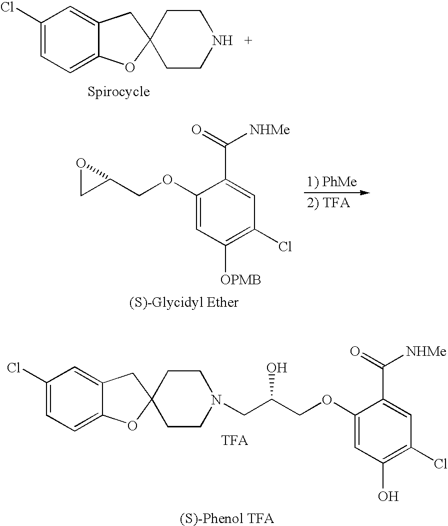

m-a) treatment of a solution of the spiropiperidine HBr salt with aqueous ammonium hydroxide to liberate the free base and then reacting this with the glycidylether in a suitable solvent followed by TFA treatment to obtain 5-chloro-2-{[(2S)-3-(5-chloro-1′H,3H-spiro[1-benzofuran-2,4′-piperidin]-1′-yl)-2-hydroxypropyl]oxy}-4-hydroxy-N-methylbenzamide, as its TFA salt,

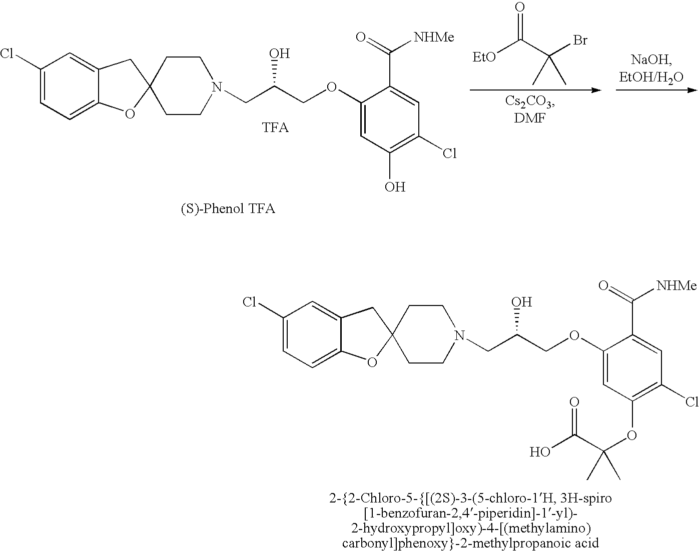

n-a) reacting 5-Chloro-2-{[(2S)-3-(5-chloro-1′H,3H-spiro[1-benzofuran-2,4′-piperidin]-1′-yl)-2-hydroxypropyl]oxy}-4-hydroxy-N-methylbenzamide TFA with ethyl-2-bromoisobutyrate in a suitable solvent in the presence of a base at an elevated temperature, preferably at a temperature of 55-70° C. Subsequently, the obtained product is redissolved in ethanol and treated with a solution of sodium hydroxide, whereafter the solvent is evaporated and the residue treated with aqueous ammonium acetate, filtered and washed with water/ethanol and then filtered.

Alternatively, after reaction with ethyl-2-bromoisobutyrate and de-esterification, aqueous citric acid is added and the solid filtered off, washed with water followed by ethanol, then recrystallised from ethanol/NMP and then from aqueous NMP. The first recrystallisation from an ethanol/NMP mixture reduces the level of the polymeric impurity to a low level. The second recrystallisation from a water/NMP mixture provides the product in form A.

Compounds of formulae (XXXI) to (XXXIX) and salts thereof are novel and comprise an independent aspect of the invention.

One embodiment relates to compound of formula XXXI, where R¹ is defined as in compounds of formula I

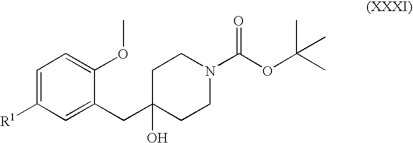

Another embodiment relates to compound 4-(5-Chloro-2-methoxybenzyl)-4-hydroxypiperidine-1-carboxylic acid, tert-butyl ester.

A further embodiment relates to compound of formula XXXII where R¹ is defined as in compounds of formula I

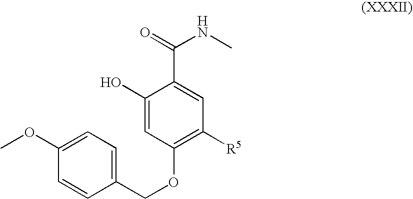

One embodiment relates to compound 5-Chloro-2-hydroxy-4-(4-methoxybenzyloxy)-N-methylbenzamide.

A further embodiment relates to a compound of formula XXXII, where R⁵ is defined as in compounds of formula I and R^{w} is hydrogen or any suitable protecting group, or a salt thereof.

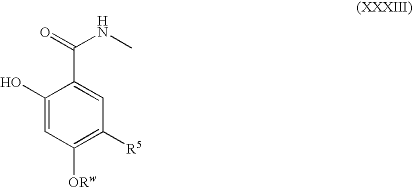

Another embodiment relates to compound of formula XXXIV, where R⁵ is defined as in compounds of formula I

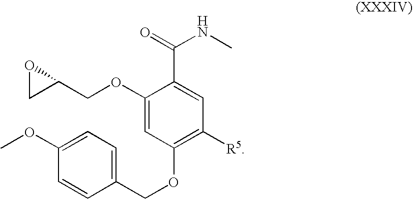

One embodiment relates to compound 5-Chloro-4-(4-methoxy-benzyloxy)-N-methyl-2-((S)-1-oxiranylmethoxy)benzamide.

Another embodiment relates to a compound of formula XXXV, where R^{w} is defined as in compounds of formula I and R^{w} is hydrogen or any suitable protecting group, or a salt thereof.

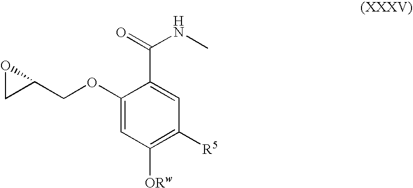

A further embodiment relates to a compound of formula XXXVI, where R¹ and R⁵ are defined as in compounds of formula I

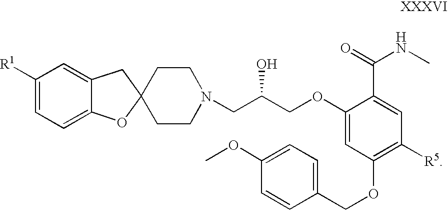

Yet a further embodiment relates to compound 5-Chloro-2-{[(2S)-3-(5-chloro-3H-spiro[1-benzofuran-2,4′-piperidin]-1′-yl)-2-hydroxypropyl]oxy}-4-(p-methoxybenzyloxy)-N-methylbenzamide.

Yet another embodiment relates to a compound of formula XXXVII, where R¹ and R⁵ are defined as in compounds of formula I and R^{w} is as defined hereinbefore

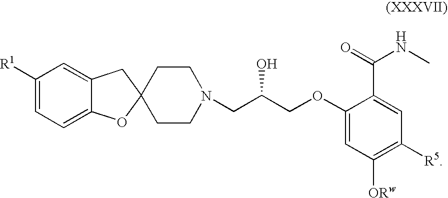

One embodiment relates to compound of formula XXXVIII, where R¹ and R⁵ are defined as in compounds of formula I

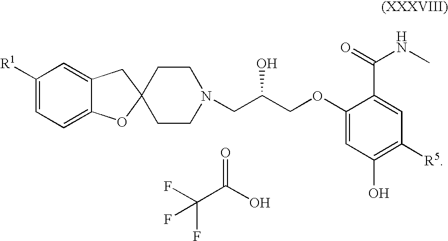

Another embodiment relates to 5-Chloro-2-{[(2S)-3-(5-chloro-3H-spiro[1-benzofuran-2,4′piperidin]-1′-yl)-2-hydroxypropyl]oxy}-4-hydroxy-N-methylbenzamide, trifluoroacetic acid salt.

Yet a further embodiment relates to compound of formula ID, where R¹ to R⁸ are defined as in compounds of formula I and R^{p} is as defined hereinbefore

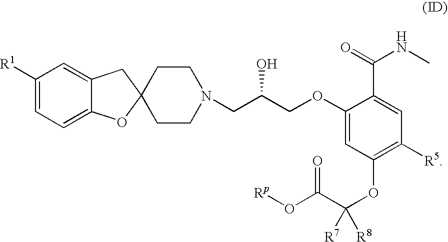

Examples of protecting groups are, but not limited to, alkyl (e.g. C₁₋₆ alkyl), ether (e.g. methoxymethyl, tetrahydropyranyl), optionally substituted arylalkyl (e.g. benzyl or para-methoxybenzyl) and silyl groups of formula (R^{q})₃Si— where each R^{q} independently represents an alkyl (e.g. C₁₋₆ alkyl) or aryl (e.g. phenyl) group, for example, tert-butyldimethylsilyl or triethylsilyl.

### Alternative Process 2

A further alternative process for preparation of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid is shown in scheme 4. Through an earlier alkylation of the phenolic hydroxy group the protection/deprotection steps can be avoided, making the overall process two steps shorter than the process given in Schemes 1-3.

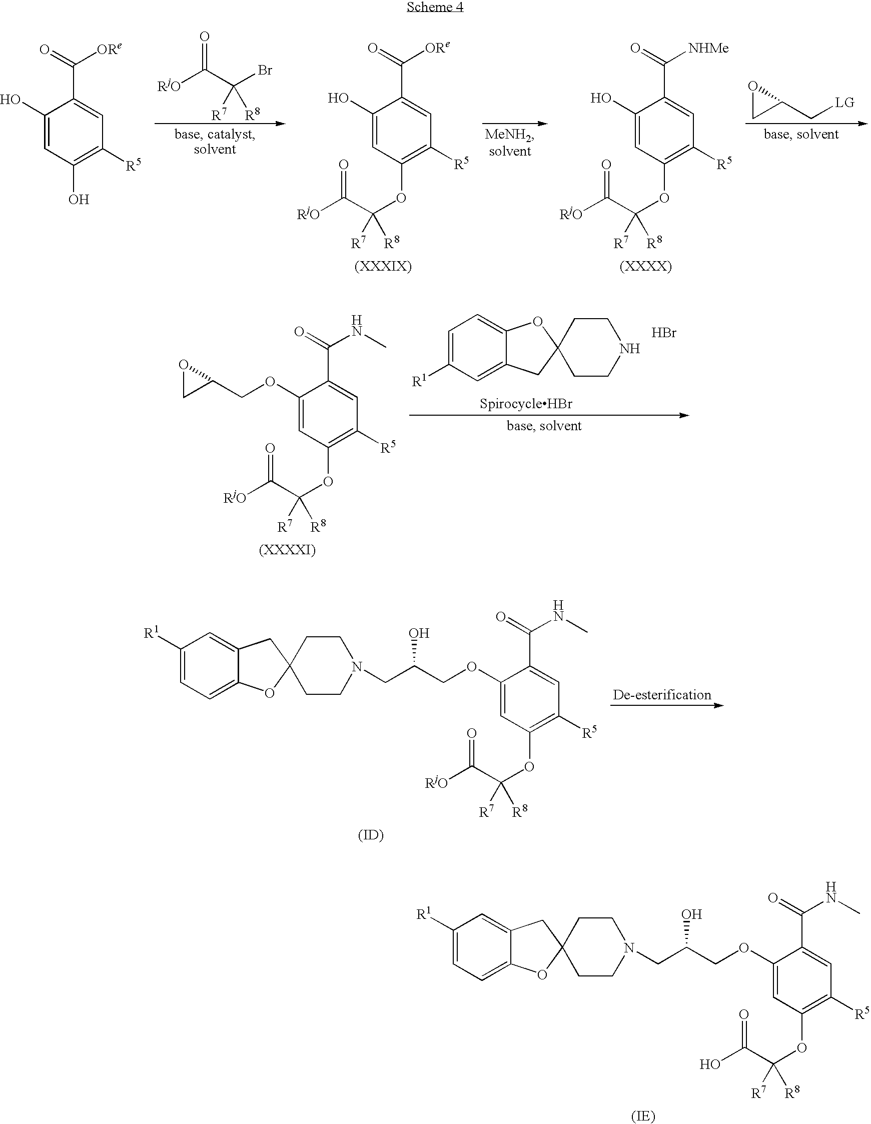

wherein R^{e} and R^{j} are independently any substituent forming an ester group such as, but not limited, to C₁₋₆ alkyl, optionally substituted arylalkyl e.g. benzyl or R^{j} is hydrogen.

Another embodiment of the invention relates to the preparation of the compound of formula IE comprising the following steps;

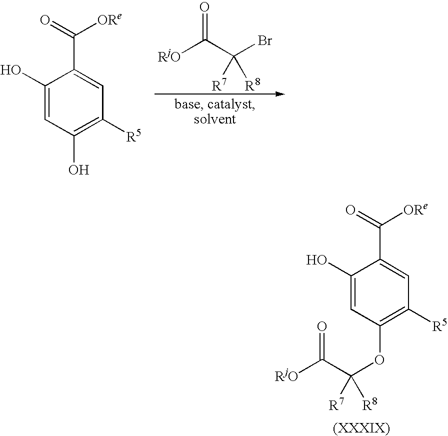

o) reacting the benzoic acid ester with an α-bromocarboxylic ester or α-bromocarboxylic acid in the presence of a base to form the compound of formula XXXIX,

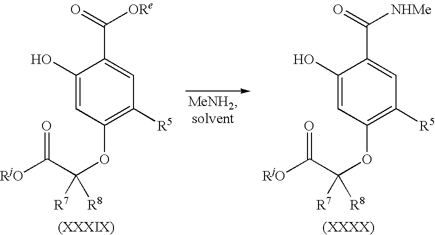

p) reacting the compound of formula XXXIX with a solution of methylamine to provide the compound of formula XXXX,

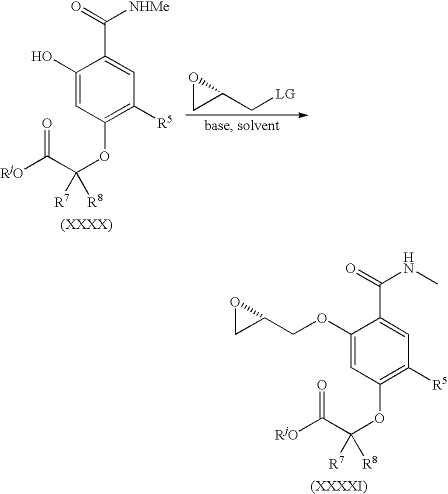

q) reacting the compound of formula XXXX with the epoxide to give the compound of formula XXXXI,

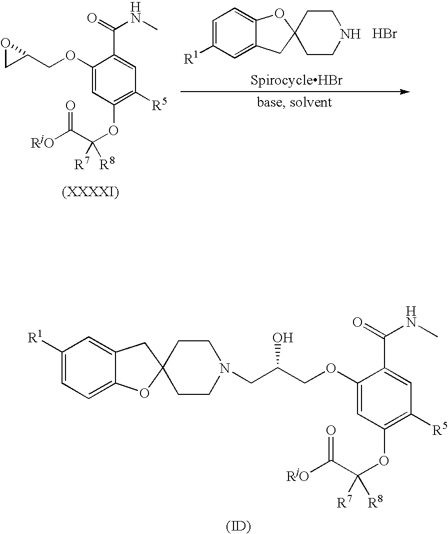

r) reacting the spirocycle with the compound of formula XXXXI to afford the compound of formula ID, and

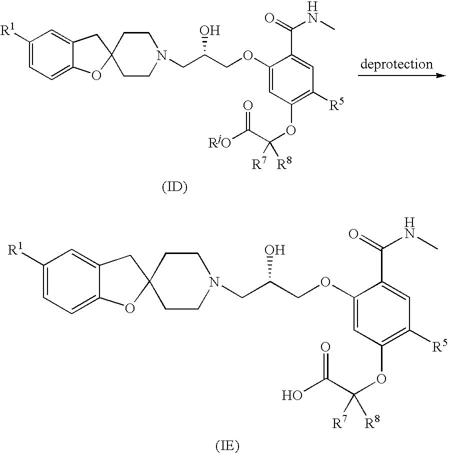

s) de-esterification of the compound of formula ID to provide the compound of formula IE in the cases where R^{p} is not hydrogen.

A person skilled in the art would recognise that solvents, bases and catalysts may be used in the process according to scheme 4.

Suitable solvents that may be used in the process for making the compound of formula XXXIX include but are not limited to DMF, NMP, ethanol, methanol or isopropanol.

Suitable bases that may be used in the process for making the compound of formula XXXIX include but are not limited to cesium carbonate, potassium carbonate or sodium hydride.

Suitable solvents that may be used in the process for making the compound of formula XXXX include but are not limited to dichloromethane, toluene, N,N-dimethylformamide, N-methylpyrrolidone, tert-butyl methyl ether, methanol, ethanol, isopropanol, acetonitrile, water or mixtures thereof.

Suitable solvents that may be used in the process for making the compound of formula XXXXI include but are not limited to butyronitrile, acetonitrile, toluene, tetrahydrofuran, DMF, NMP or mixtures thereof.

Suitable bases that may be used in the process for making the compound of formula XXXXI include but are not limited to caesium carbonate, potassium carbonate or sodium hydride.

Suitable solvents that may be used in the process for making the compound of formula ID include but are not limited to ethyl acetate, isopropyl acetate, toluene, THF, ethanol, methanol, isopropanol or mixtures thereof.

Suitable bases that may be used in the process for making the compound of formula ID include but are not limited to ammonium hydroxide, sodium hydroxide, potassium hydroxide, sodium carbonate or potassium carbonate.

Suitable solvents that may be used in the process for making the compound of formula IE include but are not limited to DCM, toluene, tert-butyl methyl ether, THF or mixtures thereof.

Depending on the nature of the group R^{j}, de-esterification under either acidic or basic conditions may be appropriate. Suitable acids that may be used in the process for making the compound of formula IE where R^{j} is tert-butyl include but are not limited to TFA, formic acid, acetic acid or hydrochloric acid.

Compounds of formulae (XXXIX) to (XXXXI) and ID and IE and salts thereof are novel and comprise an independent aspect of the invention.

One embodiment relates to compound of formula XXXIX or a salt thereof, where R¹ to R⁸ are defined as in formula I wherein R^{e} and R^{j} are independently any substituent forming an ester group such as, but not limited, to C₁₋₆ alkyl, optionally substituted arylalkyl e.g. benzyl or R^{j} is hydrogen.

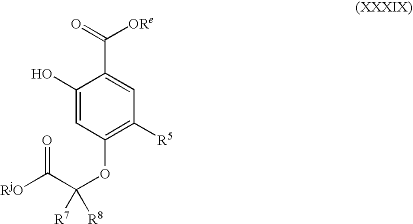

Another embodiment relates to compound 4-(1-tert-Butoxycarbonyl-1-methylethoxy)-5-chloro-2-hydroxybenzoic acid, methyl ester

A further embodiment relates to the compound of formula XXXX or a salt thereof, where R¹ to R⁸ are defined as in formula I and R^{j} is hydrogen or any substituent forming an ester group such as, but not limited, to C₁₋₆ alkyl, optionally substituted arylalkyl e.g. benzyl, etc.

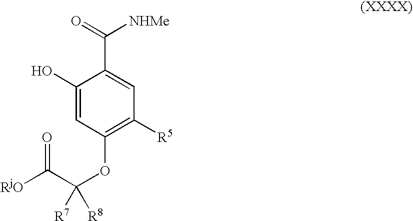

One embodiment relates to compound 2-(2-Chloro-5-hydroxy-4-methylcarbamoylphenoxy)-2-methylpropionic acid, tert-butyl ester.

Another embodiment relates to a compound of formula XXXXI, or a salt thereof, where R¹ to R⁸ are defined as in formula I and R¹ is hydrogen or any substituent forming an ester group such as, but not limited, to C₁₋₆ alkyl, optionally substituted arylalkyl e.g. benzyl, etc.

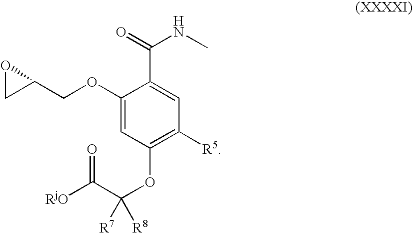

A further embodiment relates to compound 2-[2-Chloro-4-methylcarbamoyl-5-((S)-1-oxiranylmethoxy)-phenoxy]-2-methylpropionic acid, tert-butyl ester.

Another embodiment relates to a compound of formula ID, or a salt thereof, where R¹ to R⁸ are defined as in formula I and R^{j} is hydrogen or any substituent forming an ester group such as, but not limited, to C₁₋₆ alkyl, optionally substituted arylalkyl e.g. benzyl, etc.

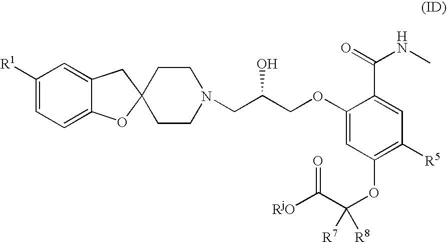

One embodiment relates to compound 2-{2-Chloro-5-{[(2S)-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic acid, tert-butyl ester.

Another embodiment relates to a compound of formula IE, or a salt thereof, where R¹ to R⁸ are defined as in formula I.

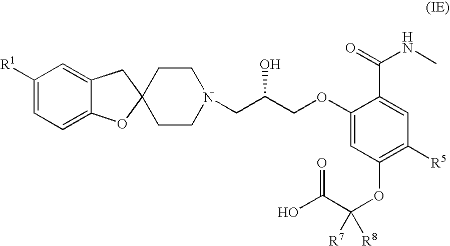

The invention further relates to the use of the intermediates in the preparation of compounds of formula I.

One embodiment relates to the use of compounds of formula (III), (V), (VI), (IX), (XI), (XXXI), (XXXII), (XXXIII), (XXXIV), (XXXV), (XXXVI), (XXXVII), (XXXVIII), (ID), (XXXIX), (XXXX), (XXI), (IE) and salts thereof, or compounds selected from
4-(5-Chloro-2-methoxybenzyl)-4-hydroxypiperidine-1-carboxylic acid, tert-butyl ester,
5-Chloro-2-hydroxy-4-(4-methoxybenzyloxy)-N-methylbenzamide,
5-Chloro-4-(4-methoxy-benzyloxy)-N-methyl-2-((S)-1-oxiranylmethoxy)benzamide,
5-Chloro-2-{[(2S)-3-(5-chloro-3H-spiro[1-benzofuran-2,4′-piperidin]-1′-yl)-2-hydroxypropyl]oxy}-4-(p-methoxybenzyloxy)-N-methylbenzamide,
5-Chloro-2-{[(2S)-3-(5-chloro-3H-spiro[1-benzofuran-2,4′-piperidin]-1′-yl)-2-hydroxypropyl]oxy}-4-hydroxy-N-methylbenzamide, trifluoroacetic acid,
4-(1-tert-Butoxycarbonyl-1-methylethoxy)-5-chloro-2-hydroxybenzoic acid, methyl ester,
2-(2-Chloro-5-hydroxy-4-methylcarbamoylphenoxy)-2-methylpropionic acid, tert-butyl ester,
2-[2-Chloro-4-methylcarbamoyl-5-((S)-1-oxiranylmethoxy)-phenoxy]-2-methylpropionic acid, tert-butyl ester, and
2-{2-Chloro-5-{[(2S)-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic acid, tert-butyl ester,
   as intermediates in the preparation of compounds of formula (I) defined as defined above.

### Pharmaceutical Composition

According to one embodiment of the present invention there is provided a pharmaceutical composition comprising as active ingredient a therapeutically effective amount of the compound of the invention, or pharmaceutically acceptable salts thereof, in association with one or more pharmaceutically acceptable diluents, excipients and/or inert carriers. The active ingredients of the present invention may be administered by oral or parenteral (e.g. intravenous, subcutaneous, intramuscular or intraarticular) administration using conventional systemic dosage forms, such as tablets, capsules, pills, powders, aqueous or oily solutions or suspensions, emulsions and sterile injectable aqueous or oily solutions or suspensions. The active ingredients may also be administered topically (e.g. to the lung and/or airways) in the form of solutions, suspensions, aerosols and dry powder formulations. These dosage forms will usually include one or more pharmaceutically acceptable ingredients which may be selected, for example, from adjuvants, carriers, binders, lubricants, diluents, stabilising agents, buffering agents, emulsifying agents, viscosity-regulating agents, surfactants, preservatives, flavourings and colorants. As will be understood by those skilled in the art, the most appropriate method of administering the active ingredients is dependent on a number of factors.

The pharmaceutical compositions of the present invention may be prepared by mixing the active ingredient with a pharmaceutically acceptable adjuvant, diluent or carrier. Therefore, in a further aspect of the present invention there is provided a process for the preparation of a pharmaceutical composition, which comprises mixing a compound of formula I, or pharmaceutically acceptable salt thereof, with a pharmaceutically acceptable adjuvant, diluent or carrier.

In one embodiment of the present invention, the active ingredient of the present invention is administered by inhalation.

The active ingredient is conveniently administered via inhalation (e.g. topically to the lung and/or airways) in the form of solutions, suspensions, aerosols or dry powder formulations. Administration may be by inhalation orally or intranasally. The active ingredient is preferably adapted to be administered, from a dry powder inhaler, pressurised metered dose inhaler, or a nebuliser.

The active ingredient may be used in admixture with one or more pharmaceutically acceptable additives, diluents or carriers. Examples of suitable diluents or carriers include lactose (e.g. the monohydrate), dextran, mannitol or glucose.

Metered dose inhaler devices may be used to administer the active ingredients, dispersed in a suitable propellant and with or without additional excipients such as ethanol, a surfactant, a lubricant, an anti-oxidant or a stabilising agent. Suitable propellants include hydrocarbon, chlorofluorocarbon and hydrofluoroalkane (e.g. heptafluoroalkane) propellants, or mixtures of any such propellants. Preferred propellants are P134a and P227, each of which may be used alone or in combination with other propellants and/or surfactant and/or other excipients. Nebulised aqueous suspensions, solutions may also be employed, with or without a suitable pH and/or tonicity adjustment, either as a unit-dose or multi-dose formulations.

Dry powder inhalers may be used to administer the active ingredients, alone or in combination with a pharmaceutically acceptable carrier, in the later case either as a finely divided powder or as an ordered mixture. The dry powder inhaler may be single dose or multi-dose and may utilise a dry powder or a powder-containing capsule.

When the active ingredient is adapted to be administered, via a nebuliser it may be in the form of a nebulised aqueous suspension or solution, with or without a suitable pH or tonicity adjustment, either as a single dose or multidose device.

Metered dose inhaler, nebuliser and dry powder inhaler devices are well known and a variety of such devices are available.

In one embodiment the present invention provides a pharmaceutical product comprising, an active ingredient which is a compound of formula I, or a pharmaceutically acceptable salt thereof, formulated for inhaled administration.

In an embodiment of the present invention, the compound of formula I, or a pharmaceutically acceptable salt thereof, may be administered orally.

### Medical Use

The compounds of formula I, salts and solvates thereof, have activity as pharmaceuticals, and are believed to be potent modulators of chemokine receptor (especially CCR1 receptor) activity, and may be used in the treatment of autoimmune, inflammatory, proliferative and hyperproliferative diseases and immunologically-mediated diseases.

A compound of the invention, or a pharmaceutically acceptable salt thereof, can be used in the treatment of:
1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins; and
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes.

A pharmaceutical product comprising, in combination, a first active ingredient which is a compound of formula I, or a pharmaceutically acceptable salt thereof, as hereinbefore described, and at least one further active ingredient selected from:
a phosphodiesterase inhibitor,
a β2 adrenoceptor agonist,
an inhibitor of kinase function,
a protease inhibitor,
a steroidal glucocorticoid receptor agonist,
an anticholinergic agent, and a
a non-steroidal glucocorticoid receptor agonist.

The pharmaceutical product according to this embodiment may, for example, be a pharmaceutical composition comprising the first and further active ingredients in admixture. Alternatively, the pharmaceutical product may, for example, comprise the first and further active ingredients in separate pharmaceutical preparations suitable for simultaneous, sequential or separate administration to a patient in need thereof.

The pharmaceutical product of this embodiment is of particular use in treating respiratory diseases such as asthma, COPD or rhinitis.

Examples of a phosphodiesterase inhibitor that may be used in the pharmaceutical product according to this embodiment include a PDE4 inhibitor such as an inhibitor of the isoform PDE4D, a PDE3 inhibitor and a PDE5 inhibitor. Examples include the compounds
(Z)-3-(3,5-dichloro-4-pyridyl)-2-[4-(2-indanyloxy-5-methoxy-2-pyridyl]propenenitrile,
N-[9-amino-4-oxo-1-phenyl-3,4,6,7-tetrahydropyrrolo[3,2,1-jk][1,4]benzodiazepin-3(R)-yl]pyridine-3-carboxamide (CI-1044),
3-(benzyloxy)-1-(4-fluorobenzyl)-N-[3-(methylsulphonyl)phenyl]-1H-indole-2-carboxamide,
(1S-exo)-5-[3-(bicyclo[2.2.1]hept-2-yloxy)-4-methoxyphenyl]tetrahydro-2(1H)-pyrimidinone (Atizoram),
N-(3,5,dichloro-4-pyridinyl)-2-[1-(4-fluorobenzyl)-5-hydroxy-1H-indol-3-yl]-2-oxoacetamide (AWD-12-281),
β-[3-(cyclopentyloxy)-4-methoxyphenyl]-1,3-dihydro-1,3-dioxo-2H-isoindole-2-propanamide (CDC-801),
N-[9-methyl-4-oxo-1-phenyl-3,4,6,7-tetrahydropyrrolo[3,2,1-jk][1,4]benzodiazepin-3(R)-yl]pyridine-4-carboxamide (CI-801),
cis-[4-cyano-4-(3-cyclopentyloxy-4-methoxyphenyl)cyclohexane-1-carboxylic acid (Cilomilast),
8-amino-1,3-bis(cyclopropylmethyl)xanthine (Cipamfylline),
N-(2,5-dichloro-3-pyridinyl)-8-methoxy-5-quinolinecarboxamide (D-4418),
5-(3,5-di-tert-butyl-4-hydroxybenzylidene)-2-iminothiazolidin-4-one (Darbufelone),
2-methyl-1-[2-(1-methylethyl)pyrazolo[1,5-a]pyridin-3-yl]-1-propanone (Ibudilast),
2-(2,4-dichlorophenylcarbonyl)-3-ureidobenzofuran-6-yl methanesulphonate (Lirimilast),
(-)-(R)-5-(4-methoxy-3-propoxyphenyl)-5-methyloxazolidin-2-one (Mesopram),
(-)-cis-9-ethoxy-8-methoxy-2-methyl-1,2,3,4,4a,10b-hexahydro-6-(4-diisopropylaminocarbonylphenyl)-benzo[c][1,6]naphthyridine (Pumafentrine),
3-(cyclopropylmethoxy)-N-(3,5-dichloro-4-pyridyl)-4-(difluoromethoxy)benzamide (Roflumilast),

### the N-oxide of Roflumilast,

5,6-diethoxybenzo[b]thiophene-2-carboxylic acid (Tibenelast),
2,3,6,7-tetrahydro-2-(mesitylimino)-9,10-dimethoxy-3-methyl-4H-pyrimido[6,1-a]isoquinolin-4-one (trequinsin), and
3-[[3-(cyclopentyloxy)-4-methoxyphenyl]-methyl]-N-ethyl-8-(1-methylethyl)-3H-purine-6-amine (V-11294A).

Examples of a β₂-adrenoceptor agonist that may be used in the pharmaceutical product according to this embodiment include metaproterenol, isoproterenol, isoprenaline, albuterol, salbutamol (e.g. as sulphate), formoterol (e.g. as fumarate), salmeterol (e.g. as xinafoate), terbutaline, orciprenaline, bitolterol (e.g. as mesylate), pirbuterol or indacaterol. The β₂-adrenoceptor agonist of this embodiment may be a long-acting β₂-agonists, for example salmeterol (e.g. as xinafoate), formoterol (e.g. as fumarate), bambuterol (e.g. as hydrochloride), carmoterol (TA 2005, chemically identified as 2(1H)-Quinolone, 8-hydroxy-5-[1-hydroxy-2-[[2-(4-methoxy-phenyl)-1-methylethyl]-amino]ethyl]-monohydrochloride, [R—(R*,R*)] also identified by Chemical Abstract Service Registry Number 137888-11-0 and disclosed in U.S. Pat. No. 4,579,854), indacaterol (CAS no 312753-06-3; QAB-149), formanilide derivatives e.g. 3-(4-{[6-({(2R)-2-[3-(formylamino)-4-hydroxyphenyl]-2-hydroxyethyl}amino)hexyl]oxy}-butyl)-benzenesulfonamide as disclosed in WO 2002/76933, benzenesulfonamide derivatives e.g. 3-(4-{[6-({(2R)-2-hydroxy-2-[4-hydroxy-3-(hydroxy-methyl)phenyl]ethyl}amino)-hexyl]oxy}butyl)benzenesulfonamide as disclosed in WO 2002/88167, aryl aniline receptor agonists as disclosed in WO 2003/042164 and WO 2005/025555, indole derivatives as disclosed in WO 2004/032921 and US 2005/222144, and compounds GSK 159797, GSK 159802, GSK 597901, GSK 642444 and GSK 678007.

Examples of an inhibitor of kinase function that may be used in the pharmaceutical product according to this embodiment include a p38 kinase inhibitor and an IKK inhibitor.

Examples of a protease inhibitor that may be used in the pharmaceutical product according to this embodiment include an inhibitor of neutrophil elastase or an inhibitor of matrix metalloproteases such as MMP1, MMP2, MMP7, MMP8, MMP9, MMP12 and/or MMP13.

Examples of a steroidal glucocorticoid receptor agonist that may be used in the pharmaceutical product according to this embodiment include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (as e.g. dicloacetate), triamcinolone (e.g. as acetonide), flunisolide, zoticasone, flumoxonide, rofleponide, butixocort (e.g. as propionate ester), prednisolone, prednisone, tipredane, steroid esters e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3 S-yl) ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters according to DE 4129535, steroids according to WO 2002/00679, WO 2005/041980, or steroids GSK 870086, GSK 685698 and GSK 799943.

Examples of an anticholinergic agent that may be used in the pharmaceutical product according to this embodiment include for example a muscarinic receptor antagonist (for example a M1, M2 or M3 antagonist, such as a M3 antagonist) for example ipratropium (e.g. as bromide), tiotropium (e.g. as bromide), oxitropium (e.g. as bromide), tolterodine, pirenzepine, telenzepine, glycopyrronium bromide (such as R,R-glycopyrronium bromide or a mixture of R,S- and S,R-glycopyrronium bromide); mepensolate (e.g. as bromide), a quinuclidine derivative such as 3(R)-(2-hydroxy-2,2-dithien-2-ylacetoxy)-1-(3-phenoxypropyl)-1-azonia-bicyclo[2.2.2]octane bromide as disclosed in US 2003/0055080, quinuclidine derivatives as disclosed in WO 2003/087096 and WO 2005/115467 and DE 10050995; or GSK 656398 or GSK 961081.

Examples of a modulator of a non-steroidal glucocorticoid receptor agonist that may be used in the pharmaceutical product according to this embodiment include those described in WO2006/046916.

One embodiment of the present invention provides a compound of formula I or a pharmaceutically acceptable salt thereof, as hereinbefore defined for use in therapy.

Another embodiment of the present invention provides the use of a compound of formula I or a pharmaceutically acceptable salt thereof, as hereinbefore defined in the manufacture of a medicament for the treatment of human diseases or conditions in which modulation of CCR1 activity is beneficial.

A further embodiment of the present invention provides the use of a compound of formula I or a pharmaceutically acceptable salt thereof, as hereinbefore defined in the manufacture of a medicament for use in treating a respiratory disease.

Yet another embodiment of the present invention provides the use of a compound of formula I or a pharmaceutically acceptable salt thereof, as hereinbefore defined in the manufacture of a medicament for use in treating an airways disease.

Yet a further embodiment of present invention provides the use of a compound of formula I or a pharmaceutically acceptable salt thereof, as hereinbefore defined in the manufacture of a medicament for use in treating an inflammatory disease.

One embodiment of the present invention provides the use of a compound of formula I or a pharmaceutically acceptable salt thereof, as hereinbefore defined in the manufacture of a medicament for use in treating chronic obstructive pulmonary disease (COPD).

Another embodiment of the present invention provides the use of a compound of formula I or a pharmaceutically acceptable salt thereof, as hereinbefore defined in the manufacture of a medicament for use in treating asthma.

A further embodiment of the present invention provides a method of treatment of respiratory diseases, airway diseases, inflammatory diseases, COPD and/or asthma, in a patient suffering from, or at risk of, said disease, which comprises administering to the patient a therapeutically effective amount of a compound of formula I, or a pharmaceutically acceptable salt thereof, as hereinbefore defined.

Another embodiment of the present invention provides the method above whereby the compound of formula I or a pharmaceutically acceptable salt thereof, as defined above is administered by inhalation.

One embodiment of the invention relates to an agent for the treatment of respiratory diseases, airway diseases, inflammatory diseases, COPD and/or asthma, which comprises as active ingredient a compound of formula I or a pharmaceutically acceptable salt thereof.

Another embodiment relates to the use of a pharmaceutical composition comprising the compound of formula I or a pharmaceutically acceptable salt thereof in the treatment of respiratory diseases, airway diseases, inflammatory diseases, COPD and/or asthma.

In the context of the present specification, the term “therapy” also includes “prophylaxis” unless there are specific indications to the contrary. The terms “therapeutic” and “therapeutically” should be construed accordingly.

In this specification, unless stated otherwise, the terms “inhibitor” and “antagonist” mean a compound that by any means, partly or completely, blocks the transduction pathway leading to the production of a response by the agonist.

The term “disorder”, unless stated otherwise, means any condition and disease associated with CCR1 receptor activity.

For the above-mentioned therapeutic uses the dosage administered will, of course, vary with the compound employed, the mode of administration, the treatment desired and the disorder indicated. The daily dosage of the compound of formula I may be in the range from 0.1 μg/kg to 30 mg/kg.

The compound of formula I or pharmaceutically acceptable salt thereof may be used on their own but will generally be administered in the form of a pharmaceutical composition in which the formula I compound/salt/solvate (active ingredient) is in association with a pharmaceutically acceptable adjuvants, diluents and/or carriers. Depending on the mode of administration, the pharmaceutical composition will preferably comprise from 0.01 to 100% w (percent by weight), more preferably from 0.01 to 80% w, still more preferably from 0.05 to 70% w, and even more preferably from 0.05 to 50% w, of active ingredient, all percentages by weight being based on total composition.

### EXAMPLES

The invention will now be further explained by reference to the following illustrative examples.

Each exemplified compound represents a particular and independent aspect of the invention.

The following abbreviations are used:

### APCI-MS Atmospheric Pressure Chemical Ionisation Mass Spectroscopy;

DBU 1,8-Diazabicyclo[5.4.0]undec-7-ene

### DCM Dichloromethane

### DIEA N,N-Diisopropylethylamine;

### DME 1,2-Dimethoxyethane

### DMF N,N-Dimethylformamide

### DMSO Dimethylsulfoxide;

### HPLC High Performance Liquid Chromatography;

### LC/MS Liquid Column Chromatography/Mass Spectroscopy;

NMP N-methyl-2-pyrrolidone
ns 3-nitrobenzenesulphonyl
PMB p-methoxybenzyl
PrCN n-Butyronitrile
TBME tert-Butylmethyl ether
TFA Trifluoroacetic acid;

### THF Tetrahydrofuran

BOC tert-butocycarbonyl
Rel vol relative volume

### General Methods

¹H NMR and ¹³C NMR spectra were recorded on a Varian Inova 400 MHz or a Varian Mercury-VX 300 MHz or a Varian Unity Inova 400 MHz or a Varian Unity Inova 300 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Flash chromatography was carried out using silica gel (0.040-0.063 mm, Merck). Unless stated otherwise, starting materials were commercially available. All solvents and commercial reagents were of laboratory grade and were used as received.

The following methods were used for LC/MS analysis:

Instrument Agilent 1100; Column Waters Symmetry 2.1×30 mm; Mass APCI; Flow rate 0.7 ml/min; Wavelength 254 nm; Solvent A: water+0.1% TFA; Solvent B: acetonitrile+0.1% TFA; Gradient 15-95%/B 2.7 min 95% B 0.3 min.
Instrument Agilent 1100; Column Hi Chrom Ace Phenyl 3.0×50 mm; Mass APCI; Flow rate 1.25 ml/min; Wavelength 230 nm; Solvent A: water+0.03% TFA; Solvent B: acetonitrile+0.03% TFA; Gradient 5-95% B 6 min, 95% B 1.5 min.

The following method was used for LC analysis:

Method A. Instrument Agilent 1100; Column: Kromasil C18 100×3 mm, 5μ particle size,
Solvent A: 0.1% TFA/water, Solvent B: 0.08% TFA/acetonitrile Flow: 1 ml/min,
Gradient 10-100% B 20 min, 100% B 1 min. Absorption was measured at 220, 254 and 280 nm.
Method B. Instrument Agilent 1100; Column: XTerra C8, 100×3 mm, 5μ particle size,
Solvent A: 15 mM NH₃/water, Solvent B: acetonitrile Flow: 1 ml/min, Gradient 10-100% B 20 min, 100% B 1 min. Absorption was measured at 220, 254 and 280 nm.

The following intermediates and starting materials can be prepared following the procedures described in WO2004005295:
5-chloro-3H-spiro[1-benzofuran-2,4′-piperidine],
5-fluoro-3H-spiro[1-benzofuran-2,4′-piperidine],
5-chloro-2-hydroxy-4-[(4-methoxybenzyl)oxy]-N-methylbenzamide,
(3S)-1-(5-chloro-2-{[(2S)-3-(5-chloro-1′H,3H-spiro[1-benzofuran-2,4′-piperidin]-1′-yl)-2-hydroxypropyl]oxy}-4-hydroxybenzoyl)pyrrolidin-3-ol,
N-[5-chloro-2-[(2S)-3-(5-chlorospiro[benzofuran-2(3H), 4′-piperidin]-1′-yl)-2-hydroxypropoxy]-4-methoxyphenyl]acetamide,
N-[2-[(2S)-3-(5-chlorospiro[benzofuran-2(3H), 4′-piperidin]-1′-yl)-2-hydroxypropoxy]-4-hydroxyphenyl]acetamide,
N-{5-chloro-4-methoxy-2-[(2S)-oxiran-2-ylmethoxy]phenyl}acetamide,

The following intermediates and starting materials can be prepared following procedures similar to those described in WO2000012468,
[5-chloro-2-hydroxy-4-(4-methoxy-benzyloxy)-phenyl]carbamic acid tertbutyl ester,

The following intermediates and starting materials can be prepared following procedures similar to those described in WO2001077101:
1,4′-Bipiperidine, 4-(2,4-dichloro-3-methylphenoxy)-1′-[4-(methylsulfonyl)benzoyl],

### Example 1

### (4-(acetylamino)-2-chloro-5-{[(2S)-3-(5-chloro-1′H, 3H-spiro[1-benzofuran-2,4′-piperidin]-1′-yl)-2-hydroxypropyl]oxy}phenoxy)acetic Acid

### Step 1: Tert-Butyl {5-chloro-4-[(4-methoxybenzyl)oxy]-2-[(2S)-oxiran-2-ylmethoxy]phenyl}carbamate

To a solution of tert-butyl {5-chloro-2-hydroxy-4-[(4-methoxybenzyl)oxy]phenyl}carbamate (2.9 g) in NMP (20 ml) was added cesium carbonate (2.6 g) and (2S)-oxiran-2-ylmethyl 3-nitrobenzenesulfonate (1 eq). The reaction was stirred at room temperature for 18 h and then partitioned between diethyl ether and water. The organics were dried over sodium sulfate and the solvent removed to yield 3.3 g (99%) of the subtitled compound as identified by APCI-MS (m/z 435 (M⁺)) as a mobile oil.

### Step 2: Methyl (4-amino-2-chloro-5-{[(2S)-3-(5-chloro-1′H,3H-spiro[1-benzofuran-2,4′-piperidin]-1′-yl)-2-hydroxypropyl]oxy}phenoxy)acetate

To a solution of tert-butyl {5-chloro-4-[(4-methoxybenzyl)oxy]-2-[(2S)-oxiran-2-ylmethoxy]phenyl}carbamate (1 g) in ethanol (10 ml) 5-chloro-3H-spiro[1-benzofuran-2,4′-piperidine] (0.51 g) added. The reaction was heated at 80° C. for 1 h and then concentrated in vacuo and the residue redissolved in dichloromethane (2 ml) to which 1 M HCl in diethyl ether (5 ml) was added. The mixture was stirred for at room temperature for 24 h, and concentrated in vacuo. The residue was subjected to column chromatography starting gradient 1:1 EtOAc:iHex to elute fast running impurities, and finally eluting 220 mg of the subtitled compound as identified by APCI-MS (m/z 539 (M⁺)) as an off-white solid with 10% methanol in dichlormethane.

### Step 3: Methyl (4-amino-2-chloro-5-{[(2S)-3-(5-chloro-1′H,3H-spiro[1-benzofuran-2,4′-piperidin]-1′-yl)-2-hydroxypropyl]oxy}phenoxy)acetate

To a solution of methyl (4-amino-2-chloro-5-{[(2S)-3-(5-chloro-1′H,3H-spiro[1-benzofuran-2,4′-piperidin]-1′-yl)-2-hydroxypropyl]oxy}phenoxy)acetate (220 mg) in NMP (5 ml) cesium carbonate (132 mg) and methyl bromoacetate (60 mg) were added. The reaction was heated at 80° C. for 30 min then cooled and partitioned between diethyl ether and water; the organics were dried over sodium sulfate and concentrated in vacuo. The residue was redissolved in dichloromethane (5 ml) and trifluoroacetic acid (1 ml) was added. The reaction was stirred for 18 h, and then concentrated in vacuo. The residue was partitioned then between dichloromethane and a saturated sodium bicarbonate solution (aq). The organic layer was dried over sodium sulfate and concentrated in vacuo to give 140 mg of the subtitled compound as identified by APCI-MS (m/z 511 (M⁺)) as an off-white gum.

### Step 4: (4-(Acetylamino)-2-chloro-5-{[(2S)-3-(5-chloro-1′H,3H-spiro[1-benzofuran-2,4′-piperidin]-1′-yl)-2-hydroxypropyl]oxy}phenoxy)acetic Acid

To a solution of methyl (4-amino-2-chloro-5-{[(2S)-3-(5-chloro-1′H,3H-spiro[1-benzofuran-2,4′-piperidin]-1′-yl)-2-hydroxypropyl]oxy}phenoxy)acetate (0.14 g) in dichloromethane (5 ml) Hunigs' base (70 ul) and acetic anhydride (31 ul) were added. After 30 min the reaction was concentrated in vacuo and the residue redissolved in 5 ml of 1:1 THF:water. LiOH (20 mg) was added and the mixture was stirred at room temperature for 18 h. The solvents were removed in vacuo and the residue subjected to RPHPLC using two different systems (Xterra column first using 5% to 75% acetonitrile in aq NH₃ (0.2%) then 25% to 95% acetonitrile in aq NH₄OAc (0.2%) giving 15 mg (10%) of the title compound as a white solid.

1H NMR (DMSO) δ 8.97 (s, H), 7.93 (s, H), 7.24 (s, H), 7.11 (d, H), 6.75 (d, H), 6.59 (s, H), 4.57 (s, 2H), 4.14-4.05 (m, H), 3.92-3.80 (m, 2H), 3.54-3.34 (m, 2H), 3.02 (s, 2H), 2.90-2.64 (m, 4H), 2.06 (s, 3H), 1.93-1.79 (m, 4H); APCI-MS: m/z 537 (M).

Result from assay: pIC50 9.25.

### Example 2

### (4-(acetylamino)-3-{[(2S-3-(5-chloro-1′H,3H-spiro[1-benzofuran-2,4′-piperidin]-1′-yl)-2-hydroxypropyl]oxy}phenoxy)acetic Acid

### Method A.

### Step 1: Methyl (4-acetylamino-5-{[(2S)-3-(5-chloro-1′H,3H-spiro[1-benzofuran-2,4′-piperidin]-1′-yl)-2-hydroxypropyl]oxy}phenoxy)acetate

To a solution of N-[2-[(2S)-3-(5-chlorospiro[benzofuran-2(3H), 4′-piperidin]-1′-yl)-2-hydroxypropoxy]-4-hydroxyphenyl]acetamide (45 mg) in DMF (1 ml) cesium carbonate (49 mg) and methyl bromoacetate (15 mg) were added. The reaction was stirred at room temperature for 6 h, after which the mixture was filtered and purified by reverse HPLC (water:acetonitrile containing 1% TFA) yielding 21 mg (33%) of the subtitled compound as a white solid.

¹H-NMR (d6-DMSO) δ 9.57-9.48 (m, H), 8.94 (d, J=8.3, NH), 7.67-7.64 (m, H), 7.31-7.29 (m, H), 7.17-7.15 (m, H), 6.82-6.78 (m, H), 6.69-6.68 (m, H), 6.51-6.48 (m, H), 6.03 (b, OH), 4.78 (s, 3H), 4.34-4.27 (m, 1H), 4.04-3.89 (m, 2H), 3.70 (s, 3H), 3.68-3.15 (m, 6H), 3.11 (s, 2H), 2.20-2.00 (m, 4H), 2.07 (s, 3H); APCI-MS: m/z 519 (MH⁺).

### Step 2: 5-{[(2S)-3-(5-chloro-1′H,3H-spiro[1-benzofuran-2,4′-piperidin]-1′-yl)-2-hydroxypropyl]oxy}-4-{acetylamino}phenoxy)acetic Acid

A mixture of methyl (4-acetylamino-5-{[(2S)-3-(5-chloro-1′H,3H-spiro[1-benzofuran-2,4′-piperidin]-1′-yl)-2-hydroxypropyl]oxy}phenoxy)acetate (43 mg) in ethanol (2 ml) and 2 M NaOH (aq) (1 ml) is heated for 1 h, after which the solvents are removed and the compound purified by reverse HPLC (water:acetonitrile containing 1% TFA) yielding 42 mg (87%) of the TFA salt of the titled compound as a white solid.

¹H-NMR (d6-DMSO)) 9.57-9.48 (m, H), 8.94 (d, J=8.3, NH), 7.65-7.63 (m, H), 7.29 (m, H), 7.17-7.15 (m, H), 6.82-6.78 (m, H), 6.67-6.66 (m, H), 6.49-6.46 (m, H), 6.02 (b, OH), 4.66 (s, 2H), 4.40-4.29 (m, H), 4.02-3.92 (m, 2H), 3.56-3.19 (m, 6H), 3.10 (s, 2H), 2.19-2.00 (m, 4H), 2.07 (s, 3H); APCI-MS: m/z 505 (MH⁺).

### Method B

### 5-{[(2S)-3-(5-chloro-1′H,3H-spiro[1-benzofuran-2,4′-piperidin]-1′-yl)-2-hydroxypropyl]oxy}-4-{acetylamino}phenoxy)acetic Acid

To a solution of N-[2-[(2S)-3-(5-chlorospiro[benzofuran-2(3H), 4′-piperidin]-1′-yl)-2-hydroxypropoxy]-4-hydroxyphenyl]acetamide, trifluoroacetic acid salt (145 mg) in THF (3 ml) was added sodium hydride (60% in oil; 13 mg). The mixture was stirred for 30 min whereupon methyl bromoacetate (50 mg) was added. The reaction was stirred at room temperature for 20 h. Then methanol (3 ml) and lithium hydroxide (0.1 g) were added and the mixture was heated at 50° C. for 2 h, cooled, concentrated in vacuo and purified by reverse phase HPLC (Xterra, ammonia: acetonitrile) leaving 13 mg (8%) of the subtitled compound as a white solid.

¹H-NMR (d6-DMSO) 8.89 (s, H), 7.63 (d, H), 7.22 (d, H), 7.08 (dd, H), 6.74 (d, H), 6.50 (d, H), 6.33 (dd, H), 5.10 (s, H), 4.03 (s, 2H), 4.05-3.90 (m, H), 3.80 (dd, H), 3.00 (s, 2H), 2.70-2.40 (m, 6H), 2.04 (s, 3H), 1.9-1.6 (m, 4H); APCI-MS: m/z 505 (MH⁺).

Result from assay: IC50 (μM) 0.01003.

### Example 3

### (4-(acetylamino)-2-chloro-5-{[(2S)-3-(5-fluoro-1′H,3H-spiro[1-benzofuran-2,4′-piperidin]-1′-yl)-2-hydroxypropyl]oxy}phenoxy)acetic Acid, Hydrochloride

### Step 1. N-(5-Chloro-2-{[(2S)-3-(5-fluoro-1′H, 3H-spiro[1-benzofuran-2,4′-piperidin]-1′-yl)-2-hydroxypropyl]oxy}-4-hydroxyphenyl)acetamide

A solution of N-{5-chloro-4-methoxy-2-[(2S)-oxiran-2-ylmethoxy]phenyl}acetamide (1 g) and 5-fluoro-3H-spiro[1-benzofuran-2,4′-piperidine] (0.75 g) in ethanol (20 ml) was heated at 80° C. for 1 h and concentrated in vacuo. The residue was redissolved in dichloromethane and boron tribromide (1.0 M soln in dichloromethane, 3.6 ml) was added dropwise. The reaction was stirred at 30° C. for 1 h. After which second portion of BBr₃ (1.0 M soln in dichloromethane, 3.6 ml) was added. The mixture was stirred at 30° C. for 18 h. The reaction was quenched with methanol (20 ml) and concentrated in vacuo. The residue was redissolved in dichloromethane (10 ml) and a fresh portion of BBr₃ (1.0 M soln in dichloromethane, 3.6 ml) added. The reaction mixture is heated at reflux for 1 h. Then methanol (10 ml) is added and the reaction heated at reflux again for 1 h, the solvent is removed in vacuo. The residue was partitioned between dichloromethane and a 10% NaOH (aq) solution. The aqueous phase was acidified with a 10% HCl solution and extracted with ethylacetate. The organics were dried over sodium sulfate and concentrated in vacuo to give 380 mg (xx %) of the subtitled compound, as identified by APCI-MS (m/z 464 (M⁺)) as an off-white, gummy solid.

### Step 2. (4-(acetylamino)-2-chloro-5-{[(25S-3-(5-fluoro-1′H,3H-spiro[1-benzofuran-2,4′-piperidin]-1′-yl)-2-hydroxypropyl]oxy}phenoxy)acetic Acid, Hydrochloric Acid Salt

To a solution of N-(5-chloro-2-{[(2S)-3-(5-fluoro-1′H,3H-spiro[1-benzofuran-2,4′-piperidin]-1′-yl)-2-hydroxypropyl]oxy}-4-hydroxyphenyl)acetamide (0.18 g) in DMF (5 ml) cesium carbonate (0.12 g) and methyl bromoacetate (30 μl) were added. The mixture was heated at 60° C. for 8 h. The reaction was partitioned between EtOAc and water and the organics were dried over sodium sulfate and removed in vacuo. The residue was taken up in 1:1 THF:water and LiOH (20 mg) was added. The reaction was stirred at room temperature for 60 min after which it was concentrated in vacuo and subjected to RPHPLC (Xterra, 5% to 50% acetonitrile in aq. NH₃ (0.2%)). Treatment with HCl in ether gave 8 mg of the titled compound, as identified by APCI-MS (m/z 521 (M—H)) as an off-white solid.

Result from assay: pIC50 8.65.

### Example 4

### {2-Chloro-5-{[(2S)-3-(5-chloro-1′H,3H-spiro[1-benzofuran-2,4′-piperidin]-1′-yl)-2-hydroxyropyl]oxy}-4-[methylamino)carbonyl]phenoxy}acetic Acid

### Method A

### Step 1: 5-Chloro-2-hydroxy-4-methoxy Benzoic Acid

Methyl 2-hydroxy-4-methoxybenzoate (50.0 g) in dichloromethane (250 ml) and aqueous HCl (5.0 ml of 2.5 M) was treated with SO₂Cl₂ (38.9 g) and the mixture was heated to reflux for 2 h. The mixture was allowed to cool to ambient temperature and the solvent was evaporated to give a white solid which was slurried in methanol (250 ml) for 30 min and then filtered. The filter cake was washed with methanol (100 ml) and then dried to constant mass in a vacuum oven to give the subtitled compound, as identified by LC-MS, as a white solid (49.0 g, 82% yield).

### Step 2: 5-chloro-2,4-dihydroxy Benzoic Acid

Methyl 2-hydroxy-4-methoxy-5-chlorobenzoate (49.0 g) was suspended in dodecane thiol (250 ml) and heated to 40° C. The slurry was then treated with aluminium trichloride (75.4 g) over 5 min. The mixture was then stirred for a further 1 h at 40° C. Ice was then added to quench the reaction and then the mixture was partitioned between water (200 ml) and ethyl acetate (400 ml). The organic extracts were dried and the ethyl acetate removed in vacuo. The resulting solution in dodecanethiol was allowed to cool to ambient temperature and stirred overnight. The resulting slurry was filtered and the filter cake was washed with isohexane (750 ml) to give the subtitled compound, as identified by LC-MS, as a white solid (31.2 g, 68% yield).

### Step 3: 5-Chloro-2-hydroxy-4-[(4-methoxybenzyl)oxy]-N-methylbenzamide

Methyl 2,4-dihydroxy-5-chlorobenzoate (31.2 g) in dry DMF (90 ml) was treated with potassium carbonate (23.41 g) and then 4-methoxybenzyl chloride (24.12 g). The mixture was heated to 65° C. for 18 h and then allowed to cool to room temperature. Water (100 ml) was added and the mixture stirred for 1 h. The resulting slurry was filtered and the filter cake washed with water (50 ml). The damp solid was suspended in methanol (300 ml, 10 vol) and stirred for 30 min. The slurry was filtered and the filter cake was washed with methanol (90 ml) and dried yielding 33.0 g (66%) of methyl 2-hydroxy-4-[(4-methoxybenzyl)oxy]-5-chlorobenzoate, which was converted to the subtitled compound as described in WO2004005295.

### Step 4: 5-Chloro-2{[(4R)-2,2-dimethyl-1,3-dioxolan-4-yl]methoxy}-4-[(4-methoxybenzyl]oxy-N-methylbenzamide

A slurry of 5-chloro-2-hydroxy-4-[(4-methoxybenzyl)oxy]-N-methylbenzamide (3.2 g), D-α,β-isoprpylidenglycerol-γ-tosylate (2.9 g) and cesium carbonate (3.6 g) in DMF (10 ml) was heated to 100° C. for 3 h. The mixture was extracted between ethyl acetate (200 ml) and water (100 ml). The organic layer was washed 3 times with water (100 ml), dried and removed in vacuo yielding 4.2 g (95.6%) of the subtitled compound as a white solid.

¹H-NMR (acetone-d6, 400 MHz): δ 8.11 (s, 1H), 7.45 (d, J=8.9, 2H), 7.01 (s, 1H), 6.97 (d, J=8.8, 2H), 5.23 (s, 2H), 4.66-4.60 (m, 1H), 4.64-4.43 (m, 1H), 4.24-4.01 (m, 3H), 3.81 (s, 3H), 2.87 (d, J=4.6, 3H), 1.44 (s, 3H), 1.36 (s, 3H); APCI-MS: m/z 436 (MH⁺).

### Step 5: 5-Chloro-4-(4-hydroxoxy)-N-methyl-2-[(2S)-oxiran-2-ylmethoxy]benzamide

To a warmed, 30° C., solution of 5-chloro-2{[(4R)-2,2-dimethyl-1,3-dioxolan-4-yl]methoxy}-4-[(4-methoxybenzyl]oxy-N-methylbenzamide (0.5 g) in acetic acid (5 ml) is added dropwise a 4.1 M hydrobromic acid in acetic acid solution (1 ml). The reaction mixture is stirred at 30° C. for 90 min, when a quick precipitation occurres. The reaction is quenched with 2 N sodium hydroxide (aq; 18 ml, pH ˜5.5) and extracted with ethyl acetate. The combined organic layers are dried and removed in vacuo. The intermediate is redissolved in methanol (10 ml) and a 0.5 M solution of sodium methoxide in methanol (4.8 ml) is added. The mixture is stirred at room temperature for 20 min, after which the reaction is quenched by adding conc. acetic acid to pH 6-7. The solvent is evaporated and the residue redissolved in ethyl acetate (100 ml) and washed with water (4×25 ml). The organic layer is dried and removed in vacuo yielding 257 (87%) of the subtitled compound as a white solid.

¹H-NMR (dmso-d6, 400 MHz): δ 7.89 (d, J=4.6, 1H), 7.74 (s, 1H), 6.68 (s, 1H), 4.46-4.41 (m, 1H), 4.01-3.95 (m, 1H), 3.47-3.42 (m, 1H), 2.90-2.87 (m, 1H), 2.79 (d, J=4.8, 3H), 2.78-2.76 (m, 1H); APCI-MS: m/z 259 (MH⁺).

### Step 6: 5-Chloro-2-{[(2S)-3-(5-chloro-1′H,3H-spiro[1-benzofuran-2,4′-piperidin]-1′-yl)-2-hydroxypropyl]oxy}-4-hydroxy-N-methylbenzamide

A suspension of 5-chloro-3H-spiro[1-benzofuran-2,4′-piperidine] (223 mg), 5-chloro-4-(4-hydroxoxy)-N-methyl-2-[(2S)-oxiran-2-ylmethoxy]benzamide (257 mg) and lithium perchlorate (10 mg) in acetonitrile (5 ml) is heated to reflux for 4 h. Some methanol is added to the mixture to dissolve solids and the reaction mixture is purified over reversed HPLC using a gradient of water:acetonitrile containing 1% TFA, yielding 350 mg (58%) of the TFA salt of the subtitled compound as a white solid.

¹H-NMR (DMSO-d6, 400 MHz): δ 8.05 (d, J=4.6, NH), 7.73 (s, 1H), 7.30 (m, 1H), 7.18-7.14 (m, 1H), 6.82-6.78 (m, 1H), 6.73 (s, 1H), 4.42 (m, 1H), 4.05 (s, 2H), 3.57 (m, 2H), 3.45-3.40 (m, 1H), 3.27-3.11 (m, 5H), 2.81 (d, J=4.8, 3H), 2.18-2.08 (m, 4H); APCI-MS: m/z 481 (MH⁺).

### Step 7: Methyl{2-chloro-5-{[(2S)-3-(5-chloro-1′H, 3H-spiro[1-benzofuran-2,4′-piperidin]-1′-yl)-2-hydroxypropyl]oxy}-4-[(methylamino)carbonyl]phenoxy}acetate

To a stirred solution of 5-chloro-2-{[(2S)-3-(5-chloro-1′H,3H-spiro[1-benzofuran-2,4′-piperidin]-1′-yl)-2-hydroxypropyl]oxy}-4-hydroxy-N-methylbenzamide, TFA salt, (119 mg, 0.2 mmol) in DMF (2.5 ml) were added cesium carbonate (163 mg, 0.5 mmol) and methyl bromoacetate (30 mg, 0.2 mmol). After stirring at room temperature for 2 h the inorganic material was removed by filtration. The product was isolated by HPLC to afford the subtitle compound as colourless solid (TFA salt, 91 mg, 68%).

¹H-NMR (acetone-d₆, 400 MHz): δ 7.99 (s, 1H), 7.23 (s, 1H), 7.13 (dd, J=8.5, 2.2 Hz, 1H), 6.91 (s, 1H), 6.76 (d, J=8.6 Hz, 1H), 4.96 (s, 2H), 4.71 (m, 1H), 4.29 (m, 2H), 3.85 (br.s, 1H), 3.75 (s, 3H), 3.60 (m, 1H), 3.50 (dd, J=13.3, 9.9 Hz, 2H), 3.18 (br.s, 2H), 3.04 (br.s, 4H), 2.90 (d, J=3.8 Hz, 3H), 2.46-2.15 (m, 4H); APCI-MS: m/z 553 (MH⁺).

### Step 8: {2-Chloro-5-{[(2S)-3-(5-chloro-1H,3H-spiro[1-benzofuran-2,4′-piperidin]-1′-yl)-2-hydroxypropyl]oxy}-4-[(methylamino)carbonyl]phenoxy}acetic Acid

To a solution of methyl {2-chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}acetate, TFA salt, (67 mg, 0.1 mmol) in ethanol (5 ml) was added aq. NaOH (2 M, 1 ml). The mixture was heated at 80° C. for 1 h. the volatiles were removed in vacuo. The residue was purified by HPLC to give the title compound as colourless solid (TFA salt, 48 mg, 74%).

¹H-NMR (acetone-d₆, 400 MHz): δ 7.97 (s, 1H), 7.23 (s, 1H), 7.13 (dd, J=8.5, 2.2 Hz, 1H), 6.91 (s, 1H), 6.76 (d, J=8.6 Hz, 1H), 4.93 (s, 2H), 4.71 (m, 1H), 4.31 (m, 2H), 3.79 (s, 2H), 3.62 (d, J=13.3 Hz, 1H), 3.50 (dd, J=13.4, 9.9 Hz, 2H), 3.32-3.12 (m, 4H), 2.89 (s, 3H), 2.43-2.19 (m, 4H); APCI-MS: m/z 539 (MH⁺).

Result from assay: IC50 (μM) 0.02019.

### Example 5

### 2-{2-Chloro-5-{[(28)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic Acid TFA

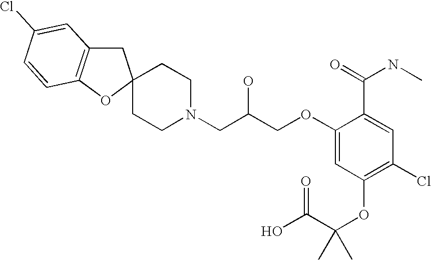

### Step 1: 5-Chloro-4-[(4-methoxybenzyl)oxy]-N-methyl-2-[(2S)-oxiran-2-ylmethoxy]benzamide

A suspension of methyl 5-chloro-2-hydroxy-4-[(4-methoxybenzyl)oxy]benzoate (1.61 g, 5.0 mmol) in the solution of methylamine in ethanol (33% wt., 25 ml) was stirred at room temperature overnight, then at 60° C. for 4 h to form a solution. The solvent was removed in vacuo, affording 5-chloro-2-hydroxy-4-[(4-methoxybenzyl)oxy]-N-methylbenzamide as red powder. This intermediate was dissolved in DMF (20 ml). To the solution were added cesium carbonate (1.96 g, 6.0 mmol) and (2)-oxiran-2-ylmethyl 3-nitrobenzenesulfonate (1.30 g, 5.0 mmol). The suspension was stirred at room temperature overnight. The reaction mixture was partitioned between ethyl acetate and H₂O. The organic layer was dried over Na₂SO₄, and filtered. The solvent was removed in vacuo to give the subtitled compound as red solid (1.71 g, 91%).

¹H-NMR (CDCl₃, 400 MHz): δ 8.21 (s, 1H), 7.67 (d, J=4.4, NH), 7.37 (d, J=8.76, 2H), 6.92 (d, J=8.76, 2H), 6.58 (s, 1H), 5.11 (s, 2H), 4.44-4.39 (m, 1H), 4.01-3.96 (m, 1H), 3.82 (s, 3H), 3.39-3.34 (m, 1H), 2.98 (d, J=4.9, 3H), 2.96-2.95 (m, 1H), 2.82-2.79 (m, 1H); APCI-MS: m/z 378 (MH⁺).

### Step 2: 5-Chloro-2-{[(2S)-3-(5-chloro-1′H,3H-spiro[1-benzofuran-2,4′-piperidin]-1′-yl)-2-hydroxypropyl]oxy}-4-hydroxy-N-methylbenzamide

A mixture of 5-chloro-3H-spiro[1-benzofuran-2,4′-piperidine] (172 mg, 0.77 mmol) and 5-chloro-4-[(4-methoxybenzyl)oxy]-N-methyl-2-[(2S)-oxiran-2-ylmethoxy]benzamide (290 mg, 0.77 mmol) in ethanol (10 ml) was stirred at 80° C. overnight. Ethanol was then removed in vacuo. The residue was redissolved in dichloromethane (5 ml). Aq. TFA (95%, 2.5 ml) was added, and the solution was stirred at room temperature for 2 h. The volatiles were removed in vacuo and the residue was purified by HPLC to give the subtitle compound as TFA salt (382 mg, 72%).

¹H-NMR (DMSO-d6, 400 MHz): δ 8.05 (d, J=4.6, NH), 7.73 (s, 1H), 7.30 (m, 1H), 7.18-7.14 (m, 1H), 6.82-6.78 (m, 1H), 6.73 (s, 1H), 4.42 (m, 1H), 4.05 (s, 2H), 3.57 (m, 2H), 3.45-3.40 (m, 1H), 3.27-3.11 (m, 5H), 2.81 (d, J=4.8, 3H), 2.18-2.08 (m, 4H); APCI-MS: m/z 481 (MH⁺).

### Step 3: Ethyl 2-{2-chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoate

To a stirred solution of 5-chloro-2-{[(2S-3-(5-chloro-1′H,3H-spiro[1-benzofuran-2,4′-piperidin]-1′-yl)-2-hydroxypropyl]oxy}-4-hydroxy-N-methylbenzamide, TFA salt, (Example 4, Step 2, 67 mg, 0.112 mmol) in DMF (1 ml) were added cesium carbonate (92 mg, 0.281 mmol) and ethyl 2-bromo-2-methylpropanoate (24 mg, 0.123 mmol). After stirring at 45° C. overnight another portion of ethyl 2-bromo-2-methylpropanoate (24 mg, 0.123 mmol) was added. The reaction mixture was stirred for an additional 4 h. The reaction mixture was partitioned between ethyl acetate and H₂O. The organic layer was washed with brine, and dried with sodium sulfate. After evaporation of solvent the product was isolated by HPLC to afford the subtitle compound, as identified by APCI-MS (m/z 595 (MH⁺)), as colourless solid (TFA salt, 68 mg, 86%).

### Step 4: 2-{2-Chloro-5-{[(2S)-3-(5-chloro-1′H, 3H-spiro[1-benzofuran-2,4′-piperidin]-1′ yl)-2-hydroxypropyl]-oxy}-4-(methylamino)carbonyl]phenoxy}-2-methylpropanoic Acid

To a solution of ethyl 2-{2-chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoate (33 mg, 55 μmol) in dioxane (2 ml) were added aq. NaOH (55 μl), and water (0.5 ml). The mixture was heated at 80° C. for 30 min. Then it was acidified with aq. HCl (2 M, 200 μl), and concentrated. The product was isolated by HPLC to give the title compound as colourless oil (TFA salt, 17 mg, 45%).

¹H-NMR (DMSO-d₆, 400 MHz): δ 13.41 (br.s, 1H), 9.60-9.35 (m, 1H), 8.13 (d, J=4.6 Hz, 1H), 7.75 (s, 1H), 7.30 (s, 1H), 7.16 (d, J=8.7 Hz, 1H), 6.80 (d, J=8.5 Hz, 1H), 6.19 (s, 1H), 4.40 (br.s, 1H), 4.00 (d, J=4.4 Hz, 2H), 3.62-3.15 (m, 6H), 3.11 (s, 2H), 2.82 (d, J=4.7 Hz, 3H), 2.50 (m, 4H), 1.60 (s, 6H); APCI-MS: m/z 567 (MH⁺).

Result from assay: IC50 (μM) 0.001057.

### Example 6

### R-Enantiomer

### 2-{2-Chloro-5-{[(2R)-3-(5-chloro-1′H,3H-spiro[1-benzofuran-2,4′-piperidin]-1′-yl-2-hydroxyropyl]oxy}-4-[(methylamino)carbonyl]-phenoxy}-2-methylpropanoic Acid

This compound was prepared using the same process as described in example 5 but using 5-chloro-4-[(4-methoxybenzyl)oxy]-N-methyl-2-[(2R)-oxiran-2-ylmethoxy]benzamide.

APCI-MS: m/z 567 (MH⁺).

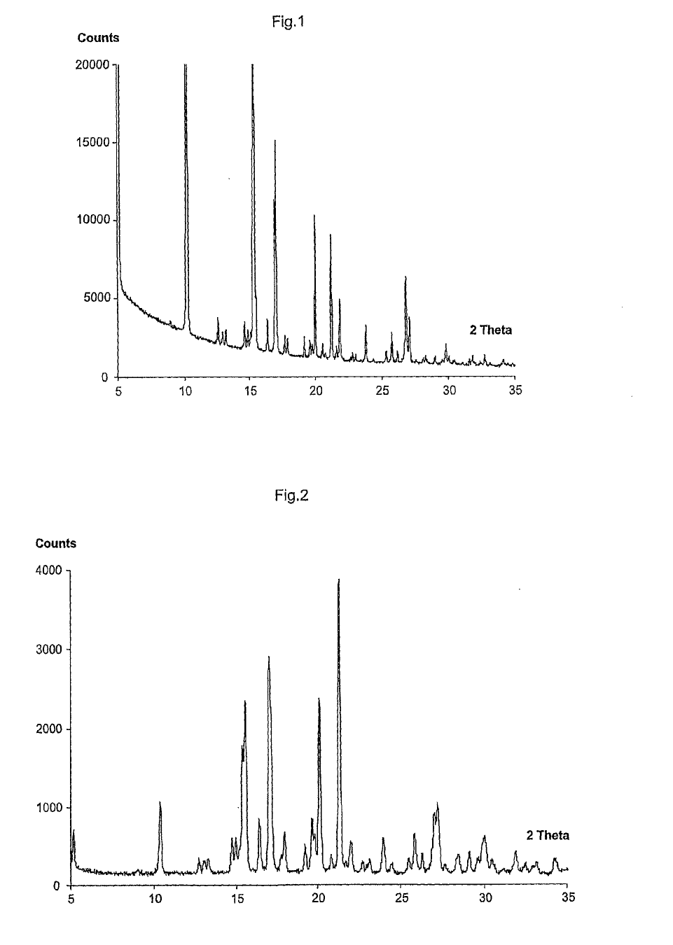

The diffractogram is shown in FIG. 2.

Result from assay: IC50 (μM) 0.01099.

### Example 7

### 2-{2-Chloro-5-{[(2S)-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic Acid

### Method 1

### Step 1: 1-oxa-6-aza-spiro[2.5]octane-6-carboxylic Acid Tert-Butyl Ester

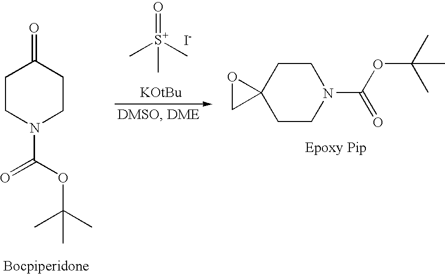

Addition of 4-oxo-piperidine-1-carboxylic acid tert-butyl ester as a DMSO solution to a prepared solution of trimethylsulfoxoniumn iodide and potassium tert-butoxide (Corey-Chaykovsky reagent) in DMSO, provided the epoxy-piperidine.

Potassium tert-butoxide (660 g, 5.89 mol) and DMSO (5.5 L) were charged to a reaction vessel and the mixture cooled to around 20° C. with stirring. Trimethylsulfoxonium iodide (1.24 kg, 5.63 mol) was added in portions over a period of 15-20 min, maintaining the reaction temperature between 20 and 25° C. On completion of the addition, the mixture was maintained at this temperature until a yellow solution was obtained (1-1.5 h). DME (1.5 L) was added to the reaction flask and the solution cooled to 0-5° C. A pre-cooled solution of 4-oxo-piperidine-1-carboxylic acid tert-butyl ester (1 kg, 5.02 mol) in a mixture of DME (1.5 L) and DMSO (500 ml) was transferred into the reaction mixture over a period of around 45 min, maintaining the reaction temperature between 0 and 5° C. On completion of the addition, the reaction mixture was held at this temperature for a further 1-1.5 h. TBME (4 L) was added to the reaction mixture followed by water (6 L) over a period of 30-40 min, maintaining the reaction temperature between 0 and 10° C., then stirring continued for a further 15-20 min at this temperature. The phases were separated and the aqueous layer was extracted with TBME (2×4 L). The combined organic layers were washed with water (2×6 L), dried over sodium sulphate, filtered and the solids washed with TBME (500 ml). The combined filtrates were concentrated under vacuum at below 45° C. to a small volume (1.5 kg). TBME (20 L) was added to the concentrate and solvent was distilled off at below 45° C. to leave a small volume (around 1.3 kg). THF (10 L) was added to the concentrate and solvent was distilled off to leave a solution of 1-oxa-6-aza-spiro[2.5]octane-6-carboxylic acid tert-butyl ester in THF, 1.8 kg, 51.2% w/w, 0.92 kg contained weight, 86% yield.

¹H NMR (399.824 MHz, CDCl₃) δ 3.78-3.65 (m, 2H), 3.43 (ddd, J=13.3, 9.5, 3.7 Hz, 2H), 2.69 (s, 2H), 1.85-1.74 (m, 2H), 1.50-1.40 (m, 11H)

APCI-MS: m/z 114 (MH⁺-(CH₃)₃OCO).

### Step 2: 4-(5-Chloro-2-methoxybenzyl)-4-hydroxypiperidine-1-carboxylic Acid Tert-Butyl Ester

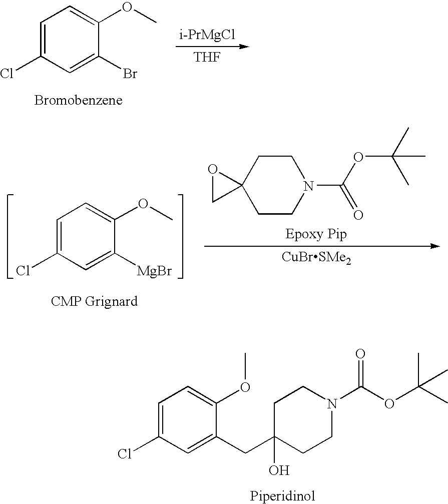

2-Bromo-4-chloroanisole is treated with isopropylmagnesium chloride dissolved in THF to produce the Grignard reagent in situ. A catalytic amount of copper (I) bromide dimethyl sulphide complex (CuBr.SMe₂) and a solution of 1-oxa-6-aza-spiro[2.5]octane-6-carboxylic acid tert-butyl ester in THF are added to produce the desired piperidinol.

A solution of isopropylmagnesium chloride in THF (2 M, 2.96 kg, 3036 ml, 6.07 mol) was added to a stirred solution of 2-bromo-4-chloro-1-methoxybenzene (1.26 kg, 5.69 mol) in THF (5.5 kg) at a temperature of between 15 and 25° C. and stirring continued at this temperature for 6-8 h. Copper(I) bromide dimethylsulphide complex (8.8 g, 42.8 mmol) was added to the reaction mixture and stirring continued at between 17 and 20° C. for 10 min. A solution of 1-oxa-6-aza-spiro[2.5]octane-6-carboxylic acid tert-butyl ester in THF (3.1 kg, 39% w/w, 1.21 kg contained weight, 5.67 mol) was added to the reaction over a period of 20 min, maintaining the temperature between 15 and 20° C., followed by further THF (2.3 kg). After stirring at between 20 and 25° C. for 10-12 h, the reaction mixture was cooled to between 5 and 10° C. and a mixture of water (97 ml) and THF (220 g) added over 20 min followed by ethyl acetate (8 kg) and a solution of ammonium chloride (1.72 kg) in water (9.68 kg). The reaction mixture was warmed to between 25 and 30° C. and stirred at this temperature for around 20 min. The layers were separated, the aqueous layer was extracted with ethyl acetate (8 kg) and the combined organic layers were washed with water 2×6 kg). The organic phase was concentrated under vacuum at 40-45° C. to 2-3 L total volume then heptane (8 kg) added to the solution over a period of 30 min. After cooling to ambient temperature then further cooling to 0-5° C. and holding at this temperature, the solid was collected by filtration, washed with a mixture of ethyl acetate and heptane (1:5, 1.4 kg) followed by heptane (1.5 kg) then dried to afford 4-(5-chloro-2-methoxybenzyl)-4-hydroxypiperidine-1-carboxylic acid tert-butyl ester as a solid, 1.65 kg (82%).

¹H NMR (399.824 MHz, CDCl₃) δ 7.19 (dd, J=8.7, 2.8 Hz, 1H), 7.09 (d, J=2.8 Hz, 1H), 6.82 (d, J=8.7 Hz, 1H), 3.92-3.71 (m, 5H), 3.11 (t, J=11.7 Hz, 2H), 2.80 (br s, 2H), 2.46 (s, exch D₂O, 1H), 1.60-1.42 (m, 11H)

APCI-MS: m/z 256/258 (MH⁺—(CH₃)₃OCO).

### Step 3: 5-Chloro-3H-spiro[1-benzofuran-2,4′-piperidine], Hydrobromic Acid Salt

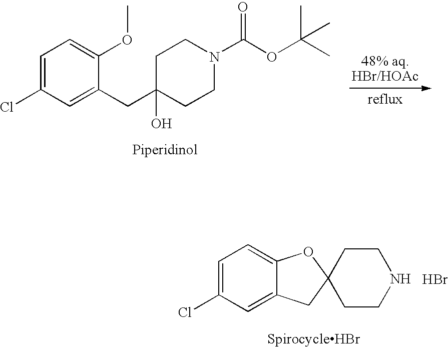

5-Chloro-2-methoxybenzyl)-4-hydroxypiperidine-1-carboxylic acid tert-butyl ester is heated under reflux in a mixture of hydrobromic acid and acetic acid to form the hydrobromic acid salt of the 5-chlorospiropiperidine.

Aqueous hydrobromic acid (48% w/w, 62 ml) was added dropwise to a stirred mixture of 4-(5-chloro-2-methoxybenzyl)-4-hydroxypiperidine-1-carboxylic acid tert-butyl ester (20 g, 56 mmol) and acetic acid (40 ml) over a period of 40 min at a temperature of between 40 and 50° C. Stirring was continued at this temperature for a further 30-40 min on is completion of the addition. The reaction mixture was then heated to reflux for between 6 and 8 h when HPLC analysis showed complete reaction. After cooling to between 20 and 30° C., ethanol (60 ml) was charged to the reaction and stirring continued at between 20 and 25° C. for 20 min. After cooling to between −10 and −15° C. and stirring for 30 min, the solid product was collected by filtration, washed with ethanol (2×20 ml) and dried to afford 5-chloro-3H-spiro[1-benzofuran-2,4′-piperidine], hydrobromic acid salt as an off-white solid, 13.5 g (79%). The combined filtrates were concentrated in vacuo to a volume of 40 ml then ethanol (20 ml) added and the mixture cooled to between −5 and −10° C. The solid product was collected by filtration and washed with ethanol (2×10 ml). After drying, further 5-chloro-3H-spiro[1-benzofuran-2,4′-piperidine], hydrobromic acid salt, 1.4 g (8.2%) was obtained.

¹H NMR (399.826 MHz, D₆-DMSO) δ 8.57 (br s, 2H), 7.28 (m, 1H), 7.15 (dd, J=8.5, 2.3 Hz, 1H), 6.80 (d, J=8.7 Hz, 1H), 3.27-3.08 (m, 4H), 3.12 (s, 2H), 2.06-1.89 (m, 4H).

APCI-MS: m/z 224/226 (MH⁺)

### Step 4: 5-chloro-2-hydroxy-4-methoxybenzoic Acid Methyl Ester

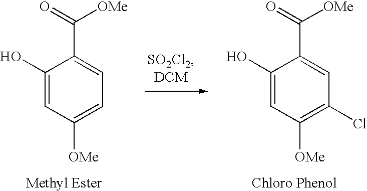

Sulfuryl chloride (274.8 g, 2.0 mol) was charged to a stirred solution of 2-hydroxy-4-methoxybenzoic acid methyl ester (308.2 g, 1.7 mol) in dichloromethane (3.18 L) maintained at between 25 and 30° C. After stirring for 6 h the amount of starting material remaining was 2.3% by HPLC area. Acetic acid (203 g, 3.4 mol) was added to the reaction mixture followed by water (750 ml). The organic phase was separated then solvent distilled off at atmospheric pressure whilst adding methanol so as to maintain roughly constant reaction volume until a head temperature of 60° C. was achieved. A total of 3.5 L methanol was added. The product suspension was cooled to 0 to 5° C., the solid was collected by filtration, washed with methanol (2×200 ml) and dried under vacuum at 50-60° C. The crude solid (342 g) was re-slurried in methanol (3.4 L) then collected by filtration and dried under vacuum at 50-60° C. to afford 5-chloro-2-hydroxy-4-methoxybenzoic acid methyl ester as a solid (316.6 g, 86.5%).

¹H NMR (399.824 MHz, CDCl₃) δ 10.92 (s, 1H), 7.81 (s, 1H), 6.50 (s, 1H), 3.93 (s, 3H), 3.92 (s, 3H)

### Step 5: 5-chloro-2,4-dihydroxybenzoic Acid Methyl Ester

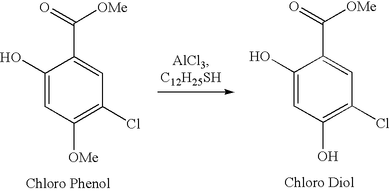

Aluminium chloride (531 g, 4.0 mol) and toluene (3.45 L) were charged to a reaction vessel and stirred. Dodecanethiol (966 g, 4.8 mol) was added over 25 min and the mixture stirred to give a solution then heated to 40 to 50° C. A solution of 5-chloro-2-hydroxy-4-methoxybenzoic acid methyl ester (345.0 g, 1.6 mol) in toluene (3.45 L) was then added over 2 h at 40 to 50° C. The reaction mixture was maintained at this temperature for a further 2 h following the addition when less than 1.0% starting material remained. The reaction was quenched by the slow portionwise addition of water (520 ml) (exothermic) and this was followed by a further water charge (3.45 L), resulting in two clear phases. The organic phase was separated off and filtered at 40 to 50° C. A solvent replacement into heptane was performed under reduced pressure at 55° C. and the product suspension cooled. The solid was collected by filtration, washed with heptane and dried under vacuum to provide 5-chloro-2,4-dihydroxybenzoic acid methyl ester (281.3 g, 87.3%).

¹H NMR (399.826 MHz, D₆-DMSO) δ 11.29 (s, 1H), 10.57 (s, 1H), 7.69 (s, 1H), 6.53 (s, 1H), 3.85 (s, 3H).

### Step 6: 5-Chloro-2-hydroxy-4-(4-methoxybenzyloxy)benzoic Acid Methyl Ester

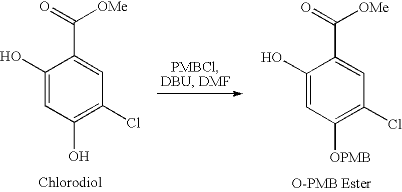

4-Methoxybenzylchloride (37.3 g, 238 mmol) was added to a stirred suspension of 5-chloro-2,4-dihydroxybenzoic acid methyl ester (45.0 g, 222 mmol) and DBU (37.8 g, 248 mmol) in DMF (450 ml) over a period of 3 h at 25° C. with stirring. The reaction was then heated to 65° C. and held for 1 h. After cooling back to 20° C., water (495 ml) was added, the product was collected by filtration, washed with water (2×50 ml) followed by acetonitrile (2×50 ml) then dried under vacuum at 50° C. The crude product (53.5 g, 75%) was suspended in acetonitrile (250 ml), heated to reflux and held for 15 min, cooled to 40° C. then held for 1 h. The solid was collected by filtration, washed with acetonitrile (2×25 ml) then dried under vacuum at 50° C. to provide 5-chloro-2-hydroxy-4-(4-methoxybenzyloxy)benzoic acid methyl ester as a solid 42.9 g (60%).

¹H-NMR (CDCl₃, 300 MHz): δ 10.89 (s, 1H), 7.83 (s, 1H), 7.37 (d, J=8.1 Hz, 2H), 6.93 (d, J=8.1 Hz, 2H), 6.56 (s, 1H), 5.09 (s, 2H), 3.92 (s, 3H), 3.82 (s, 3H)

APCI-MS (-ve): m/z 321 [M(—H)]⁻

### Step 7: 5-Chloro-2-hydroxy-4-(4-methoxybenzyloxy)-N-methylbenzamide

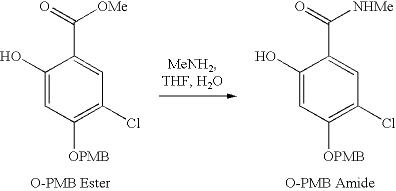

An aqueous solution of methylamine (40% w/w, 500 ml) was added to a stirred suspension of 5-chloro-2-hydroxy-4-(4-methoxybenzyloxy)benzoic acid methyl ester (100 g, 0.31 moles) in THF (500 ml). The mixture was heated to 50-56° C. and the resulting clear solution held at this temperature for 4 h, then cooled to ambient temperature and stirred overnight. Solvent was distilled off under reduced pressure until 600 ml had been removed, maintaining a roughly constant reaction volume by the dropwise addition of water (600 ml). The temperature of the reaction mixture increased from 22° C. to 47° C. during the course of the distillation. The resulting suspension was cooled to 5° C. and stirred for 30 min. The product was collected by filtration and dried under vacuum at 50° C. to leave 5-chloro-2-hydroxy-4-(4-methoxybenzyloxy)-N-methylbenzamide as a solid (94.6 g, 95% yield).

¹H NMR (399.826 MHz, D₆-DMSO) δ 8.93 (br s, 1H), 7.93 (s, 1H), 7.39 (d, J=9.5 Hz, 2H), 6.96 (d, J=9.5 Hz, 2H), 6.69 (s, 1H), 5.11 (s, 2H), 3.76 (s, 3H), 2.78 (s, 3H)

APCI-MS: m/z 322/324 (MH)⁺

### Step 8: 5-Chloro-4-(4-methoxybenzyloxy)-N-ethyl-2-((S)-1-oxiranylmethoxy)benzamide

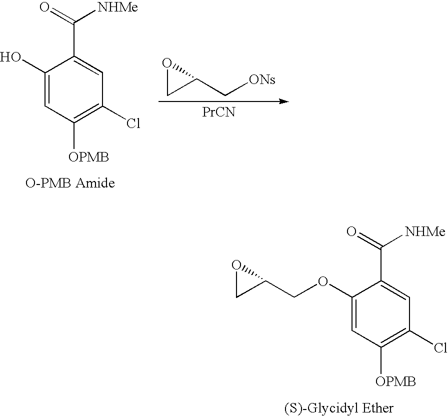

A solution of 3-nitrobenzenesulfonic acid (S)-1-oxiranylmethyl ester in butyronitrile (0.317 kg of a 28.2% w/w solution, 89.4 g contained weight, 345 mmol, 1.1 eq) was diluted with butyronitrile (0.238 kg) and cooled to 7° C. with stirring. 5-Chloro-2-hydroxy-4-(4-methoxybenzyloxy)-N-methylbenzamide (100 g, 0.311 mmol, 1.0 eq) was added followed by cesium carbonate (25.3 g, 77.7 mmol) and the mixture heated to 55° C. Two further portions of cesium carbonate (25.3 g each, 77.7 mmol) were added to the reaction mixture after holding at 55° C. for 30 min and cooling the reaction mixture back to 7° C. prior to each addition. After 1 h 40 min further cesium carbonate (25.3 g, 77.7 mmol) was added to the reaction mixture and after an additional 1 h, a final portion of cesium carbonate (50.7 g 156 mmol) was added to the reaction mixture at 55° C. On completion of the reaction, water (1 kg) added and the reaction mixture cooled to 7° C. After stirring for 1 h, the solid product was collected by filtration, washed with water (150 ml) and methanol (100 ml) then dried under vacuum at 45° C. to leave 5-chloro-4-(4-methoxybenzyloxy)-N-methyl-2-((S)-1-oxiranylmethoxy)benzamide as a white solid, 93.6 g (79.7%).

¹H NMR (399.826 MHz, D₆-DMSO) δ 8.01-7.93 (m, 1H), 7.78 (s, 1H), 7.42 (d, J=9.1 Hz, 2H), 7.03 (s, 1H), 6.98 (d, J=9.1 Hz, 2H), 5.20 (s, 2H), 4.55 (dd, J=11.5, 2.6 Hz, 1H), 4.12 (dd, J=11.7, 6.0 Hz, 1H), 3.76 (s, 3H), 3.49-3.44 (m, 1H), 2.90 (t, J=4.6 Hz, 1H), 2.81 (d, J=4.6 Hz, 3H), 2.78-2.74 (m, 1H).

APCI-MS: m/z 378/380 (MH⁺)

### Step 9: 5-Chloro-2-{([(2S)-3-(5-chloro-3H-spiro[1-benzofuran-2,4′-piperidin]-1′-yl)-2-hydroxypropyl]oxy}-4-hydroxy-N-methylbenzamide, Trifluoroacetic Acid Salt

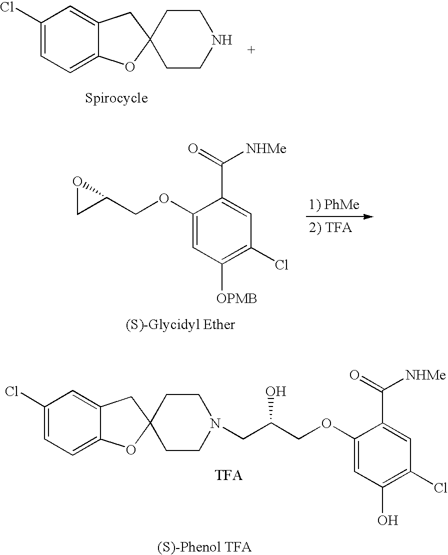

A suspension of 5-chloro-3H-spiro[1-benzofuran-2,4′-piperidine], hydrobromic acid salt (Step 3; 42.85 g, 141 mmol) in toluene (440 ml) was stirred with aqueous ammonium hydroxide solution (28% w/w, 55 ml) for 30 min. The mixture was then filtered to remove a small amount of a solid and the layers allowed to separate. The aqueous phase was extracted with toluene (220 ml) and combined with the organic phase from the first separation to leave a solution of 5-chlorospiro[3H-benzofuran-2,4′-piperidine] in toluene. To this was added 5-chloro-4-(4-methoxybenzyloxy)-N-methyl-2-((S)-1-oxiranylmethoxy)benzamide (Step 8; 50 g, 132 mmol) and the mixture heated at 80° C. for 22 h. The turbid solution was filtered at 80° C. then cooled to ambient temperature to leave 5-chloro-2-{[(2S)-3-(5-chloro-3H-spiro[1-benzofuran-2,4′-piperidin]-1′-yl)-2-hydroxypropyl]oxy}-4-(p-methoxybenzyloxy)-N-methylbenzamide as a suspension in toluene.

To this suspension was added trifluoroacetic acid (220 g, 1.93 mol) at a temperature of between 20 and 25° C. with stirring. After stirring for 3 h at this temperature, the mixture was concentrated by distillation under vacuum until a residue of ca 200 ml remained. Isopropanol (150 ml) was added and solvent distilled off until the volume of the residue was ca 200 ml. This operation was repeated one more time. Methanol (200 ml) was added and solvent distilled off at atmospheric pressure until 200 ml of distillate had been removed. The residue was dissolved in methanol (400 ml) and stirred overnight. Some sticky solid was removed by filtration and the filtrate was distilled at atmospheric pressure, replacing the solvent removed with isopropanol (300 ml). The suspension was cooled in an ice-water bath then the solid product was collected by filtration, washed with isopropanol (2×50 ml) then dried in a vacuum oven at 50° C. to leave a 5-chloro-2-{[(2S)-3-(5-chloro-3H-spiro[1-benzofuran-2,4′-piperidin]-1′-yl)-2-hydroxypropyl]oxy}-4-hydroxy-N-methylbenzamide, trifluoroacetic acid salt as an off-white powder, 66.1 g (84% over 2 stages).

¹H-NMR (D₆-DMSO, 400 MHz): δ 8.05 (d, J=4.6, NH), 7.73 (s, 1H), 7.30 (m, 1H), 7.18-7.14 (m, 1H), 6.82-6.78 (m, 1H), 6.73 (s, 1H), 4.42 (m, 1H), 4.05 (s, 2H), 3.57 (m, 2H), 3.45-3.40 (m, 1H), 3.27-3.11 (m, 5H), 2.81 (d, J=4.8, 3H), 2.18-2.08 (m, 4H); APCI-MS: m/z 481 (MH⁺).

APCI-MS: m/z 481/483/485 (MH⁺).

Spectral data on an isolated sample of the intermediate PMB-protected compound:

¹H NMR (399.826 MHz, D₆-DMSO) δ 8.33-8.27 (m, 1H), 7.83 (s, 1H), 7.43 (dd, J=6.7, 2.1 Hz, 2H), 7.25-7.22 (m, 1H), 7.10 (dd, J=8.6, 2.4 Hz, 1H), 7.02 (s, 1H), 6.98 (d, J=6.7 Hz, 2H), 6.74 (d, J=8.5 Hz, 1H), 5.27 (s, exch D₂O, 1H), 5.23 (s, 2H), 4.29-4.22 (m, 1H), 4.11-4.02 (m, 2H), 3.76 (s, 3H), 3.00 (s, 2H), 2.80 (m, 3H), 2.70-2.56 (m, 2H), 1.88-1.70 (m, 4H). Remaining signals were coincident with DMSO at 2.5 ppm.

APCI-MS: m/z 601/603/605 (MH⁺)

### Step 10: 2-{2-Chloro-5-{[(2S)-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic Acid

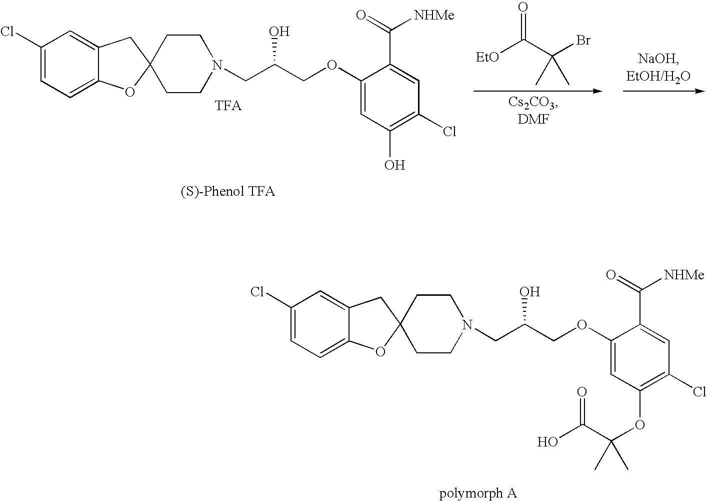

### Method 1

5-Chloro-2-{[(2S)-3-(5-chloro-3H-spiro[1-benzofuran-2,4′-piperidin]-1′-yl)-2-hydroxypropyl]oxy}-4-hydroxy-N-methylbenzamide TFA (135.5 g) was placed in a 2 L jacketed vessel and treated sequentially with caesium carbonate (3.0 eq), ethyl-2-bromoisobutyrate (3.0 eq) and then DMF (675 ml). The mixture was heated to 60° C. and stirred overnight at this temperature. The mixture was cooled to 20° C., treated with water (1.0 L) and then extracted with ethyl acetate (1×600 ml and 1×400 ml). The ethyl acetate extracts were combined and evaporated to dryness to give an orange oil (221.07 g). The residue was redissolved in ethanol (675 ml) and treated with a solution of sodium hydroxide (27.2 g in 270 ml water) with stirring. After 30 min the solvent was evaporated and the residue was treated with ammonium acetate (140 g) in water (1.35 L). The resulting slurry was stirred overnight and then filtered. The filter cake was slurry washed with water (1×135 ml and 1×540 ml), ethanol (270 ml), TBME (135 ml), treated with ethanol (1 L) at 60° C. for 18 h and then filtered. The filter cake was washed with ethanol (135 ml). The solid was dried overnight in a vacuum oven at 50° C. to give the titled zwitterion as polymorph A (102.3 g; 80% over 2 steps)

¹H-NMR (D₆-DMSO, 400 MHz): δ 13.41 (br s, 1H), 9.60-9.35 (m, 1H), 8.13 (d, J=4.6 Hz, 1H), 7.75 (s, 1H), 7.30 (s, 1H), 7.16 (d, J=8.7 Hz, 1H), 6.80 (d, J=8.5 Hz, 1H), 6.19 (s, 1H), 4.40 (br.s, 1H), 4.00 (d, J=4.4 Hz, 2H), 3.62-3.15 ((m, 6H), 3.11 (s, 2H), 2.82 (d, J=4.7 Hz, 3H), 2.50 (m, 4H), 1.60 (s, 6H);

APCI-MS: m/z 567 (MH⁺).

Spectral data for an isolated sample of the intermediate ester:

¹H NMR (399.826 MHz, D₆-DMSO) δ 8.27 (m, 1H), 7.85 (s, 1H), 7.23 (m, 1H), 7.10 (dd, J=8.5, 2.3 Hz, 1H), 6.74 (d, J=8.5 Hz, 1H), 6.54 (s, 1H), 5.26 (m, exch D₂₀, 1H), 4.23 (q, J=7.1 Hz, 2H), 4.16-4.02 (m, 3H), 3.92 (dd, J=9.2, 6.2 Hz, 1H), 3.00 (s, 2H), 2.80 (d, J=4.9 Hz, 3H), 1.87-1.68 (m, 4H), 1.61 (s, 6H), 1.21 (t, J=14.9 Hz, 3H). Remaining signals partially overlapping DMSO signal.

APCI-MS: m/z 595/597/599 (MH⁺)

### Method 2

A solution of 5-chloro-2-{[(2S)-3-(5-chloro-3H-spiro[1-benzofuran-2,4′-piperidin]-1′-yl)-2-hydroxypropyl]oxy}-4-hydroxy-N-methylbenzamide, trifluoroacetic acid salt (25.0 g, 42.0 mmol) in NMP (67 ml) was added over a period of 45 min to a stirred suspension of caesium carbonate (41.0 g, 126 mmol) in NMP (67 ml), maintaining the temperature of the mixture below 30° C., followed by an NMP line rinse (4 ml). Ethyl 2-bromoisobutyrate (24.6 g, 126 mmol) was then added to the reaction mixture over a period of 45 min followed by an NMP line rinse (4 ml). The reaction mixture was heated to 70° C. and stirred at this temperature for 11.5 h. After cooling to ambient temperature, the mixture was diluted with TBME (50 ml) then water (175 ml) was added over a period of around 1 h (exothermic addition). Further TBME (105 ml) was charged and the mixture stirred for around 30 min then the layers were allowed to separate. The aqueous layer was extracted with TBME (2×70 ml) and the combined organic layers were concentrated to a volume of approximately 90 ml. Ethanol (110 ml) was added and the volume reduced to 90 ml by evaporation. A further ethanol charge (110 ml) was added and the volume reduced again to 90 ml by evaporation to afford 2-{2-chloro-5-{[(2S)-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic acid ethyl ester as a solution in ethanol, total weight 81.31 g.

A solution of 2-{2-chloro-5-{[(2S)-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic acid ethyl ester in ethanol (952 g total weight, contained weight 366.2 g, 614 mmol) was diluted with ethanol (1.09 L) and warmed to 44° C. with stirring. To this was added a solution of sodium hydroxide (73.8 g, 1.85 mol) in water (732 ml) over a period of 30 min. After holding at 40-45° C. for 2.5 h, the solution was decanted away from the polymeric by product and filtered. A solution of citric acid (101 g) in water (1.46 L) was added to the filtrate over a period of 1 h 50 min. The solid was collected by filtration, washed with water (1.5 L), ethanol (1.5 L then 375 ml) and dried in a vacuum oven at 65° C. to yield crude 2-{2-chloro-5-{[(2S-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic acid as a pale yellow solid, weight 301.63 g (86%).

A slurry of crude 2-{2-chloro-5-{[(2S)-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic acid (9.0 g) in NMP (54 ml) was heated to 80° C. with stirring to dissolve the solid then cooled to around 65° C. Ethanol (333 ml) was charged over a period of 35 min, maintaining the reaction temperature between 60 and 70° C., which caused crystallization of the product. After a further 30 min at this temperature the slurry was cooled to between 10 and 15° C. over 1 hr, then held at this temperature for around 30 min. The solid was collected by filtration, washed with ethanol (45 ml), pulled dry on the filter then dried in a vacuum oven at 60° C. 2-{2-Chloro-5-{[(2S)-3-(5-chloro-3H-spiro[1-benzofuran-2,41-piperidin]-1′-yl)-2-hydroxypropyl]oxy}-4-[(methylamino)carbonyl]phenoxy}-2-methylpropanoic acid was obtained as a white solid, weight 5.49 g (61%).

The resulting solid (5 g) was slurried in NMP (50 ml) and heated to 60° C. and held at between 60 and 65° C. for 30 min with stirring. Water (50 ml) was charged to the resulting solution over a period of 35 min, maintaining the temperature between 60 and 65° C., which caused crystallization of the product. After a further 30 min at this temperature the slurry was cooled to ambient temperature then held at this temperature for 30 min. The mixture was further cooled to between 0 and 4° C. and held for 30 min. The solid was collected by filtration, washed with water (25 ml), ethanol (25 ml), pulled dry on the filter then dried in a vacuum oven at 60° C. 2-{2-Chloro-5-{[(2S)-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic acid was obtained as a white solid (polymorph A), weight 4.82 g (96%).

The title compound exhibits at least the following characteristic X-ray powder diffraction (XRPD) peaks (expressed in degrees 2θ) (the margin of error being consistent with the United States Pharmacopeia general chapter on X-ray diffraction (USP941)—see the United States Pharmacopeia Convention. X-Ray Diffraction, General Test <941>. United States Pharmacopeia, 25th ed. Rockville, Md.: United States Pharmacopeial Convention; 2002:2088-2089):

(1) 5.1, 10.2 and 12.9, or
(2) 5.1, 8.9 and 13.2, or
(3) 8.9, 10.2, 12.9, 15.1, 17.0 and 21.2 or
(4) 5.1, 8.9, 10.2, 14.6, 15.4, 21.2 and 25.8 or
(5) 5.1, 8.9, 10.2, 12.6, 14.6, 15.1 and 17.0 or
(6) 5.1, 10.2, 12.6, 13.2, 14.6, 15.1, 17.0, 17.9, 21.2 and 21.8 or
(7) 5.1, 8.9, 10.2, 12.6, 13.2, 14.6, 14.9, 16.4, 19.2, 21.8 and 27.1 or
(8) 5.1, 8.9, 10.2, 12.6, 12.9, 13.2, 14.6, 14.9, 15.1, 15.4, 16.4, 17.9, 19.2, 20.0, 21.8 and 25.8

The diffractogram is shown in FIG. 1.

### Example 8

### Form B, S-Enantiomer

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form B

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form A (300 mg) was dissolved in chloroform (200 ml) by stirring at 30° C. for 3 h. The solvent was evaporated to air at 20° C. to give a solid white, moderately crystalline 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form B.

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form B exhibits at least the following characteristic X-ray powder diffraction (XRPD) peaks (expressed in degrees 2θ) (the margin of error being consistent with the United States Pharmacopeia general chapter on X-ray diffraction (USP941)—see the United States Pharmacopeia Convention. X-Ray Diffraction, General Test <941>. United States Pharmacopeia, 25th ed. Rockville, Md.: United States Pharmacopeial Convention; 2002:2088-2089):

5.6, 7.6, 8.6, 13.1, 17.0, 18.4.

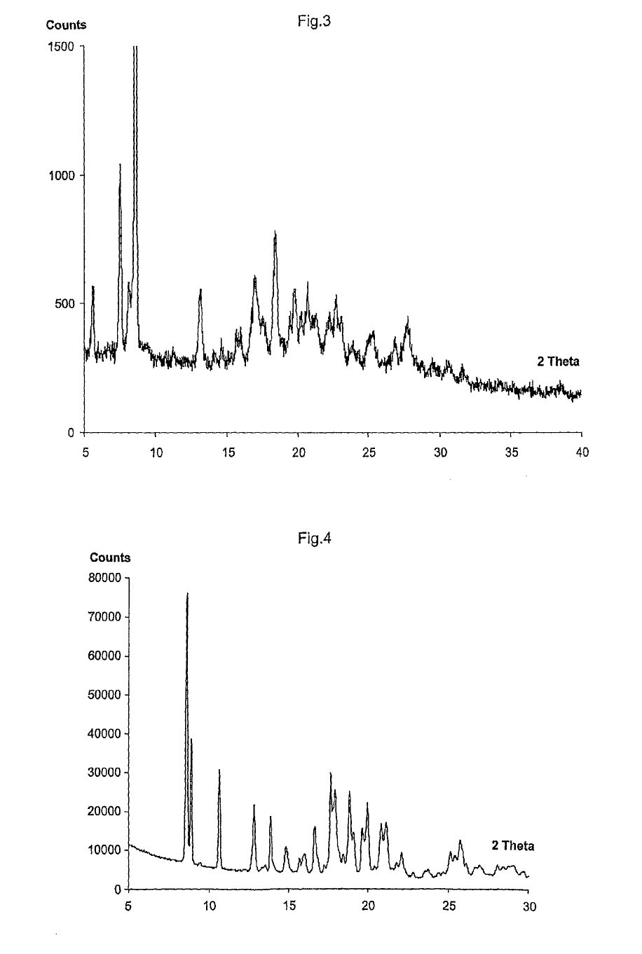

The diffractogram is shown in FIG. 3.

### Example 9

### Form C, S-Enantiomer

### 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic Acid Form C

### Method A:

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form A (micronized, 310 mg) was dissolved in THF (dried; 200 ml) and stirred at 30° C. for 24 h. A white milky suspension was produced. The material was allowed to sediment at room temperature for 24 h. The supernatant was removed and the sedimented material dried under vacuum (oil pump) at 80° C. for 24 h to remove remaining THF, yielding 2-{2 Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form C.

### Method B:

Equal amounts of 2-{2-Chloro-5-{[(2S)-3-(5-chloro-1′H,3H-spiro[1-benzofuran-2,4′-piperidin]-1-yl)-2-hydroxypropyl]oxy}-4-[(methylamino)carbonyl]phenoxy}-2-methylpropanoic acid Form A, Form C, and Form D (1 mg of each) were suspended in dichloromethane (0.65 ml). The mixture was shaken at 35° C. for 2 days, yielding 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form C.

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form C exhibits at least the following characteristic X-ray powder diffraction (XRPD) peaks (expressed in degrees 2θ) (the margin of error being consistent with the United States Pharmacopeia general chapter on X-ray diffraction (USP941)—see the United States Pharmacopeia Convention. X-Ray Diffraction, General Test <941>. United States Pharmacopeia, 25th ed. Rockville, Md.: United States Pharmacopeial Convention; 2002:2088-2089):

(1) 4.5, 8.9 and 12.8, or
(2) 4.5, 8.6 and 10.6, or
(3) 4.5, 8.9, 10.6, 12.8, 14.8 and 17.6 or
(4) 8.6, 8.9, 12.8, 13.9, 15.7, 16.6 and 18.8 or
(5) 4.5, 8.6, 8.9, 10.6, 13.9, 15.7, 16.0, 16.6 and 17.9 or
(6) 4.5, 8.9, 10.6, 12.8, 13.9, 14.8, 15.7, 17.6, 18.8 and 20.0 or
(7) 4.5, 8.6, 8.9, 10.6, 12.8, 13.9, 15.7, 16.0, 16.6, 17.9, 18.8, 20.0, 20.9 and 21.2.

The diffractogram is shown in FIG. 4.

### Example 10

### Form D, S-Enantiomer

### 2-{2-Chloro-5-{[(2S)-3-(5-chloro-1′H,3H-spiro[1-benzofuran-2,4L-piperidin]-1′-yl)-2-hydroxypropyl]oxy}-4-[(methylamino)carbonyl]phenoxy}-2-methylpropanoic Acid Form D

Heating of 2-{2-Chloro-5-{[(2)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form B to 140° C. under N₂ atmosphere yields 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form D.

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form D exhibits at least the following characteristic X-ray powder diffraction (XRPD) peaks (expressed in degrees 2θ) (the margin of error being consistent with the United States Pharmacopeia general chapter on X-ray diffraction (USP941)—see the United States Pharmacopeia Convention. X-Ray Diffraction, General Test <941>. United States Pharmacopeia, 25th ed. Rockville, Md.: United States Pharmacopeial Convention; 2002:2088-2089):

5.4, 9.8, 12.3, 13.6, 16.9, 19.2, 19.5 and 21.3.

The diffractogram is shown in FIG. 5.

### Example 11

### Form F, S-Enantiomer

### 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic Acid Form F

### Method A

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form A, Form C and Form D (37, 71 and 41 mg respectively) were suspended in methanol (4.0 ml). The slurry was stirred at 35° C. for 4 days. The solid material was isolated by centrifugation (8000 rpm, 30 min, 22° C.) and dried under vacuum for 18 h, yielding 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form F.

### Method B

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic Acid Form A (658 mg) was suspended in methanol (20 ml). The suspension was heated to 60° C. with stirring for 18 h. The temperature was adjusted to 35° C., thereafter 5 mg -{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form F was added for seeding. The suspension was left at 35° C. with stirring for 72 h. The solid material was isolated by centrifugation and dried under vacuum at 40° C. for 24 h, yielding 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form F

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form F exhibits at least the following characteristic X-ray powder diffraction (XRPD) peaks (expressed in degrees 2θ) (the margin of error being consistent with the United States Pharmacopeia general chapter on X-ray diffraction (USP941)—see the United States Pharmacopeia Convention. X-Ray Diffraction, General Test <941>. United States Pharmacopeia, 25th ed. Rockville, Md.: United States Pharmacopeial Convention; 2002:2088-2089):

(1) 7.5, 9.2 and 10.7, or
(2) 7.5, 8.9 and 11.1, or
(3) 7.5, 8.9, 9.2, 11.1, 12.2 and 16.3 or
(4) 8.9, 9.2, 10.7, 11.1, 11.7, 12.2 and 15.1 or
(5) 7.5, 8.9, 9.2, 10.7, 11.7, 12.2, 13.8, 15.1, 16.7 and 18.5 or
(6) 7.5, 8.9, 9.2, 11.1, 11.9, 13.8, 15.1, 16.3, 17.8, 18.3, 18.7 and 20.9 or
(7) 7.5, 8.9, 9.2, 10.7, 11.1, 11.7, 12.2, 13.8, 15.1, 18.3, 18.7, 19.7, 21.4, 22.3 and 24.0 or
(8) 7.5, 9.2, 10.7, 11.7, 11.9, 12.2, 13.8, 15.1, 16.3, 16.7, 17.8, 18.3, 19.2, 19.7, 20.9, 21.4 and 22.3.

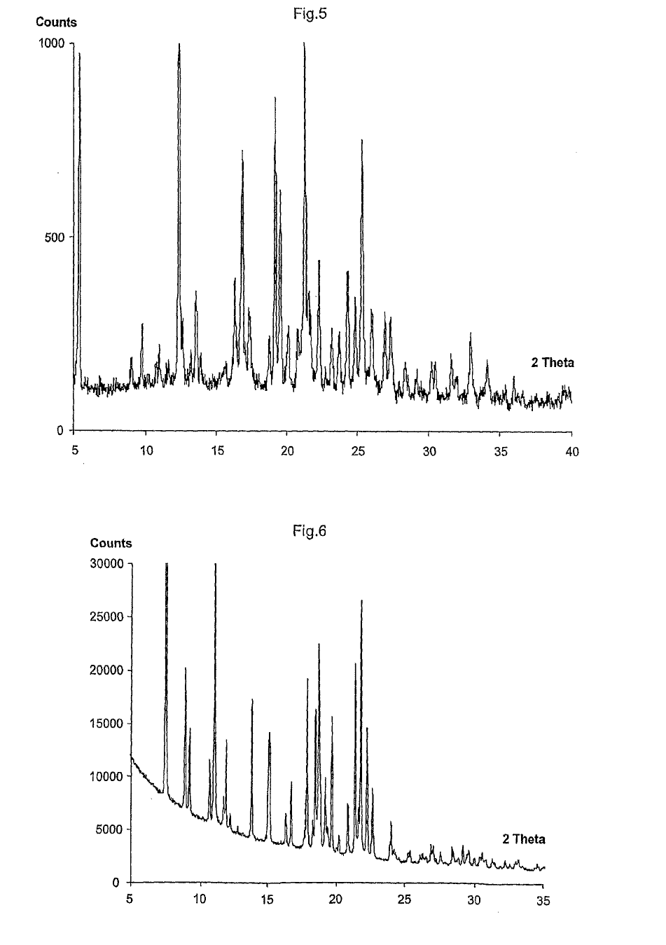

The diffractogram is shown in FIG. 6.

### Example 12

### Form G, S-Enantiomer

### 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic Acid Form G

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form A is dried under flow of dry N2 for 1 h yielding 2-{2-Chloro-5-{[(2R)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form G.

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form G exhibits at least the following characteristic X-ray powder diffraction (XRPD) peaks (expressed in degrees 2θ) (the margin of error being consistent with the United States Pharmacopeia general chapter on X-ray diffraction (USP941)—see the United States Pharmacopeia Convention. X-Ray Diffraction, General Test <941>. United States Pharmacopeia, 25th ed. Rockville, Md.: United States Pharmacopeial Convention; 2002:2088-2089):

(1) 4.8, 12.2 and 15.4, or
(2) 4.8, 9.7 and 13.7, or
(3) 9.7, 13.7, 14.5, 15.6, 17.1 and 20.3 or
(4) 4.8, 13.7, 14.5, 15.4, 16.3, 17.1 and 20.3 or
(5) 4.8, 9.7, 13.7, 14.5, 15.6, 16.3 and 19.7 or
(6) 9.7, 12.2, 13.7, 14.5, 15.6, 16.3, 19.4, 20.3, 21.4 and 23.1 or
(7) 9.7, 13.7, 14.5, 15.6, 16.3, 19.7, 20.3, 20.8, 21.4, 23.1 and 25.5 or
(8) 4.8, 9.7, 12.2, 13.7, 15.4, 16.3, 17.1, 19.4, 19.7, 20.3, 20.8, 21.4, 23.1 and 25.5.

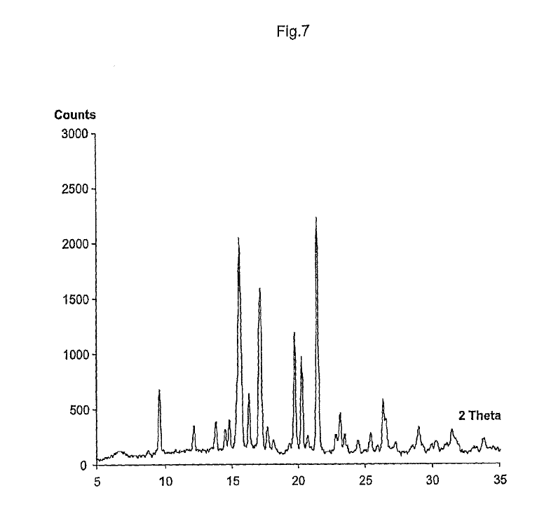

The diffractogram is shown in FIG. 7.

### Example 13

### 2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methylpropanoic Acid Hydrochloride

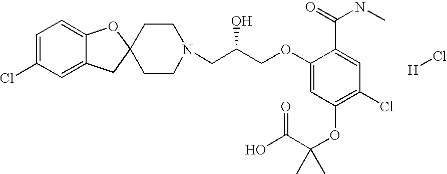

2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methylpropanoic acid (264 mg, 0.5 mmol) was dissolved in a mixture of 1 M hydrochloric acid (1.0 ml) in acetonitrile (1 ml). Water (2 ml) was added giving a sticky precipitate. More acetonitrile was added until a solution was obtained. The solution was diluted with water (2 ml) and set aside in the hood for slow evaporation of acetonitrile. The titled compound precipitated as a white solid (241 mg, 80%).

¹H NMR (299.945 MHz, cd3od) δ 7.81 (s, 1H), 7.22-7.20 (m, 1H), 7.11 (dd, J=8.6, 2.3 Hz, 1H), 6.76 (s, 1H), 6.75 (d, J=8.5 Hz, 1H), 4.55-4.46 (m, 1H), 4.11 (dd, J=7.5, 4.6 Hz, 2H), 3.75-3.35 (m, 6H), 3.16 (s, 2H), 2.94 (s, 3H), 2.34-2.13 (m, 4H), 1.66 (s, 6H)

APCI-MS m/z 567/569 (MH⁺)

Chloride analysis: molratio base/chloride 1/1

The title compound exhibits at least the following characteristic X-ray powder diffraction (XRPD) peaks (expressed in degrees 2θ) (the margin of error being consistent with the United States Pharmacopeia general chapter on X-ray diffraction (USP941)—see the United States Pharmacopeia Convention. X-Ray Diffraction, General Test <941>. United States Pharmacopeia, 25th ed. Rockville, Md.: United States Pharmacopeial Convention; 2002:2088-2089):

(1) 7.6, 7.9, 20.6, 21.3, 22.9 and 23.8 or
(2) 9.7, 13.7, 14.5, 16.2, 16.4, 19.6, 20.6, 21.3, 22.4, 22.9 and 23.8 or
(3) 5.5, 7.6, 7.9, 13.4, 14.5, 15.2, 15.9, 16.2, 16.4, 19.6, 20.6, 21.3, 22.4, 22.9 and 23.8.

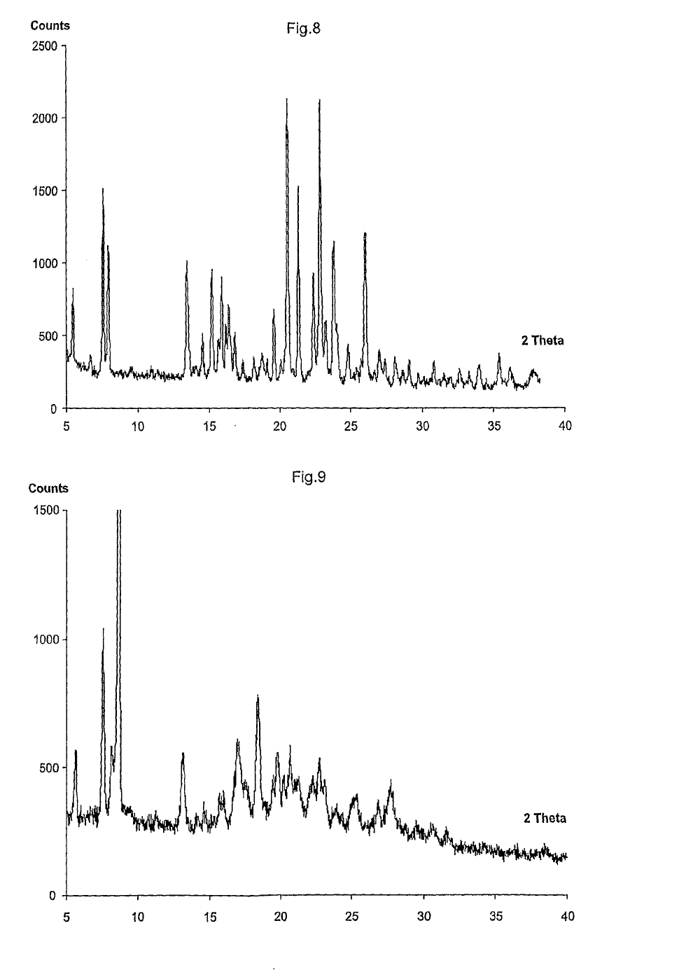

The diffractogram is shown in FIG. 8.

### Example 14

### 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic Acid Sodium Hydroxide

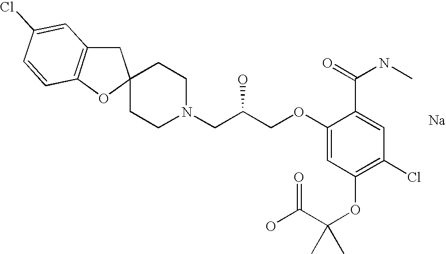

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid (770 mg) is dissolved in EtOH (680 ml) at 70° C. NaOH (40 mg) is dissolved in water (5 ml). The aq. NaOH solution (1.7 ml) is added to the 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid solution (170 ml). The precipitate is collected by filtration.

APCI-MS: m/z 567 (MH⁺).

2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid Form G exhibits at least the following characteristic X-ray powder diffraction (XRPD) peaks (expressed in degrees 2θ) (the margin of error being consistent with the United States Pharmacopeia general chapter on X-ray diffraction (USP941)—see the United States Pharmacopeia Convention. X-Ray Diffraction, General Test <941>. United States Pharmacopeia, 25th ed. Rockville, Md.: United States Pharmacopeial Convention; 2002:2088-2089):

(1) 7.6, 8.6 and 18.4 or
(2) 5.6, 7.6, 8.6, 13.1, 17.0 and 18.4.

The diffractogram is shown in FIG. 9.

### Example 15

### 2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoic Acid Trifluoracetate

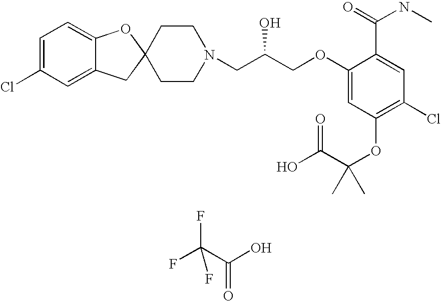

Prepared according to the process described in example 15 from 2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)-carbonyl]phenoxy}-2-methylpropanoic acid (264 mg, 0.5 mmol) and TFA (74 μL, 1.0 mmol) in acetonitrile (3 ml) and water (3 ml) and freezedrying the solution.

The titled compound was obtained as a white solid (328 mg, 96%).

APCI-MS m/z 567/569 (MH⁺)

### Example 16

### 2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoic acid p-toluensulfonate

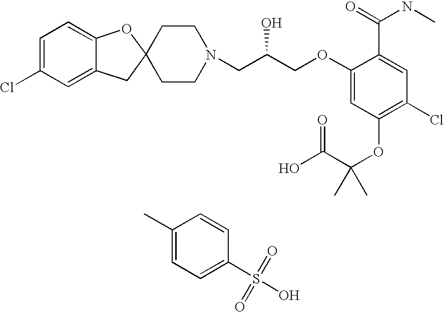

2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methylpropanoic acid (264 mg, 0.5 mmol) and p-toluensulfonic acid monohydrate (105 mg, 0.55 mmol) was dissolved in a mixture of acetonitrile (1 ml) and water (1 ml). No solid precipitate was obtained after slow evaporation. The oily precipitate was vacuum-dried to give the titled compound as a white solid (319 mg, 86%).

¹H NMR (299.945 MHz, cd3od) δ 7.81 (s, 1H), 7.69 (d, J=7.6 Hz, 2H), 7.21 (d, J=7.7 Hz, 2H), 7.19 (s, 1H), 7.11 (d, J=9.1 Hz, 1H), 6.74 (d, J=8.4 Hz, 2H), 6.75 (s, 1H), 4.46-4.57 (m, 1H), 4.02-4.16 (m, 2H), 3.59-3.79 (m, 2H), 3.33-3.56 (m, 4H), 3.12 (s, 2H), 2.92 (s, 3H), 2.35 (s, 3H), 2.12-2.28 (m, 4H), 1.66 (s, 6H)

APCI-MS m/z 567/569 (MH⁺)

### Example 17

### 2-{2-Chloro-5-{[(2S)-3-(5-chloro-1′H,3H-spiro[1-benzofuran-2,4′-piperidin]-1′-yl)-2-hydroxypropyl]oxy}-4-[methylamino)carbonyl]phenoxy}-2-methylpropanoic Acid

### Step 1. 4-(1-tert-butoxycarbonyl-1-methylethoxy)-5-chloro-2-hydroxybenzoic Acid Methyl Ester

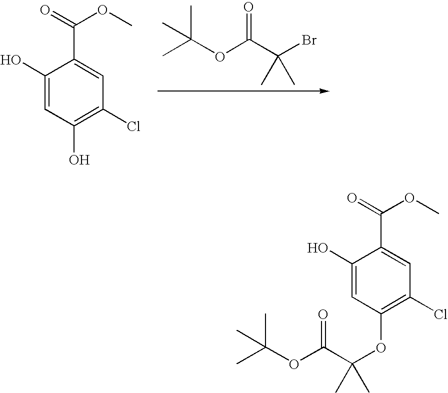

To a solution of 5-chloro-2,4-dihydroxybenzoic acid methyl ester (10.2 g, 10.0 g at 100% w/w, 0.0493 mol, 1.0 mol eq) in N-methylpyrrolidone (40 ml, 4.0 rel vol), was added potassium carbonate (17.40 g, 17.05 g at 100% w/w, 0.1233 mol, 2.5 mol eq) with stirring. 2-Bromo-2-methyl-propionic acid tert-butyl ester (67.42 g, 66.07 g at 100% w/w, 0.2961 mol, 6.0 mol eq) was added in one portion together followed by tetrabutylammonium bromide (3.25 g, 3.18 g at 100% w/w, 0.0098 moles, 0.2 mol eq). The temperature of the reaction mass was raised to 60-65° C. and maintained at this temperature for 16 h. On completion, the reaction mixture was cooled 30-35° C. The insoluble potassium salts were removed by filtration through Celite and the solids were washed with N-methyl pyrrolidone (20 ml, 2.0 rel vol). The pH of the combined filtrates was adjusted to around 4 using dilute HCl solution then water (100 ml, 10.0 rel vol) added. The solution was extracted with dichloromethane (100 ml, 10 rel vol), the organic layer was washed with water (150 ml, 15.0 rel vol) then evaporated to dryness at 35° C. under vacuum. The excess of 2-bromo-2-methyl-propionic acid tert-butyl ester and 2-methylacrylic acid tert-butyl ester by product were removed by applying a high vacuum (20-25 mbar) at 60-65° C. for approximately one h. 4-(1-tert-Butoxycarbonyl-1-methylethoxy)-5-chloro-2-hydroxybenzoic acid methyl ester was obtained as an oil, weight 16.0 g (72.2% yield).

¹H NMR (300 MHz, CDCl₃): δ 10.73 (s, 1H), 7.82 (s, 1H), 6.36 (s, 1H), 3.92 (s, 3H), 1.66 (s, 6H), 1.44 (s, 9H).

### Step 2. 2-(2-Chloro-5-hydroxy-4-methylcarbamoylphenoxy)-2-methylpropionic Acid Tert-Butyl Ester

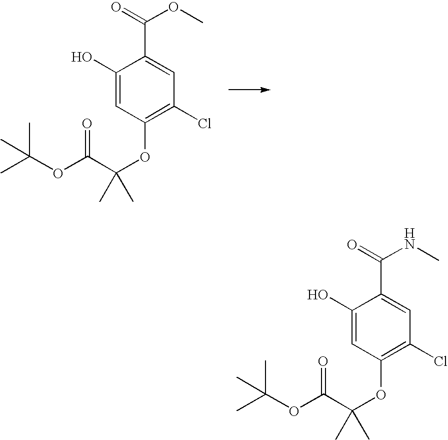

To an aqueous solution of methylamine (40% w/w, 160 ml, 12.6 rel vol) was added 4-(1-tert-butoxycarbonyl-1-methylethoxy)-5-chloro-2-hydroxybenzoic acid methyl ester (16.0 g, 12.27 g at 100%, 0.035 mol, 1.0 mol eq) and the mixture stirred for 1-2 h at 25 to 30° C. After completion of the reaction, the reaction mixture was filtered through a Celite bed to separate some insoluble material. The Celite bed was washed with water (32 ml, 2.60 rel vol) and the combined filtrates de-gassed under vacuum (150 mbar) at 30-35° C. The resulting solution was diluted with water (240 ml, 19.56 rel vol) and the pH of the solution adjusted to 7.5 using 10% w/w hydrochloric acid solution (85 ml, 6.9 rel vol). The resulting suspension was stirred for 1 to 2 h at 25-30° C. The suspended solid was collected by filtration, washed with water (32 ml, 2.60 rel vol) then dried under vacuum (80-100 mbar) at 40-45° C. to provide 2-(2-chloro-5-hydroxy-4-methylcarbamoylphenoxy)-2-methylpropionic acid tert-butyl ester, weight 8.0 g (65.5%).

¹H NMR (300 MHz, CDCl₃): δ 12.44 (s, 1H), 7.33 (s, 1H), 6.37 (s, 1H), 6.15 (br s, 1H), 2.98 (d, 3H), 1.65 (s, 6H), 1.45 (s, 9H)

### Step 3. 2-[2-Chloro-4-methylcarbamoyl-5-((S)-1-oxiranylmethoxy)phenoxy]-2-methylpropionic Acid Tert-Butyl Ester

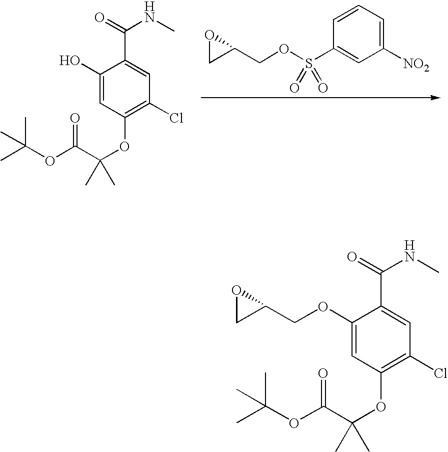

2-(2-Chloro-5-hydroxy-4-methylcarbamoylphenoxy)-2-methylpropionic acid tert-butyl ester (5.0 g, 0.0145 mol, 1.0 mol eq) was dissolved in acetonitrile (40 ml, 8.0 rel vol) and caesium carbonate (5.21 g, 5.18 g at 100%, 0.0159 mol, 1.10 mol eq) added. A solution of 3-nitrobenzenesulfonic acid (S)-1-oxiranylmethyl ester in butyronitrile (30.7% w/w, 12.89 g, 3.95 g at 100% w/w, 0.0152 mol, 1.05 mol eq) was diluted with acetonitrile (20 ml, 4.0 rel vol) and added to the reaction mixture. The reaction mixture was heated to 45-50° C. and held at this temperature for 4 h. After cooling the reaction mixture to 20 to 25° C., acetonitrile (5.0 ml, 1.0 rel vol) and water (60 ml, 12.0 rel vol) were added. The reaction mixture was stirred for 12 h at 20 to 25° C. The reaction mixture was then further cooled to 5° C. then the solid product collected by filtration and washed with water (20 ml, 4.0 rel vol). The crude product was dissolved in toluene (20 ml, 4.0 rel vol) at 40° C. then the solution was concentrated to 3.0 rel vol under vacuum (200 mbar) at around 50° C. The concentrate was cooled to 20 to 25° C. and stirred for approximately for 3 h. The solid product was collected by filtration and dried under vacuum at 40-45° C. to give 2-[2-chloro-4-methylcarbamoyl-5-((S)-1-oxiranylmethoxy)-phenoxy]-2-methylpropionic acid tert-butyl ester weight 3.8 g (65.4%).

1H NMR (300 mHz, CDCl3): δ 8.20 (s, 1H), 7.71-7.69 (broad d 1H), 6.60 (s, 1H), 4.39-4.33 (d, 1H), 4.00-3.92 (dd, 1H), 3.41-3.34 (m, 1H), 2.97-2.96 (d, 3H), 2.94-2.90 (1H, overlapping), 2.83-2.79 (m, 1H), 1.63 (s, 6H), 1.43 (s, 9H).

### Step 4. 2-{2-chloro-5-{[(2S)-3-(5-chloro-3h-spiro[1-benzofuran-2,4′-piperidin]-1′-yl)-2-hydroxypropyl]oxy}-4-[(methylamino)carbonyl]phenoxy}-2-methylpropanoic Acid Tert-Butyl Ester

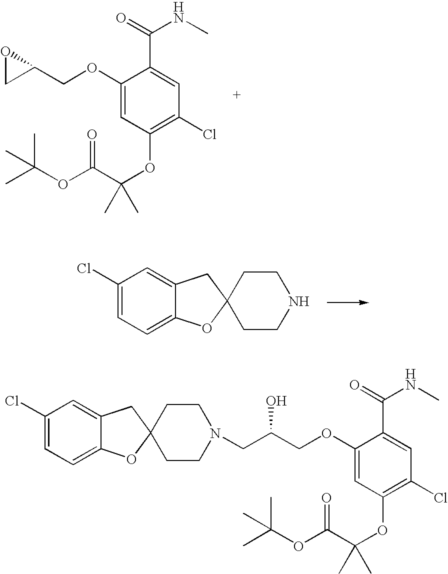

A mixture of 5-chloro-1′H,3H-spiro[1-benzofuran-2,4′-piperidine], hydrobromic acid salt (3.2 g, 0.0105 mol, 1.05 mol eq) and potassium carbonate (1.52 g, 0.011 mol, 1.10 mol eq) in ethanol (40 ml, 10.0 rel vol) was stirred for 30 min at ambient temperature. 2-[2-Chloro-4-methylcarbamoyl-5-((S)-1-oxiranylmethoxy)phenoxy]-2-methylpropionic acid tert-butyl ester (4.0 g, 0.010 mol, 1.0 mol eq) was added and the temperature of the reaction mixture raised to 48-50° C. and held for 8-9 h. The reaction mixture was cooled to 20-25° C., water (24 ml, 6.0 rel vol) was added and stirring continued for 1 h. The precipitated solid was collected by filtration and washed with water (8.0 ml, 2.0 rel vol). The solid was dissolved in ethyl acetate (30 ml, 7.5 rel vol), the resulting solution was washed with water (30 ml, 7.5 rel vol) then evaporated to dryness under vacuum (100 mbar) at 40-45° C. n-Heptane (20 ml, 5.0 rel vol) was added to the residue and the slurry stirred for 30 min. The solid was collected by filtration then dried under vacuum (150 mbar) at 40-45° C. to afford 2-{2-chloro-5-{[(2S)-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic acid tert-butyl ester, weight 4.5 g (72.1%).

¹H NMR (300 mHz, CDCl₃): δ 8.19 (s, 1H), 8.14-8.11 (broad d, 1H), 7.10-7.04 (m, 2H), 6.70-6.65 (d, 1H), 6.57 (s, 1H), 4.12-4.08 (d, 2B), 3.90-3.82 (m, 1H), 2.99-2.76 (m, 7H), 2.66-2.51 (m, 4H), 2.04-1.78 (m, 4H), 1.62 (s, 6H), 1.44 (s 9H).

### Step 5. 2-{2-Chloro-5-{[(2S)-3-(5-chloro-3H-spiro[1-benzofuran-Z 4′-piperidin]-1′-yl)-2-hydroxypropyl]oxy}-4-[(methylamino)carbonyl]phenoxy}-2-methylpropanoic Acid

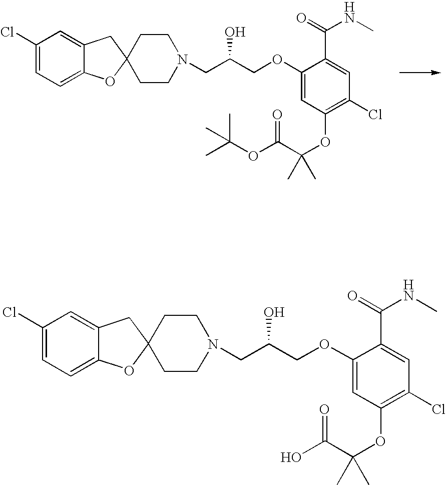

Trifluoroacetic acid (2.0 ml, 2.0 rel vol) was added to a stirred suspension of 2-{2-chloro-5-{[(2S)-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic acid tert-butyl ester (1.0 g, 0.0016 mol, 1.0 mol eq) in toluene (6.0 ml, 6.0 rel vol) at 20 to 25° C. resulting in a clear solution and stirring continued for 12 h. The reaction mixture was evaporated to dryness under reduced pressure (10 mbar) at 40° C. and the gummy residue was dissolved in water (10 ml, 10.0 rel vol). A solution of ammonium acetate (3.0 g, 0.0389 mol, 24.32 mol eq, 3.0 rel wt) in water (15 ml, 15 rel vol) was added and the thick suspension stirred for 1 to 2 h. The water layer was decanted and isopropanol (20 ml, 20.0 rel vol) added to the suspension and the mixture stirred for 30 min. The solid was collected by filtration and dried under vacuum (150 mbar) at 40° C. to provide 2-{2-chloro-5-{[(2S)-3-(5-chloro-3H-spiro[1-benzofuran-2,4′-piperidin]-1′-yl)-2-hydroxypropyl]oxy}-4-[(methylamino)carbonyl]phenoxy}-2-methylpropanoic acid, weight 0.83 g (91.2%).

APCI-MS: m/z 567 (MH⁺).

### Example 18

### {2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]-phenoxy}acetic Acid

### Step 1: 5-Chloro-4-[(4-methoxybenzyl)oxy]-N,N-dimethyl-2-[(2S)-oxiran-2-ylmethoxy]benzamide

Prepared as described in Example 5, Step 1, using dimethylamine. Purified by flash chromatography on silica gel, n-heptane/ethyl acetate mixture as mibile phase. Colourless oil. Yield 56%.

¹H-NMR (CDCl₃, 400 MHz): δ 7.36 (d, J=8.7 Hz, 2H), 7.26 (d, J=8.0 Hz, 1H), 6.90 (dd, J=11.5, 2.9 Hz, 2H), 6.59 (s, 1H), 5.08 (s, 2H), 4.26 (dd, J=11.4, 2.3 Hz, 1H), 3.84 (br.s, 1H), 3.80 (s, 3H), 3.26 (m, 1H), 2.86 (m, 4H), 2.69 (s, 1H); APCI-MS: m/z 392 (MH⁺).

### Step 2: 5-Chloro-2-{[(2S)-3-(5-chloro-1′H, 3H-spiro[1-benzofuran-2,4′-piperidin]-1′-yl)-2-hydroxypropyl]oxy}-4-hydroxy-N,N-dimethylbenzamide

Prepared from 5-chloro-4-[(4-methoxybenzyl)oxy]-N,N-dimethyl-2-[(2S)-oxiran-2-ylmethoxy]benzamide and purified as described in Example 5, Step 2. White solid, yield 65%.

¹H-NMR (acetone-d₆, 400 MHz): δ 7.23 (s, 1H), 7.18 (s, 1H), 7.13 (dd, J=8.5, 2.2 Hz, 1H), 6.91 (s, 1H), 6.77 (d, J=8.5 Hz, 1H), 4.51 (d, J=5.6 Hz, 1H), 4.12 (m, 2H), 3.90-3.72 (m, 2H), 3.60-3.43 (m, 3H), 3.38 (dd, J=13.4, 9.3 Hz, 2H), 3.17 (s, 2H), 3.04 (s, 3H), 2.94 (s, 3H), 2.43-2.18 (m, 4H); APCI-MS: m/z 495 (MH⁺).

### Step 3: Methyl {2-chloro-5-{[(2S)-3-(5-chloro-1′H, 3H-spiro[1-benzofuran-2,4′-piperidin]-1′-yl)-2-hydroxypropyl]oxy}-4-[(dimethylamino)carbonyl]phenoxy}acetate

Prepared from 5-chloro-2-{[(2S)-3-(5-chloro-1′H,3H-spiro[1-benzofuran-2,4′-piperidin]-1′-yl)-2-hydroxypropyl]oxy}-4-hydroxy-N,N-dimethylbenzamide and purified as described in Example 5, Step 3. Colourless solid, yield 71%. APCI-MS: m/z 567 (MH⁺).

### Step 4

### {2-Chloro-5-{[(2S)-3-(5-chloro-1′H, 3H-spiro[1-benzofuran-2,4′-piperidin]-1′-yl)-2-hydroxypropyl]oxy}-4-[(dimethylamino)carbonyl]phenoxy}acetic Acid

Prepared from {2-chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}acetate and purified as described in Example 5, Step 4. White solid, yield 74%.

¹H-NMR (acetone-d₆, 400 MHz): δ 7.27 (s, 1H), 7.22 (s, 1H), 7.13 (dd, J=8.5, 2.3 Hz, 1H), 6.93 (s, 1H), 6.76 (d, J=8.5 Hz, 1H), 4.91 (s, 2H), 4.50 (m, 1H), 4.18 (m, 2H), 3.86-3.43 (m, 4H), 3.35 (dd, J=13.4, 9.7 Hz, 2H), 3.27-3.11 (m, 2H), 3.05 (s, 3H), 2.93 (s, 3H), 2.42-2.18 (m, 4H); APCI-MS: m/z 553 (M).

Result from assay: IC50 (μM) 0.0114.

### Example 19

### 2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methylpropanoic Acid

### Step 1: Ethyl 2-{2-chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methylpropanoate

To a stirred solution of 5-chloro-2-{[(2S)-3-(5-chloro-1′H,3H-spiro[1-benzofuran-2,4′-piperidin]-1′-yl)-2-hydroxypropyl]oxy}-4-hydroxy-N,N-dimethylbenzamide, TFA salt, (Example 18, Step 2, 122 mg, 0.2 mmol) in DMF (3 ml) were added cesium carbonate (163 mg, 0.5 mmol) and ethyl 2-bromo-2-methylpropanoate (39 mg, 0.2 mmol). After stirring at 45° C. overnight another portion of cesium carbonate (65 mg, 0.2 mmol) and ethyl 2-bromo-2-methylpropanoate (39 mg, 0.2 mmol) were added. The reaction mixture was stirred at 50° C. for 5 h. Then the inorganic material was removed by filtration. The product was isolated by HPLC to afford the subtitle compound, as identified by APCI-MS (m/z 609 (ME)), as white solid (TFA salt, 129 mg, 89%).

### Step 2: 2-{2-Chloro-5-{[(2S)-3-(5-chloro-1′H, 3H-spiro[1-benzofuran-2,4′-piperidin]-1′-yl)-2-hydroxypropyl]oxy}-4-[(dimethylamino)carbonyl]phenoxy}-2-methylpropanoic Acid

Prepared from ethyl 2-{2-chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methylpropanoate and purified as described in Example 5, Step 4. white solid, yield 79%.

¹H-NMR (CDCl₃, 400 MHz): δ 10.32 (br.s, 1H), 7.21 (s, 1H), 7.13 (s, 1H), 7.09 (d, J=18.6 Hz, 1H), 6.68 (d, J=8.5 Hz, 1H), 6.66 (s, 1H), 4.34 (m, 1H), 4.10 (m, 2H), 3.74 (d, J=11.9 Hz, 1H), 3.56 (d, J=11.8 Hz, 1H), 3.29 (m, 3H), 3.13 (s, 3H), 3.08-2.99 (m, 3H), 2.97 (s, 3H), 2.36-2.01 (m, 4H); APCI-MS: m/z 581 (MH⁺).

Result from assay: IC50 (μM) 0.001597.

### Example 20

### (2-Chloro-5-{[(2S)-3-(5-chloro-1′H,3H-spiro[1-benzofuran-2,4′-piperidin]-1′-yl)-2-hydroxypropyl]oxy}-4-{[(3S)-3-hydroxypyrrolidin-1-yl]carbonyl]phenoxy}acetic Acid

### Step 1: Methyl (2-chloro-5-{[(2S)-3-(5-chloro-1′H,3H-spiro[1-benzofuran-2,4′-piperidin]-1′-yl)-2-hydroxypropyl]oxy)-4-([(3S)-3-hydroxypyrrolidin-1-yl]carbonyl}phenoxy)acetate

Prepared from (3S)-1-(5-chloro-2-{[(2S-3-(5-chloro-1′H,3H-spiro[1-benzofuran-2,4′-piperidin]-1′-yl)-2-hydroxypropyl]oxy}-4-hydroxybenzoyl)pyrrolidin-3-ol and purified as described in Example 5, Step 3. White solid, yield 79%. APCI-MS: m/z 609 (MHz.

### Step 2: (2-Chloro-5-{[(2S)-3-(5-chloro-1′H, 3H-spiro[1-benzofuran-2,4′-piperidin-]-1′-yl)-2-hydroxypropyl]oxy}-4-{[(3S)-3-hydroxypyrrolidin-1-yl]carbonyl}phenoxy)acetic Acid

Prepared from methyl (2-chloro-5-{[(2S)-3-(5-chloro-1′H,3H-spiro[1-benzofuran-2,4′-piperidin]-1′-yl)-2-hydroxypropyl]oxy}-4-{[(3S)-3-hydroxypyrrolidin-1-yl]carbonyl}phenoxy)acetate and purified as described in Example 5, Step 4. White solid, yield 77%.

¹H-NMR (acetone-d₆, 400 MHz): δ 7.32 (d, J=1.8 Hz, 1H), 7.22 (s, 1H), 7.13 (dd, J=8.5, 1.5 Hz, 1H), 6.93 (s, 1H), 6.77 (d, J=8.5 Hz, 1H), 4.93 (d, J=1.2 Hz, 2H), 4.51 (br.s, 2H), 4.42 (m, 1H), 4.19-3.34 (m, 7H), 3.30 (t, J=10.9 Hz, 2H), 3.17 (s, 2H), 2.43-2.17 (m, 4H), 2.05-1.88 (m, 2H, partially covered with the signal of solvent); APCI-MS: m/z 595 (MH⁺).

Result from assay: IC50 (μM) 0.00202.

### Example 21

### 2-{2-Chloro-5-{3-(5-chloro-1′H,3H-spiro[1-benzofuran-2,4′-piperidin]-1′-yl)-2-hydroxypropyl]oxy}-4-[(dimethylamino)carbonyl]phenyl}-2-methyl-propanoic Acid Trifluoracetate

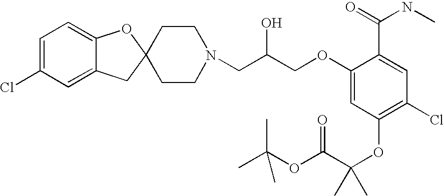

### Step 1: Tert-Butyl 2-{2-chloro-5-{[3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoate

To a stirred solution of racemic 5-chloro-2-{[3-(5-chloro-1′H,3H-spiro[1-benzofuran-2,4′-piperidin]-1′-yl)-2-hydroxypropyl]oxy}-4-hydroxy-N-methylbenzamide (230 mg, 0.48 mmol) and tert-butyl-2-bromoisobutyrate (266 μL, 1.43 mmol) in dry DMF (2 ml) was added cesiumcarbonate (466 mg, 1.43 mmol) and the mixture stirred at 60° C. overnight. The mixture was partitioned between ethylacetate and water. The organic phase was washed with water, dried and evaporated to give the titled compound as an orange oil (300 mg)

APCI-MS: m/z 623/625

### Step 2: 2-{2-Chloro-5-{3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoic Acid Trifluoracetate (Salt)

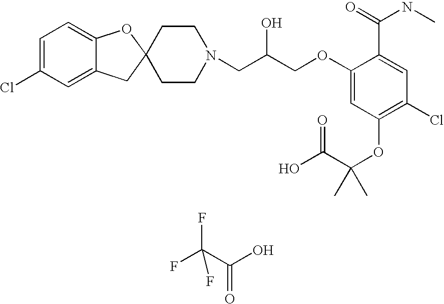

A solution of racemic tert-butyl 2-{2-chloro-5-{[3-(5-chloro-1′H,3H-spiro[1-benzofuran-2,4′-piperidin]-1′-yl)-2-hydroxypropyl]oxy}-4-[(dimethylamino)-carbonyl]phenoxy}-2-methyl-propanoate (300 mg, 0.48 mmol), triethylsilane (scavenger, 200 μL, 1.25 mmol) and TFA (0.5 ml) in DCM (2 ml) was stirred at ambient temperature 1 h. After evaporation the residue was purified by RP prep-HPLC using acetonitrile and water containing 0.1% TFA as mobil phase in gradient. Pooled fractions were freezedried. The titled compound was obtained as a white solid (152 mg, 46%)

¹H NMR (299.946 MHz, dmso) δ 9.86 (bs, 1H), 8.13 (q, J=9.1 Hz, 1H), 7.75 (s, 1H), 7.29 (s, 1H), 7.16 (dd, J=8.5, 2.3 Hz, 1H), 6.80 (d, J=8.5 Hz, 1H), 6.62 (s, 1H), 4.49-4.34 (m, 1H), 4.01 (d, J=4.5 Hz, 2H), 3.67-3.04 (m, 9H), 2.82 (d, J=4.6 Hz, 3H), 2.27-2.00 (m, 4H), 1.60 (s, 6H)

APCI-MS m/z 567/569 (MH⁺)

Result from assay: IC50 (μM) 0.002424.

### Example 22

### 2-[5-{[(2S)-3-(7-tert-Butyl-5-chloro-1′H,3H-spiro[1-benzofuran-2,4′-piperidin]-1′-yl)-2-hydroxypropyl]oxy}-2-chloro-4-(methylcarbamoyl)phenoxy]-2-methylpropanoic Acid

### Step 1: 2-{[(2S)-3-(7-tert-Butyl-5-chloro-1′H),3H-spiro[1-benzofuran-2,4′-piperidin]-1′-yl)-2-hydroxypropyl]oxy}-4-hydroxy-N-methylbenzamide Trifluoroacetate

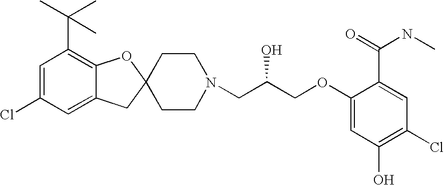

2-{[(2S)-3-(7-tert-Butyl-5-chloro-1′H,3H-spiro[1-benzofuran-2,4′-piperidin]-1′-yl)-2-hydroxypropyl]oxy}-4-methoxybenzyl)oxy]-N-methylbenzamide (300 mg, 0.46 mmol, isolated as an impurity from crude 5-chloro-2-{[(2S)-3-(5-chloro-1′H,3H-spiro[1-benzofuran-2,4′-piperidin]-1′-yl)-2-hydroxypropyl]oxy}-4-methoxy-benzyl)oxy]-N-methylbenzamide) was dissolved in DCM (3 ml) and TFA (3 ml) was added. After standing 1.5 h the reaction was complete and the reaction mixture worked up by evaporation and purification by prep-HPLC using acetonitrile and water containing 0.1% TFA as mobil phase yielding the titled compound as an oil, (75 mg, 25%).

¹H NMR (299.946 MHz, acetone) δ 8.11-8.01 (m, 1H), 7.88 (s, 1H), 7.09 (d, J=2.1 Hz, 1H), 7.04 (d, J=2.3 Hz, 1H), 6.88 (s, 1H), 4.81-4.67 (m, 1H), 4.30-4.19 (m, 2H), 4.09-3.92 (m, 2H), 3.77-3.49 (m, 4H), 3.16 (s, 2H), 2.91 (d, J=14.0 Hz, 3H), 2.56-2.17 (m, 4H), 1.39 (s, 9H)

APCI-MS m/z 537/539 (MH+)

### Step 2: 2-[5-{[(2S)-3-(7-tert-Butyl-5-chloro-1′H,3H-spiro[1-benzofuran-2,4′-piperidin]-1′-yl)-2-hydroxypropyl]oxy}-2-chloro-4-(methylcarbamoyl)phenoxy-2-methylpropanoic Acid Trifluoroacetate

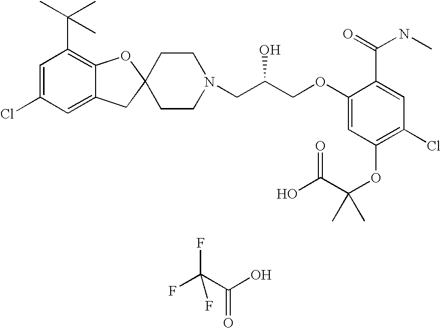

To 2-{[(2S)-3-(7-tert-butyl-5-chloro-1′H,3H-spiro[1-benzofuran-2,4′-piperidin]-1′-yl)-2-hydroxypropyl]oxy}-4-hydroxy-N-methylbenzamide trifluoroacetate (75 mg, 115 μmol) and ethyl 2-bromoisobutyrate (85 μL, 575 μmol) dissolved in dry DMF (2 ml) was added cesiumcarbonate (188 mg, 575 μmol) and the mixture stirred at 60° C. 3 h. The crude product obtained after extractive work-up from ethylacetate and water was hydrolyzed in 1,4-dioxane (1 ml) and water (1 ml) containing 1 M sodium-hydroxide (100 μL) at 70° C. 2 h. The reaction mixture was acidified with TFA and evaporated to give an oil (76 mg). Pooled crude material from two batches (126 mg) was purified by prep-HPLC using acetonitrile and water containing 0.1% TFA as mobil phase and the appropriate fractions freezedried to yield the titled compound as a white amorphous solid (100 mg).

¹H NMR (299.946 MHz, acetone) δ 8.02-7.94 (m, 1H), 7.97 (s, 1H), 7.09 (d, J=2.1 Hz, 1H), 7.04 (d, J=2.3 Hz, 1H), 6.78 (s, 1H), 4.75-4.65 (m, 1H), 4.27-4.16 (m, 2H), 4.01-3.79 (m, 2H), 3.74-3.46 (m, 4H), 3.17 (s, 2H), 2.90 (d, J=4.7 Hz, 3H), 2.50-2.22 (m, 4H), 1.66 (s, 6H), 1.36 (s, 9H)

APCI-MS m/z 623/625 (MH+)

Result from assay: IC50 (μM) 0.0717.

### Example 23

### Human CCR1 Binding Assay

### Membranes

HEK293 cells, from ECACC, stably expressing recombinant human CCR1 (HEK-CCR1) were used to prepare cell membranes containing CCR1. The membranes were stored at −70° C. The concentration of membranes of each batch was adjusted to 10% specific binding of 33 pM [¹²⁵I] MIP-1α.

### Binding Assay

100 μL of HEK-CCR1 membranes diluted in assay buffer pH 7.4 ((137 mM NaCl (Merck, Cat No 1.06404), 5.7 mM Glucose (Sigma, Cat No G5400), 2.7 mM KCl (Sigma, Cat No P-9333), 0.36 mM NaH₂PO₄×H₂O (Merck, Cat No 1.06346), 10 mM HEPES (Sigma, Cat No H3375), 0.1% (w/v) Gelatine (Sigma, Cat No G2625)) with the addition of 17500 units/L Bacitracin (Sigma, Cat No B 1025) were added to each well of the 96 well filter plate (0.45 μm opaque Millipore cat no MHVB N4550). 12 μL of compound in assay buffer, containing 10% DMSO, was added to give final compound concentrations of 1×10^{-5.5}-1×10^{-9.5} M. 12 μL cold human recombinant MIP-1α (270-LD-050, R&D Systems, Oxford, UK), 10 nM final concentration in assay buffer supplemented with 10% DMSO, was included in certain wells (without compound) as non-specific binding control (NSB). 12 μL assay buffer with 10% DMSO was added to certain wells (without compound) to detect maximal binding (B0).

12 μL [¹²⁵I] MIP-1α, diluted in assay buffer to a final concentration in the wells of 33 pM, was added to all wells. The plates with lid were then incubated for 1.5 h at room temperature. After incubation the wells were emptied by vacuum filtration (MultiScreen Resist Vacuum Manifold system, Millipore) and washed once with 200 μl assay buffer. After the wash, all wells received an addition of 50 μL of scintillation fluid (OptiPhase “Supermix”, Wallac Oy, Turko, Finland). Bound [¹²⁵I] MIP-1α was measured using a Wallac Trilux 1450 MicroBeta counter. Window settings: Low 5-High 1020, 1-minute counting/well.

### Calculation of Percent Displacement and IC₅₀

The following equation was used to calculate percent displacement.

Percent displacement=1−((cpm test−cpm NSB)/(cpm B0−cpm NSB)) where:

cpm test=average cpm in wells with membranes and compound and [¹²⁵I] MIP-1α;
NSB=average cpm in the wells with membranes and MIP-1α and [¹²⁵I] MIP-1α (non-specific binding);
B0=average cpm in wells with membranes and assay buffer and [¹²⁵I] MIP-1α (maximum binding).

The molar concentration of compound producing 50% displacement (IC₅₀) was derived using the Excel-based program XLfit (version 2.0.9) to fit data to a 4-parameter logistics function.

### Example 24

### Human CCR3 Binding Assay

### Membranes

CHO-K1 cells, from ATCC, stably expressing recombinant human CCR3 (CHO—CCR3) were used to prepare cell membranes containing CCR3. The membranes were stored at −70° C. A membrane concentration was used which gave approximately 10% specific binding relative to the total amount of radioactivity of [³H]-4-(2,4-dichloro-3-methylphenoxy)-1′-[4-(methylsulfonyl)benzoyl]-1,4′-bipiperidine added to the assay.

### Binding Assay

[³H]-4-(2,4-dichloro-3-methylphenoxy)-1′-[4-(methylsulfonyl)benzoyl]-1,4′-bipiperidine (20 μL, to a final concentration of 2 nM, pre-diluted in assay buffer from a 20 μM stock) and either vehicle (20 μL, 10% (v/v) DMSO in assay buffer: for determination of total binding (B0)), 1,4′-bipiperidine, 4-(2,4-dichloro-3-methylphenoxy)-1′-[4-(methylsulfonyl)benzoyl] (20 μL, 100 μM solution in 10% (v/v) DMSO in assay buffer: for determination of non-specific binding (NSB)) or the appropriate solution of test compound (20 μL, 10% (v/v) DMSO in assay buffer) were added to the wells of a U-bottomed 96-well plate. Membranes pre-diluted in assay buffer (160 μL) were then added, giving a total incubation volume of 200 μL per well.

The plates were sealed and incubated for 2 h at room temperature. The plates were then filtered onto GF/B filter plates, pre-soaked for 1 h in plate-coating solution, using a 96-well plate Tomtec cell harvester. Four washes with wash buffer (200 μL) were performed at 4° C. to remove unbound radioactivity. The plates were dried either for at least 2 h at 50° C. or over night at room temperature. Filtration plates were sealed from underneath using Packard plate sealers (supplied with plates) and of MicroScint-O (50 μL) was added to each well. The plates were sealed (TopSeal A) and filter-bound radioactivity was measured with a scintillation counter (TopCount, Packard BioScience) using a 1 minute counting protocol.

### Calculation of Percent Displacement and IC₅₀

The molar concentration of test compound producing 50% displacement (IC₅₀) of [³H]-4-(2,4-dichloro-3-methylphenoxy)-1′-[4-(methylsulfonyl)benzoyl]-1,4′-bipiperidine specific binding (B0-NSB) was derived utilising GraphPad Prism® to fit data to a 4-parameter logistic function of the form:

$$E = β + \frac{{α [ B ]}^{m}}{{[ B ]}^{m} + {IC}_{50}^{m}}$$

in which E and [B] are specific binding of [³H]-4-(2,4-dichloro-3-methylphenoxy)-1′-[4-(methylsulfonyl)benzoyl]-1,4′-bipiperidine, and concentration of the antagonist respectively; α, β, IC₅₀ and m are the asymptote, baseline, location and slope parameters, respectively. The derived IC₅₀ values were transformed to the negative logarithm (pIC₅₀) and then corrected using the Cheng-Prusoff equation to give pKi values for calculation of descriptive statistics (mean±SEM).

### Example 25

### hERG-Encoded Potassium Channel Binding Assay

This assay, which is described in full detail in example 2, WO2005037052, determines the ability of a test compound to bind to the human ether-a-go-go-related-gene (hERG)-encoded potassium channel. The assay comprises the following steps: a) incubation of HEK 293 cell membranes expressing the I_{KR} channel in the presence of radioligand 3,7-bis[2-(4-nitro[3,5-^{'}H]phenyl)ethyl]-3,7-diazabicyclo[3.3.1]nonane, in the presence or absence of a test compound; b) quantitation of specifically bound labelled compound in the presence or absence of a test compound; c) calculation of the inhibition of labelled compound binding by the test compound. Similar protocols to determine affinity for the human ether-a-go-go-related-gene (hERG)-encoded potassium channel have been described by Finlayson, K. et al. [Eur. J. Pharmacol. 2001, 412, 203 and Eur. J. Pharmacol. 2001, 430, 147].

### Example 26

### hERG-Encoded Potassium Channel Inhibition Assay

This assay determines the ability of a test compound to inhibit the tail current flowing through the human ether-a-go-go-related-gene (hERG)-encoded potassium channel.

Human embryonic kidney (HEK) cells expressing the hERG-encoded channel were grown in Minimum Essential Medium Eagle (EMEM; Sigma-Aldrich catalogue number M2279), supplemented with 10% Foetal Calf Serum (Labtech International; product number 4-101-500), 10% M1 serum-free supplement (Egg Technologies; product number 70916) and 0.4 mg/ml Geneticin G418 (Sigma-Aldrich; catalogue number G7034). One or two days before each experiment, the cells were detached from the tissue culture flasks with Accutase (TCS Biologicals) using standard tissue culture methods. They were then put onto glass coverslips resting in wells of a 12 well plate and covered with 2 ml of the growing media.

For each cell recorded, a glass coverslip containing the cells was placed at the bottom of a Perspex chamber containing bath solution (see below) at room temperature (˜20° C.). This chamber was fixed to the stage of an inverted, phase-contrast microscope. Immediately after placing the coverslip in the chamber, bath solution was perfused into the chamber from a gravity-fed reservoir for 2 min at a rate of ˜2 ml/min. After this time, perfusion was stopped.

A patch pipette made from borosilicate glass tubing (GC120F, Harvard Apparatus) using a P-97 micropipette puller (Sutter Instrument Co.) was filled with pipette solution (see hereinafter). The pipette was connected to the headstage of the patch clamp amplifier (Axopatch 200B, Axon Instruments) via a silver/silver chloride wire. The headstage ground was connected to the earth electrode. This consisted of a silver/silver chloride wire embedded in 3% agar made up with 0.85% sodium chloride.

The cell was recorded in the whole cell configuration of the patch clamp technique. Following “break-in”, which was done at a holding potential of −80 mV (set by the amplifier), and appropriate adjustment of series resistance and capacitance controls, electrophysiology software (Clampex, Axon Instruments) was used to set a holding potential (−80 mV) and to deliver a voltage protocol. This protocol was applied every 15 seconds and consisted of a 1 s step to +40 mV followed by a 1 s step to −50 mV. The current response to each imposed voltage protocol was low pass filtered by the amplifier at 1 kHz. The filtered signal was then acquired, on line, by digitising this analogue signal from the amplifier with an analogue to digital converter. The digitised signal was then captured on a computer running Clampex software (Axon Instruments). During the holding potential and the step to +40 mV the current was sampled at 1 kHz. The sampling rate was then set to 5 kHz for the remainder of the voltage protocol.

The compositions, pH and osmolarity of the bath and pipette solution are tabulated below.

| Salt | Pipette (mM) | Bath (mM) |
|---|---|---|
| NaCl | — | 137 |
| KCl | 130 | 4 |
| MgCl₂ | 1 | 1 |
| CaCl₂ | — | 1.8 |
| HEPES | 10 | 10 |
| glucose | — | 10 |
| Na₂ATP | 5 | — |
| EGTA | 5 | — |

| Parameter | Pipette | Bath |
|---|---|---|
| pH | 7.18-7.22 | 7.40 |
| pH adjustment with | 1 M KOH | 1 M NaOH |
| Osmolarity (mOsm) | 275-285 | 285-295 |

The amplitude of the hERG-encoded potassium channel tail current following the step from +40 mV to −50 mV was recorded on-line by Clampex software (Axon Instruments). Following stabilisation of the tail current amplitude, bath solution containing the vehicle for the test substance was applied to the cell. Providing the vehicle application had no significant effect on tail current amplitude, a cumulative concentration effect curve to the compound was then constructed.

The effect of each concentration of test compound was quantified by expressing the tail current amplitude in the presence of a given concentration of test compound as a percentage of that in the presence of vehicle.

Test compound potency (IC₅₀) was determined by fitting the percentage inhibition values making up the concentration-effect to a four parameter Hill equation using a standard data-fitting package. If the level of inhibition seen at the highest test concentration did not exceed 50%, no potency value was produced and a percentage inhibition value at that concentration was quoted.

Compounds of the invention showed affinity in the hCCR1 assay (Example 23) at concentrations of less than 20 nM. In the hERG assays (Examples 25 and 26) however, the IC₅₀ value was in excess of 20 μM. For example, the compound of Example 1 showed a hERG binding concentration of >30 μM, which is at least an order of magnitude greater than related compounds of the prior art; such as the compound described in Example 83 of WO2004005295.

1-67. (canceled)

68. A compound of formula 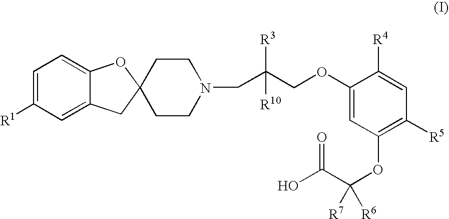
wherein:
R¹ is halogen;
R³ is hydrogen or hydroxyl;
R¹⁰ is hydrogen or C₁₋₃alkyl;
R⁴ is —CONR⁸R⁹, —N(H)C(O)R¹¹ or —N(H)C(O)NR⁸R⁹, where R⁸ and R⁹ are independently selected from hydrogen, C₁₋₆ alkyl or C₃₋₇cycloalkyl, or R⁸ and R⁹ together with the nitrogen atom to which they are attached, form a 4-7 membered heterocyclic ring which is optionally substituted with one or more hydroxy groups;
R¹¹ is C₁₋₆alkyl, C₂₋₆alkenyl, C₃₋₆cycloalkyl, adamantyl, C₅₋₆cycloalkenyl, phenyl or a saturated or unsaturated 5-10 membered heterocyclic ring system comprising at least one heteroatom selected from nitrogen, oxygen, and sulphur, each of which may be optionally substituted by one or more substituents independently selected from nitro, hydroxyl, oxo, halo, carboxyl, C₁₋₆ alkyl, C₁₋₆alkoxy, C₁₋₆alkylthio, C₁₋₆alkycarbonyl, C₁₋₆alkoxycarbonyl, phenyl or —NHC(O)R²;
R² is C₁₋₆alkyl, amino or phenyl;
R⁵ is hydrogen or halo;
R⁶ and R⁷ are independently selected from hydrogen or C₁₋₆alkyl, or R⁶ and R⁷ together with the carbon atom to which they are attached form a 3-7 membered saturated cycloalkyl group, or a pharmaceutically acceptable salt thereof.

69. A compound according to claim 68 wherein R¹ is selected from chlorine and fluorine.

70. A compound according to any one of claims 68 or 69 wherein R³ is hydroxyl.

71. A compound according to any one of the preceding claims wherein R¹⁰ is hydrogen.

72. A compound according to any one of the preceding claims wherein R⁴ is —CONR⁸R⁹ or —N(H)C(O)NR⁸R⁹, where R⁸ and R⁹ are selected from hydrogen or C₁₋₆ alkyl.

73. A compound according to any one of the preceding claims wherein R⁵ is hydrogen or chlorine.

74. A compound according to any one of the preceding claims wherein R⁶ and R⁷ are independently selected from hydrogen or C₁₋₆ alkyl.

75. A compound of formula (IA) 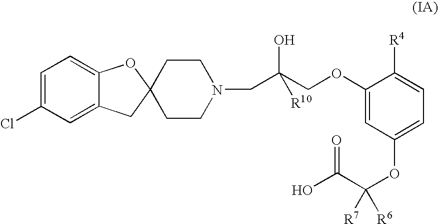
where R⁴, R⁶, R⁷ and R¹⁰ are as defined in claim 68, or a pharmaceutically acceptable salt thereof.

76. A compound of formula (IB) 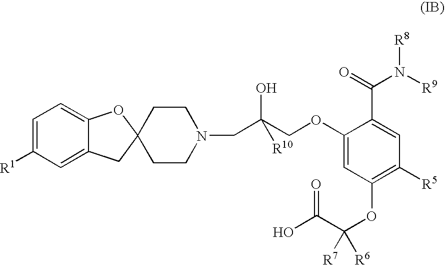
where R¹, R⁵, R⁶, R⁷, R⁸, R⁹ and R¹⁰ are as defined in claim 68, or a pharmaceutically acceptable salt thereof.

77. A compound of formula (IC) 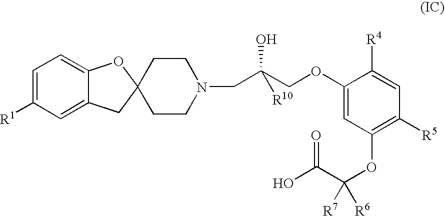
where R¹, R⁴, R⁵, R⁶, R⁷ and R¹⁰ are as defined in claim 68 or a pharmaceutically acceptable salt thereof.

78. A compound according to any one of the preceding claims which is in zwitterionic forms.

79. A compound selected from:
(4-(acetylamino)-2-chloro-5-{[(2S)-3-(5-chloro-1′H,3H-spiro[1-benzofuran-2,4′-piperidin]-1′-yl)-2-hydroxypropyl]oxy}phenoxy)acetic acid;
(4-(acetylamino)-3-{[(2S)-3-(5-chloro-1′H,3H-spiro[1-benzofuran-2,4′-piperidin]-1′-yl)-2-hydroxypropyl]oxy}phenoxy)acetic acid;
(4-(acetylamino)-2-chloro-5-{[(2S)-3-(5-fluoro-1′H,3H-spiro[1-benzofuran-2,4′-piperidin]-1′-yl)-2-hydroxypropyl]oxy}phenoxy)acetic acid;
{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}acetic acid;
2-{2-Chloro-5-{[(2S)-3-(5-chloro-1′H,3H-spiro[1-benzofuran-2,4′-piperidin]-11′-yl)-2-hydroxypropyl]oxy}-4-[(methylamino)carbonyl]phenoxy}-2-methylpropanoic acid;
{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}acetic acid;
2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methylpropanoic acid;
(2-Chloro-5-{[(2S)-3-(5-chloro-1′H,3H-spiro[1-benzofuran-2,4′-piperidin]-1′-yl)-2-hydroxypropyl]oxy}-4-{[(3S)-3-hydroxypyrrolidin-1-yl]carbonyl]phenoxy}acetic acid;
2-{2-Chloro-5-{[(2R)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid;
2-{2-Chloro-5-{3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoic acid; and
or a pharmaceutically acceptable salt thereof.

80. A compound selected from:
(4-(acetylamino)-2-chloro-5-{[(2S)-3-(5-fluoro-1′H,3H-spiro[1-benzofuran-2,4′-piperidin]-1′-yl)-2-hydroxypropyl]oxy}phenoxy)acetic acid, hydrochloride;
2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid sodium hydroxide;
2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methylpropanoic acid hydrochloride;
2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoic acid trifluoracetate;
2-{2-Chloro-5-{[(2S)-3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoic acid p-toluensulfonat;
2-{2-Chloro-5-{3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoic acid trifluoracetate; and
2-[5-{[(2S)-3-(7-tert-Butyl-5-chloro-1′H,3H-spiro[1-benzofuran-2,4′-piperidin]-1′-yl)-2-hydroxypropyl]oxy}-2-chloro-4-(methylcarbamoyl)phenoxy]-2-methylpropanoic acid trifluoroacetate.

81. The compound 2-[5-{[(2S)-3-(7-tert-Butyl-5-chloro-1′H,3H-spiro[1-benzofuran-2,4′-piperidin]-1′-yl)-2-hydroxypropyl]oxy}-2-chloro-4-(methylcarbamoyl)phenoxy]-2-methylpropanoic acid, or a pharmaceutically acceptable salt thereof, and 2-[5-{[(2S)-3-(7-tert-Butyl-5-chloro-1′H,3H-spiro[1-benzofuran-2,4′-piperidin]-1′-yl)-2-hydroxypropyl]oxy}-2-chloro-4-(methylcarbamoyl)phenoxy]-2-methylpropanoic acid trifluoroacetate.

82. The compound 2-{2-Chloro-5-{3-(5-chloro-1′H,3H-spiro[1-benzofuran-2,4′-piperidin]-1′-yl)-2-hydroxypropyl]oxy}-4-[(dimethylamino)carbonyl]phenoxy}-2-methyl-propanoic acid.

83. The compound 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid.

84. A pharmaceutical composition comprising a compound of formula I or a pharmaceutically acceptable salt thereof, as claimed in any one of claims 68 to 83, in association with a pharmaceutically acceptable adjuvants, diluents and/or carriers.

85. A pharmaceutical composition according to claim 84, which further comprises an additional therapeutic agent.

86. A pharmaceutical device comprising a compound according to any one of claims 68 to 83 or a composition according to claim 84 or 85.

87. A compound of formula I or a pharmaceutically acceptable salt thereof, as claimed in any one of claims 68 to 83 for use in therapy.

88. Use of a compound of formula I or a pharmaceutically acceptable salt thereof, as claimed in any one of claims 68 to 83, in the manufacture of a medicament for treating a respiratory disease.

89. Use of a compound of formula I or a pharmaceutically acceptable salt thereof, as claimed in any one of claims 68 to 83, in the manufacture of a medicament for treating airway diseases, inflammatory diseases, COPD and/or asthma.

90. A method of treatment of respiratory diseases, airway diseases, inflammatory diseases, COPD and/or asthma, in a patient suffering from, or at risk of, said disease, which comprises administering to the patient a therapeutically effective amount of the compound of formula I or a pharmaceutically acceptable salt thereof, as claimed in any one of claims 68 to 83.

91. The method according to claim 90 whereby the compound of formula I or a pharmaceutically acceptable salt thereof, as claimed in any one of claims 68 to 83 is administered by inhalation.

92. A process for the preparation of a compound of formula (I) or a pharmaceutically acceptable salt thereof as defined in claim 68 which comprises;
(a) where R³ is a hydroxyl group, reacting a compound of formula (II) 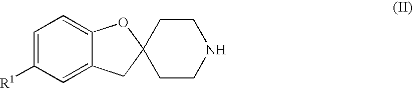
where R¹ is as defined in claim 68, with a compound of formula (III) 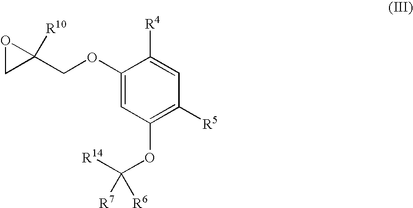
where R⁴, R⁵, R⁶, R⁷ and R¹⁰ are as defined in claim 68, or a protected derivative thereof, and R¹⁴ is carboxy or a protected derivative thereof, or
(b) where R³ is a hydroxyl group, reacting a compound of formula (IV) 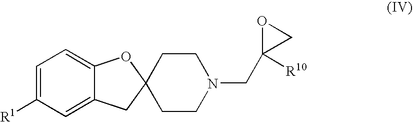
where R¹ and R¹⁰ are as defined in claim 68, with a compound of formula (V) 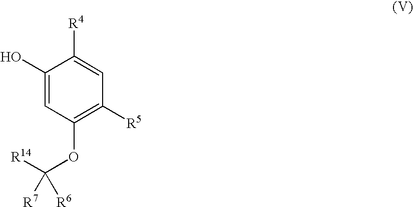
where R⁴, R⁵, R⁶ and R⁷ are as defined in claim 68, in the presence of a suitable base, and R¹⁴ is carboxy or a protected derivative thereof: or
(c) reacting a compound of formula (II) as defined above, with a compound of formula (VI) 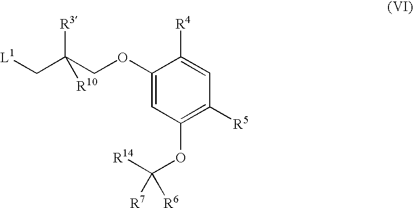
wherein L¹ is a leaving group R⁴, R⁵, R⁶, R⁷ and R¹⁰ are as defined claim 68, and R¹⁴ is carboxy or a protected derivative thereof, R^{3′} is R³ as defined in claim 68 or —O—P where P is a suitable protecting group,
(d) reacting a compound of formula (VII) 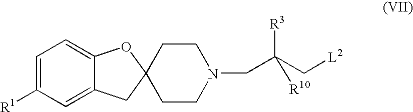
where R¹, R³ and R¹⁰ are as defined in claim 68, L² is a suitable leaving group, with a compound of formula (V) as defined above; in the presence of a base,
(e) when R⁴ represents a group —N(H)C(O)R¹¹, reacting a compound of formula (IX) 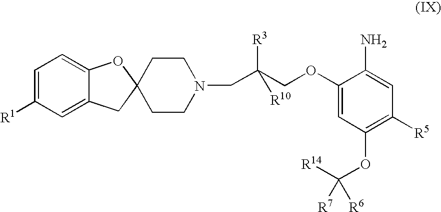
where R¹, R³, R⁵, R⁶, R⁷ and R¹⁰ are as defined in claim 68 and R¹⁴ is carboxy or a protected derivative thereof,
with a compound of formula (X) 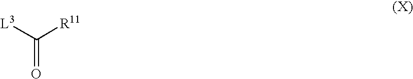
where R¹¹ is as defined in claim 68, and L³ is a leaving group;
(f) when R⁴ represents a group —CONR⁸R⁹, reacting a compound of formula (XI) 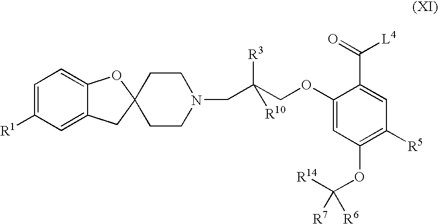
where R¹, R³, R⁵, R⁶, R⁷ and R¹⁰ are as defined in claim 68, R¹⁴ is carboxy or a protected derivative thereof and L⁴ is a leaving group with a compound of formula (XII)
HNR⁸R⁹ (XII)
where R⁸ and R⁹ are as defined in claim 68;
(g) reacting a compound of formula (XIII) 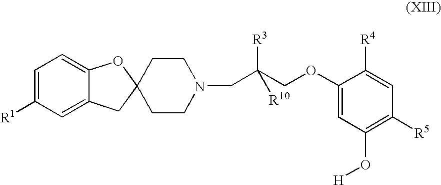
where R¹, R³, R⁴, R⁵ and R¹⁰ are as defined in claim 68, with a compound of formula (XIV) 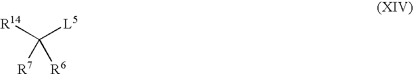
where R⁶ and R⁷ are as defined in claim 68, L⁵ is a leaving group and R¹⁴ is carboxy or a protected derivative thereof in the presence of a base;
and thereafter, if desired or necessary, carrying out one or more of the following steps
(i) converting a compound of formula (I) obtained to a different compound of formula (I);
(ii) removing any protecting groups; and
(iii) forming a pharmaceutically acceptable salt of the compound of formula (I).

93. A compound of formula (III) 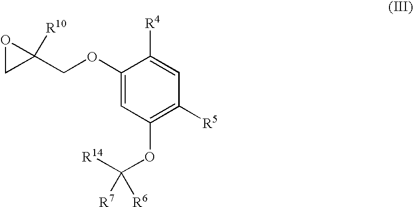
wherein R⁴, R⁵, R⁶, R⁷ and R¹⁰ are as defined in claim 68, or a protected derivative thereof, and R¹⁴ is carboxy or a protected derivative thereof, or a salt thereof.

94. A compound of formula (V) 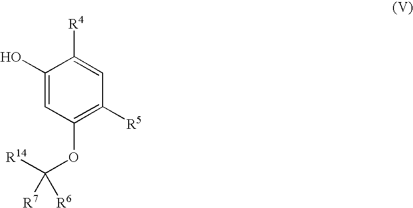
where R⁴, R⁵, R⁶ and R⁷ are as defined in claim 68, in the presence of a suitable base, and R¹⁴ is carboxy or a protected derivative thereof.

95. A compound of formula (VI) 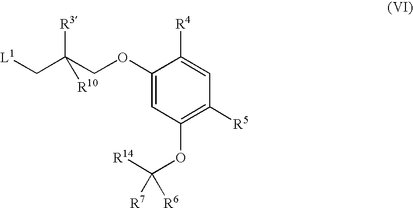
wherein L¹ is a leaving group, R⁴, R⁵, R⁶, R⁷ and R¹⁰ are as defined in claim 68, R¹⁴ is carboxy or a protected derivative thereof, R^{3′} is R³ as defined in claim 68 or —O—P where P is a protecting group, or a salt thereof.

96. A compound of formula (IX) 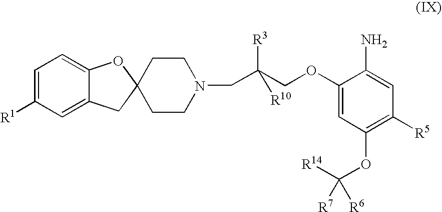
where R¹, R³, R⁵, R⁶, R⁷ and R¹⁰ are as defined in claim 68 and R¹⁴ is carboxy or a protected derivative thereof, or a salt thereof.

97. A compound of formula (XI) 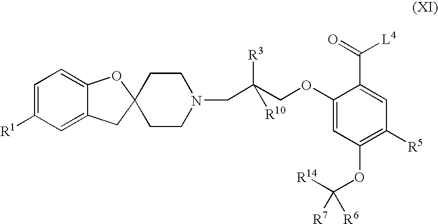
where R¹, R³, R⁵, R⁶, R⁷ and R¹⁰ are as defined in claim 68, R¹⁴ is carboxy or a protected derivative thereof and L⁴ is a leaving group, or a salt thereof.
